(19) United States
(12) Patent Application Publication
(10) Pub. No.: US 2019/0038671 A1
(43) Pub. Date: Feb. 7, 2019

(54) ENGINEERED MAMMALIAN CELLS FOR CANCER THERAPY

(71) Applicant: Nanjing Legend Biotech Co., Ltd., Nanjing, Jiangsu (CN)

(72) Inventors: Xiaohu FAN, Edmonton, Alberta (CA); Fangliang ZHANG, Nanjing, Jiangsu (CN); Qiuchuan ZHUANG, Nanjing, Jiangsu (CN); Pingyan WANG, Fengyang, Anhui (CN); Jie YU, Nanjing, Jiangsu (CN); Xian HE, Nanjing, Jiangsu (CN); Lin WANG, Nanjing, Jiangsu (CN); Jiaying HAO, Nanjing, Jiangsu (CN); Lei YANG, Huainan, Anhui (CN)

(21) Appl. No.: 16/075,220

(22) PCT Filed: Jan. 26, 2017
(86) PCT No.: PCT/CN2017/072723

§ 371 Date: Aug. 3, 2018

(30) Foreign Application Priority Data
Feb. 4, 2016 (CN) PCT/CN2016/073489
Jun. 30, 2016 (CN) PCT/CN2016/087855

(51) Int. Cl. A61K 35/17; A61K 39/00; A61P 35/00; C12N 5/0783; C07K 16/28; C07K 16/32

(52) U.S. Cl. CPC A61K 35/17; A61K 39/0011; A61P 35/00; C12N 5/0636; C07K 16/2809; A61K 2039/572; C07K 16/32; A61K 2039/5158; A61K 2039/507; A61K 2039/5156; C07K 16/2863

(57) ABSTRACT

The present invention provides a cell-based platform for controllable, regionalized, and cost-effective delivery of immunomodulator and other therapeutic proteins, which is widely applicable in cancer immunotherapy.

### CROSS REFERENCE TO RELATED APPLICATIONS

This application claims priority benefit of International Patent Application No. PCT/CN 2016/073489 filed Feb. 4, 2016, and International Patent Application No. PCT/CN 2016/087855 filed Jun. 30, 2016, the contents of each of which are incorporated herein by reference in their entirety.

### FIELD OF THE INVENTION

The present invention relates to pharmaceutical compositions comprising engineered mammalian cells that express therapeutic proteins and methods of use thereof for cancer immunotherapy.

### BACKGROUND OF THE INVENTION

The immune surveillance hypothesis proposes that the immune system plays an important role in inhibiting tumor growth. The immune system can distinguish tumor cells from normal cells by recognizing tumor associated antigens. T cells are one of the major types of immune cells that play key roles in cancer immunity. In the theory of immunoediting, a fraction of tumor cells “escape” from the surveillance and the clearance of the immune system, become less immunogenic, and eventually grow into clinically significant tumors. The “escape” may include several activities by the tumor cells, such as down-regulation of co-stimulatory molecule expression, and up-regulation of inhibitory molecule expression. The response of the T cells to the tumor cells is regulated by the balance between the inhibitory signals and the co-stimulatory signals.

Several cancer immunotherapy strategies have been recently explored. Blockade of inhibitory immune checkpoints, such as PD-1 and CTLA-4, has been increasingly considered as an attractive strategy for cancer immunotherapy. Blake et al reported that blockade of PD-1/PD-L1 with an anti-PD-L1 antibody promotes adoptive T-cell immunotherapy in a tolerogenic environment (2015). An alternative strategy is cancer vaccines, which introduce heterologous genes to human bodies for clinical purposes. In October 2015, the U.S. FDA approved the injectable formulation of T-VEC (IMLYGIC®) for the treatment of melanoma in patients with inoperable tumors. Engineered from Herpes Simplex Virus 1 (HSV-1), T-VEC is an oncolytic virus encoding a GM-CSF cytokine gene, which preferentially replicates in cancer cells. T-VEC infected cancer cells secret GM-CSF, which attract DC cells, and thereafter facilitate the cytotoxic T cells to destroy the tumor cells. Immune checkpoint blockade can be combined with cancer vaccines. For example, seethe recent Amgen patent (US20150202290) on methods of treating melanoma by administering an immune checkpoint inhibitor in conjunction with a herpes simplex virus.

Adoptive cellular immunotherapy using chimeric antigen receptor (CAR) modified T cell technology has readied remarkable clinical achievements in recent years. More than 80 clinical trials have been registered on the ClinicalTrials.gov since December 2015. One of the featured studies showed that CAR-T against CD19 (CTL019) was efficient for sustained remissions in leukemia. Novartis reported the new CTL019 Phase II data demonstrating 93% (55 of 59 patients) complete remission in pediatric patients with r/r ALL at the 57th American Society of Hematology (ASH) Annual Meeting. Many CAR-T studies targeting different types of tumor antigens for different diseases are in progress. For example, BCMA, CD20, CD22, CD33, CD38, CEA, EGFR, GD2, HER2, IGF1R, mesothelin, PSMA, ROR1 and WT1 have been targeted. Miao et al reported use of CART directed to EGFRvIII in treating glioblastoma, one of the most lethal forms of cancer.

To date, therapeutic biologics especially monoclonal antibodies are generally produced by cells such as Chinese Hamster Ovarian cells (CHO), HEK293, NS0 and Sp2/0. CHO cell line has been used to produce almost 70% of all recombinant protein therapeutics, such as HUMIRA® (adalimumab), ENBREL® (etanercept), RITUXAN® (Rituximab), AVASTIN® (bevacizumab) and HERCEPTIN® trastuzumab). Ipilimumab and Lambrolizumab are also produced in CHO cell culture in the industry. After their production by complicated bio-process procedures, these biologics are purified and formulated as injection or infusion compositions for clinical use.

The disclosures of all publications, patents, patent applications and published patent applications referred to herein are hereby incorporated herein by reference in their entirety.

### BRIEF SUMMARY OF THE INVENTION

The present application provides pharmaceutical compositions comp rising engineered mammalian cells that express an immunomodulator, and methods of use thereof for treating cancer.

In one aspect of the present application, there is provided a pharmaceutical composition comprising: a) an engineered mammalian cell comprising a heterologous nucleic acid encoding an immunomodulator, wherein the heterologous nucleic acid is operably linked to a promoter; and b) a pharmaceutically acceptable excipient. In some embodiments, the heterologous nucleic acid is present in the genome of the engineered mammalian cell.

In some embodiments according to any of the pharmaceutical compositions described above, the engineered mammalian cell is a primary cell. In some embodiments, the engineered mammalian cell is derived from a cell line, such as a cell line selected from the group consisting of HEK293-6E cells, NK-92 cells, and Jurkat cells.

In some embodiments according to any of the pharmaceutical compositions described above, the engineered mammalian cell is an immune cell, such as a peripheral blood monocyte cell (PBMC), T cell, B cell, or NK cell. In some embodiments, the engineered mammalian cell further expresses a chimeric antigen receptor (CAR) or a recombinant T cell receptor (TCR). In some embodiments, the engineered mammalian cell comprises a vector comprising the heterologous nucleic acid encoding the immunomodulator and a second heterologous nucleic acid encoding the CAR or the TCR. In some embodiments, the second heterologous nucleic acid encoding the CAR or the TCR is operably linked to the promoter. In some embodiments, the promoter is inducible by the intracellular signaling domain of the chimeric antigen receptor or the recombinant T cell receptor. In some embodiments, the CAR or TCR comprises an intracellular signaling domain with an abolished or attenuated immune effector function. In some embodiments, the CAR is a truncated CAR. In some embodiments, the CAR does not comprise a primary intracellular signaling domain (such as CD3ζ). In some embodiments, the CAR comprises a nonfunctional or attenuated primary intracellular signaling domain (such as a mutant CD3ζ).

In some embodiments, the engineered mammalian cell is a stem cell, such as a hematopoietic stem cell, a mesenchymal stem cell, or an induced pluripotent stem cell (iPSC). In some embodiments, the engineered mammalian cell further expresses a chimeric antigen receptor (CAR) or a recombinant T cell receptor (TCR). In some embodiments, the promoter is inducible by the intracellular signaling domain of the chimeric antigen receptor or the recombinant T cell receptor.

In some embodiments according to any of the pharmaceutical compositions described above, the pharmaceutical composition further comprises a second cell, wherein the second cell is a mammalian immune cell expressing a chimeric antigen receptor or a recombinant T cell receptor.

In some embodiments according to any of the pharmaceutical compositions described above, the promote is an endogenous promoter. In some embodiments, the promoter is a heterologous promoter.

In some embodiments according to any of the pharmaceutical compositions described above, the promoter is a promoter inducible by an inducing condition. In some embodiments, the inducing condition is selected from the group consisting of: inducer, irradiation, temperature, redox state, tumor environment, and the activation state of the engineered mammalian cell. In some embodiments, the promoter is inducible by an endogenous activation signal of the engineered mammalian cell. In some embodiments, the promoter is a T cell activation-dependent promoter. In some embodiments, the promoter is inducible by an inducer, such as a small molecule, a polypeptide (for example, a polypeptide expressed by the engineered mammalian cell).

In some embodiments according to any of the pharmaceutical compositions described above, the engineered mammalian cell further expresses on its surface a targeting molecule recognizing a tumor antigen.

In some embodiments according to any of the pharmaceutical compositions described above, the immunomodulator is an immune checkpoint inhibitor. In some embodiments, the immune checkpoint inhibitor is an inhibitor of PD-1, PD-L1, PD-L2, CTLA-4, BLTA, TIM-3, or LAG-3. In some embodiments, the immunomodulator is an immunoactivator. In some embodiments, the immunoactivator is selected from the group consisting of IL-2, IL-7, IL-15, IL-21, IL-12, CCR4, CCR2b, Heparanase, CD137L, LEM, and Bcl-2.

In some embodiments according to any of the pharmaceutical compositions described above, the immunomodulator is a secreted protein. In some embodiments, the immunomodulator is an antibody. In some embodiments, the antibody is a single chain antibody. In some embodiments, the immunomodulator is a single-domain antibody. In some embodiments, the immunomodulator is a heavy chain-only antibody. In some embodiments, the immunomodulator is an Fc-containing antibody (such as full-length antibody).

In some embodiments according to any of the pharmaceutical compositions described above, the immunomodulator is an antibody comprising a heavy chain and a light chain. In some embodiments, the nucleic acid encoding the heavy chain and the nucleic acid encoding the light chain are operably linked to the same promoter. In some embodiments, the nucleic acid encoding the heavy chain and the nucleic acid encoding the light chain are operably linked to different promoters. In some embodiments, the promoter for the nucleic acid encoding the heavy chain and the promoter for the nucleic acid encoding the light chain can be simultaneously induced. In some embodiments, the promoter for the nucleic acid encoding the heavy chain and the promoter for the nucleic acid encoding the light chain can be sequentially induced. In some embodiments, the promoter for the nucleic acid encoding the heavy chain and the promoter for the nucleic acid encoding the light chain have a strength ratio of about 10:1 to about 1:10.

In some embodiments according to any of the pharmaceutical compositions described above, the engineered mammalian cell further comprises a second heterologous nucleic acid encoding a therapeutic protein, such as an immunomodulator, or a therapeutic protein that is not an immunomodulator. In some embodiments, the heterologous nucleic acid encoding the immunomodulator and the second heterologous nucleic acid encoding the therapeutic protein are operably linked to the same promoter. In some embodiments, the heterologous nucleic acid encoding the immunomodulator and the second heterologous nucleic acid encoding the therapeutic protein are operably linked to different promoters. In some embodiments, the engineered mammalian cell expresses the immunomodulator and two or more therapeutic proteins.

In some embodiments according to any of the pharmaceutical compositions described above, the engineered mammalian cell expresses the immunomodulator at a sufficiently high level such that the composition is therapeutically effective. In some embodiments, the engineered mammalian cell expresses the immunomodulator at a level of at least about 1 mg/L, including for example at least about any of 5 mg/L, 10 mg/L, 20 mg/L, 50 mg/L, 100 mL, 200 mg/L, 300 mg/L, 400 mg/L, 500 mg/L, 600 mg/L, 700 mg/L, 800 mg/L, 900 mg/L, 1 g/L, 20 g/L, 3 g/L, 4 g/L, 5 mg/L or 10 g/L.

In one aspect of the present application, there is provided a method of treating a cancer in an individual (such as a human individual), comprising administering to the individual an effective amount of any one of the pharmaceutical compositions described above. In some embodiments, the pharmaceutical composition is administered systemically, such as by infusion. In some embodiments, the pharmaceutical composition is locally administered to a site of tumor, for example, by injection.

In some embodiments according to any one of the methods of treating cancer described above, the method further comprises inducing the expression of the immunomodulator in the engineered mammalian cell.

In some embodiments according to any one of the methods of treating cancer described above, the cancer is a solid tumor. In some embodiments, the cancer is a liquid tumor.

In some embodiments according to any one of the methods of treating cancer described above, the engineered mammalian cell is obtained from the individual. In some embodiments, the engineered mammalian cell is allogenic to the individual.

In one aspect of the present application, there is provided a method of preparing any one of the pharmaceutical compositions described above, comprising introducing into a mammalian cell a vector comprising the heterologous nucleic acid encoding the immunomodulator. In some embodiments, the vector is a viral vector, such as a viral vector selected from the group consisting of a lentiviral vector, a retroviral vector, an adenoviral vector, an adeno-associated viral vector, a herpes simplex viral vector, and derivatives thereof. In some embodiments, the vector is introduced into the cell by electroporation.

In one aspect of the present application, there is provided a kit comprising: a) any one of the pharmaceutical compositions described above, and b) an instruction for using the pharmaceutical composition. In some embodiments, the kit further comprises c) a composition comprising a second mammalian immune cell expressing a chimeric antigen receptor or a recombinant T cell receptor.

These and other aspects and advantages of the present invention will become apparent from the subsequent detailed description and the appended claims. It is to be understood that one, some, or all of the properties of the various embodiments described herein may be combined to form other embodiments of the present invention.

### BRIEF DESCRIPTION OF THE DRAWINGS

FIG. 1 is a schematic view of one exemplary embodiment showing constitutive expression of an immune checkpoint inhibitor in an engineered mammalian cell, and blockade of an inhibitory immune checkpoint molecule expressed on a tumor cell.

FIG. 2 is a schematic view of one exemplary embodiment showing constitutive expression of an immune checkpoint inhibitor in an engineered mammalian cell, and blockade of an inhibitory immune checkpoint molecule expressed on the engineered mammalian cell and an unmodified immune cell.

FIG. 3 is a schematic view of one exemplary embodiment showing inducible expression of an immune checkpoint inhibitor in an engineered mammalian cell, and blockade of an inhibitory immune checkpoint molecule expressed on a tumor cell.

FIG. 4 is a schematic view of one exemplary embodiment showing inducible expression of an immune checkpoint inhibitor in an engineered mammalian cell, and blockade of an inhibitory immune checkpoint molecule expressed on the engineered mammalian cell and an unmodified immune cell.

FIG. 5 is a schematic view of one exemplary embodiment showing co-expression of an immune checkpoint inhibitor and a chimeric antigen receptor (CAR) by a mammalian engineered cell. Secretion of the immune checkpoint inhibitor and blockade of an inhibitory immune checkpoint molecule expressed on a tumor cell are controlled by activation of the CAR intracellular signaling domain downstream of tumor antigen recognition by the CAR molecules.

FIG. 6 is a schematic view of one exemplary embodiment showing co-expression of an immune checkpoint inhibitor and a chimeric antigen receptor (CAR) by a mammalian engineered cell. Secretion of the immune checkpoint inhibitor and blockade of an inhibitory immune checkpoint molecule expressed on the engineered mammalian cell and an unmodified immune cell are controlled by activation of the CAR intracellular signaling domain downstream of tumor antigen recognition by the CAR molecules.

FIG. 7A is a schematic view of an exemplary embodiment showing co-expression of an immune checkpoint inhibitor and a truncated anti-EGFR CAR by a mammalian engineered immune cell (such as T cell). Binding of the CAR to EGFR overexpressed on tumor cells result in site-specific expression and secretion of the immune checkpoint inhibitor.

FIG. 7B is a schematic view of an exemplary embodiment showing co-expression of an immune checkpoint inhibitor, a truncated anti-EGFR CAR by a mammalian engineered immune cell (such as T cell), and one or more immunoactivators, such as IL-7, IL-21, CCRs, and Bcl2. Binding of the CAR to EGFR overexpressed on tumor cells result in site-specific expression and secretion of the immune checkpoint inhibitor and the immunoactivators.

FIG. 8A shows anti-PD-1 and anti-CTLA-4 antibody expression driven by a hEF1α promoter in transduced primary human T cells.

FIG. 8B shows anti-PD-1 and anti-CTLA-4 antibody expression driven by a hEF1α promoter in transduced primary human B cells.

FIG. 8C shows anti-PD-1 and anti-CTLA-4 antibody expression driven by a hEF1α promoter in transduced primary human NK cells.

FIG. 9A shows anti-PD-1 expression driven by a TETON® promoter in transduced primary human T cells.

FIG. 9B shows anti-CTLA-4 antibody expression driven by a TETON® promoter in transduced primary human T cells.

FIG. 10A shows anti-PD-1 expression driven by an NFAT promoter in transduced primary human T cells.

FIG. 10B shows anti-CTLA-4 antibody expression driven by an NFAT promoter in transduced primary human T cells.

FIG. 11 shows anti-PD-1 expression driven by a temperature-controlled promoter in transduced primary human T cells.

FIG. 12A shows expression of PD-1 in reporter cell line Jurkat/NFAT.Luc-PD-1.

FIG. 12B shows expression of PD-L1 in reporter cell line CHO/PD-L1.

FIG. 12C shows expression of CTLA-4 in reporter cell line Jurkat/IL-2 promoter.Luc.CTLA-4.

FIG. 13A shows binding affinity of anti-PD-1 antibody to engineered reporter cell lines.

FIG. 13B shows binding affinity of anti-CTLA-4 antibody to engineered reporter cell lines.

FIG. 14 shows in vitro activity of anti-CTLA-4 antibody in a CTLA-4 reporter assay.

FIG. 15A shows expression of EGFRvIII on reporter cell line U87MG/vIII.Luc.PD-L1.

FIG. 15B shows expression of PD-L1 on reporter cell line U87MG/vIII.Luc.PD-L1.

FIG. 16A shows cytotoxicity of engineered T cells expressing an anti-EGFRvIII-CAR and/or anti-PD-1 antibody against U87MG/vIII-luc-PD-L1 tumor cells.

FIG. 16B shows IFN-gamma secretion by engineered T cells expressing an anti-EGFRvIII-CAR and/or anti-PD-1 antibody co-cultured with U87MG/vIII-luc-PD-L1 tumor cells.

FIG. 16C shows anti-PD-1 antibody expression by engineered T cells alone, or co-cultured with U87MG/vIII-luc-PD-L1 tumor cells.

FIG. 17 shows cytotoxicity of engineered T cells expressing an anti-BCMA-CAR and/or anti-PD-1 antibody against RPMI-8226/luc-PD-L1 tumor cells.

FIG. 18 shows cytotoxicity of engineered T cells expressing an anti-EGFRvIII-CAR and/or anti-CTLA-4 antibody against U87MG/vIII-luc-CD80/CD86 tumor cells.

FIG. 19A shows cytotoxicity of engineered T cells expressing various anti-NY-ESO-1-TCRs (LIT-001˜LIT-006) against U87MG/ESO1-luc-PD-L1 tumor cells.

FIG. 19B shows cytotoxicity of engineered T cells expressing an anti-NY-ESO-1-TCR (LIT-006) and/or anti-PD-1 antibody against U87M G/ESO1-luc-PD-L1 tumor cells.

FIG. 19C shows IFN-gamma secretion by engineered T cells expressing an anti-NY-ESO-1-TCR (LIT-006) and/or anti-PD-1 antibody co-cultured with U87MG/ESO1-luc-PD-L1 tumor cells.

FIG. 20 shows cytotoxicity of engineered T cells expressing an anti-NY-ESO-1-TCR (LIT-006) and/or anti-CTLA-4 antibody against U87MG/ESO1-luc-CD80/CD86 tumor cells.

FIG. 21A shows antibody expression levels by engineered T cells transduced with vectors encoding anti-HER2 antibody and/or anti-PD-1 antibody.

FIG. 21B shows antibody expression levels by engineered T cells transduced with vectors encoding anti-HER2 antibody and/or anti-CTLA-4 antibody.

FIG. 22A shows cytotoxicity of engineered T cells expressing anti-HER2 antibody and/or anti-PD-1 antibody against SK-BR-3/luc cells.

FIG. 22B shows cytotoxicity of engineered T cells expressing anti-HER2 antibody and/or anti-CTLA-4 antibody against SK-BR-3/luc cells.

FIG. 23 depicts various constructs encoding anti-EGFR CAR comprising mAb425 scFv. GSI054-GSI060 further encode an anti-PD-1 antibody. GSI055-GSI060 further encodes one or more immunoactivators, such as IL-7 or IL-21, CCR2b or CCR4, and/or Bcl2.

FIG. 24 shows results of an in vitro cytotoxicity assay of anti-EGFR CAR-T cells against A549-luc cells co-cultured for 7 days.

FIG. 25A shows cytotoxicity assay of truncated CAR-T cells against A549-luc cells co-cultured for 1 day.

FIG. 25B shows cytotoxicity assay of truncated CAR-T cells against A549-luc cells co-cultured for 3 days.

FIG. 25C shows cytotoxicity assay of truncated CAR-T cells against A549-luc cells co-cultured for 5 days.

FIG. 25D shows cytotoxicity assay of truncated CAR-T cells against A549-luc cells co-cultured for 7 days.

FIG. 26A shows anti-PD-1 expression by truncated CAR-T cells co-cultured with A549-luc cells for 3 days.

FIG. 26B shows IL-21 expression by truncated CAR-T cells co-cultured with A549-luc cells for 3 days.

FIG. 27A shows expression of CCR4 in GSI059 transduced T cells.

FIG. 27B shows express ion of CCR4 in GSI060 transduced T cells.

FIG. 27C shows expression of Bcl2 in GSI060 transduced T cells.

FIG. 28 shows cytotoxicity of GSI060 transduced primary T cells obtained from a cynomolgus monkey.

FIG. 29A shows body weight of cynomolgus monkeys infused with CAR-T.

FIG. 29B shows body temperature of cynomolgus monkeys infused with CAR-T.

FIG. 29C shows complete blood counts of cynomolgus monkey NHP#2 infused with CAR-T.

FIG. 29D shows serum chemistry of cynomolgus monkey NHP#2 infused with CAR-T.

FIG. 30A shows cytotoxicity of EGFRvIII CAR-T cells expressing an anti-PD-1 sdAb against U87MG/vIII-luc-PD-L1 tumor cells.

FIG. 30B shows cytotoxicity of EGFRvIII CART cells expressing an anti-CTLA-4 sdAb against U87M G/vIII-luc-CD80/CD86 tumor cells.

### DETAILED DESCRIPTION OF THE INVENTION

The present invention provides pharmaceutical compositions comprising an engineered mammalian cell comprising a heterologous nucleic acid encoding an immuno modulator, such as an immune checkpoint inhibitor. Unlike traditional pharmaceutical compositions comprising immunomodulators, the pharmaceutical compositions described herein can provide a controllable, localized, and cost-effective cell-based delivery system of immunomodulators to tumor cells. The pharmaceutical compositions of the present invention may further comprise a Chimeric Antigen Receptor (CAR) or a recombinant T cell receptor (TCR) expressed by either the engineered mammalian cell (such as an immune cell) or by a second cell. The combined functions of CAR or TCR activation and immunomodulator secretion in such two-component pharmaceutical compositions may reinforce each other in a positive feedback loop, thereby enhancing cytotoxicity of the engineered cells against tumor cells, while recruiting unmodified host immune cells to the tumor cells at the same time. The pharmaceutical compositions described herein are useful for providing an intensified and robust immunotherapy against cancer (such as solid tumor) to an individual in need thereof.

In one aspect of the present application, there is provided a pharmaceutical composition comprising: a) an engineered mammalian cell comprising a heterologous nucleic acid encoding an immunomodulator, wherein the heterologous nucleic acid is operably linked to a promoter; and b) a pharmaceutically acceptable excipient.

In some embodiments, there is provided a pharmaceutical composition comprising: a) an engineered mammalian cell (such as an immune cell or astern cell) comprising a heterologous nucleic acid encoding an immunomodulator, wherein the heterologous nucleic acid is operably linked to a promoter, wherein the engineered mammalian cell further expresses a chimeric antigen receptor (CAR) or a recombinant T cell receptor (TCR); and b) a pharmaceutically acceptable excipient.

In some embodiments, there is provided a pharmaceutical composition comprising: a) an engineered mammalian cell comprising a heterologous nucleic acid encoding an immunomodulator, wherein the heterologous nucleic acid is operably linked to a promoter; b) a second mammalian immune cell expressing a chimeric antigen receptor (CAR) or a recombinant T cell receptor (TCR); and c) a pharmaceutically acceptable excipient.

In another aspect of the present application, there is provided a method of treating a cancer in an individual, comprising administering to the individual an effective amount of a pharmaceutical composition comprising: a) an engineered mammalian cell comprising a heterologous nucleic acid encoding an immuno modulator, wherein the heterologous nucleic acid is operably linked to a promoter; and b) a pharmaceutically acceptable excipient.

In some embodiments, there is provided a method of treating a cancer in an individual, comprising administering to the individual an effective amount of a pharmaceutical composition comprising: a) an engineered mammalian cell (such as an immune cell or a stem cell) comprising a heterologous nucleic acid encoding an immunomodulator, wherein the heterologous nucleic acid is operably linked to a promoter, wherein the engineered mammalian cell further expresses a chimeric antigen receptor (CAR) or a recombinant T cell receptor (TCR); and b) a pharmaceutically acceptable excipient.

In some embodiments, there is provided a method of treating a cancer in an individual, comprising administering to the individual an effective amount of a pharmaceutical composition comprising: a) an engineered mammalian cell comprising a heterologous nucleic acid encoding an immunomodulator, wherein the heterologous nucleic acid is operably linked to a promoter; b) a second mammalian immune cell expressing a chimeric antigen receptor (CAR) or a recombinant T cell receptor (TCR); and c) a pharmaceutically acceptable excipient.

In some embodiments, there is provided a method of treating a cancer in an individual, comprising: a) administering to the individual an effective amount of a pharmaceutical composition comprising an engineered mammalian cell comprising a heterologous nucleic acid encoding an immunomodulator, wherein the heterologous nucleic acid is operably linked to a promoter; b) administering to the individual an effective amount of a pharmaceutical composition comprising a second mammalian immune cell expressing a chimeric antigen receptor (CAR) or a recombinant T cell receptor (TCR).

Also provided are kits and articles manufacture useful for the methods described herein.

### I. Definitions

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. For example, an individual is successfully “treated” for cancer if one or more symptoms associated with cancer are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, and/or prolonging survival of individuals.

As used herein, “delaying progression of a disease” means to defer, hinder, slow, retard, stabilize, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

An “effective amount” is at least the minimum amount required to effect a measurable improvement of a particular disorder. An effective amount herein may vary according to factors such as the disease state, age, sex, and weight of the patient, and the ability of the antibody to elicit a desired response in the individual. An effective amount is also one in which any toxic or detrimental effects of the treatment are outweighed by the therapeutically beneficial effects. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder.

As used herein, “in conjunction with” refers to administration of one treatment modality in addition to another treatment modality. As such, “in conjunction with” refers to administration of one treatment modality before, during, or after administration of the other treatment modality to the individual.

A “subject” or an “individual” for purposes of treatment refers to any animal classified as a mammal, including humans, domestic and farm animals, and zoo, sports, or pet animals, such as dogs, horses, cats, cows, etc.

The term “antibody” herein is used in the broadest sense and specifically covers monoclonal antibodies (in eluding full length monoclonal antibodies), multispecific antibodies (e.g., bispecific antibodies), and antibody fragments so long as they exhibit the desired biological activity.

The terms “native antibody”, “full length antibody,” “intact antibody” and “whole antibody” are used herein interchangeably to refer to an antibody in its substantially intact form, not antibody fragments as defined below. The terms particularly refer to an antibody with heavy chains that contain an Fc region. Native antibodies are usually heterotetrameric glycoproteins of about 150,000 Daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The term “constant domain” refers to the portion of an immunoglobulin molecule having a more conserved amino acid sequence relative to the other portion of the immunoglobulin, the variable domain, which contains the antigen binding site. The constant domain contains the C_{H}1, C_{H}2 and C_{H}3 domains (collectively, CH) of the heavy chain and the CHL (or CL) domain of the light chain.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various immune effector functions, such as participation of the antibody in antibody-dependent cellular toxicity (ADCC).

The “light chains” of antibodies (immunoglobulins) from any mammalian species can be assigned to one of two clearly distinct types, called kappa (“κ”) and lambda (“λ”), based on the amino acid sequences of their constant domains.

The term IgG “isotype” or “subclass” as used herein is meant any of the subclasses of immuno globulins defined by the chemical and antigenic characteristics of their constant regions.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, γ, ε, γ, and μ, respectively. The subunit structures and three-dimensional configurations of different classes of immuno globulins are well known and described generally in, for example, Abbas et al. Cellular and Mol. Immunology, 4th ed. (W.B. Saunders, Co., 2000). An antibody may be part of a larger fusion molecule, formed by covalent or non-covalent association of the antibody with one or more other proteins or peptides.

The terms “full length antibody,” “intact antibody” and “whole antibody” are used herein interchangeably to refer to an antibody in its substantially intact form, not antibody fragments as defined below. The terms particularly refer to an antibody with heavy chains that contain an Fc region.

“Antibody fragments” comprise a portion of an intact antibody, preferably comprising the antigen binding region thereof. In some embodiments, the antibody fragment described herein is an antigen-binding fragment. Examples of antibody fragments include Fab, Fab′, F(ab′)₂, and Fv fragments; diabodies; linear antibodies; single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Fv” is the minimum antibody fragment which contains a complete antigen-binding site. In one embodiment, a two-chain Fv species consists of a dimer of one heavy- and one light-chain variable domain in t, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three HVRs of each variable domain interact to define an antigen-binding site on the surface of the VH-VL di mer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv, see, e.g., Pluckthün, The Pharmacology of Monoclonal Antibodies. Springer Berlin Heidelberg, 1994. 269-315.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

The term “heavy chain-only antibody” or “HCAb” refers to a functional antibody, which comprises heavy chains, but lacks the light chains usually found in antibodies. Camelid animals (such as camels, llamas, or alpacas) are known to produce HCAbs.

The term “single-domain antibody” or “sdAb” refers to an antibody fragment consisting of a single monomeric variable antibody domain. In some cases, single domain antibodies are engineered from camelid HCAbs, and such sdAbs are referred herein as “nanobodies” or “V_{H}Hs”. Camelid sdAb is one of the smallest known antigen-binding antibody fragments (see, e.g., Hamers-Casterman et al., Nature 363:446-8 (1993); Greenberg et al., Nature 374:168-73 (1995); Hassanzadeh-Ghassabeh et al., Nanomedicine (Lord), 8:1013-26 (2013)).

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding poly peptide sequence was obtained by a process that includes the selection of a single target binding polypeptide sequence from a plurality of polypeptide sequences. For example the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to poly clonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the invention may be made by a variety of techniques, including, for example, the hybridoma method (e.g., Kohler and Milstein, Nature 256:495-97 (1975); Hon go et al., Hybridoma 14 (3): 253-260 (1995), Harlow et al., Antibodies: A Laboratory Manual, (Cold Spring Harbor Laboratory Press, 2nd ed. 1988); Hammerling et al., Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al, Nature 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004)), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad Sci. USA 90:2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lonberg et al., Nature 368: 856-859 (1994); Morrison, Nature 368: 812-813 (1994); Fishwild al., Nature Biotechnol. 14: 845-851 (1996); Neuberger, Nature Biotechnol. 14: 826 (1996); and Lonberg and Huszar, Intern. Rev. Immunol. 13:65-93 (1995)).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al., Proc. Natl. Acad. Sci. USA 81:6851-6855 (1984)). Chimeric antibodies include PRIMATTZED® antibodies wherein the antigen-binding region of the antibody is derived from an antibody produced by, e.g., immunizing macaque monkeys with the antigen of interest.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a HVR of the recipient are replaced by residues from a HVR of anon-human species (donor antibody) such as mouse, rat, rabbit, or nonhuman primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see, e.g., Jones et al., Nature 321:522-525 (1986); Riechmann a al., Nature 332:323-329 (1988); and Presta, Curr. Op. Struct. Biol. 2:593-596 (1992). See also, e.g., Vaswani and Hamilton, Ann. Allergy, Asthma & Immunol. 1:105-115 (1998); Harris, Biochem Soc. Transactions 23:1035-1038 (1995); Hurle and Gross, Curr. Op. Biotech 5:428433 (1994); and U.S. Pat. Nos. 6,982,321 and 7,087,409.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol. 227:381 (1991); Marks et al., J. Mol. Biol. 222:581 (1991). Also available for the preparation of human monoclonal antibodies are methods described in Cole et al., Monoclonal Antibodies and Cancer Therapy, Alan R Liss, 77 (1985); Boemer et al., J. Immunol. 147(1):86-95 (1991). See also van Dijk and van de Winkel, Curr. Opin. Pharmacol. 5: 368-74 (2001). Human antibodies can be prepared by administering the antigen to a transgenic animal that has bee modified to produce such antibodies in response to antigenic challenge, but whose endogenous loci have been disabled, e.g., immunized xenom ice (see, e.g., U.S. Pat. Nos. 6,075,181 and 6,150,584 regarding XENOMOUSE™ technology). See also, for example, Li et al., Proc. Natl. Acad. Sci. USA 103:3557-3562 (2006) regarding human antibodies generated via a human B-cell hybridoma technology.

As use herein, the term “binds”, “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of ≤1 μM, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding.

“Chimericantigen receptor” or “CAR” as used herein refers to genetically engineered receptors, which graft one or more antigen specificity onto cells, such as T cells. CARS are also known as “artificial T-cell receptors,” “chimeric T cell receptors,” or “chimeric immune receptors.” In some embodiments, the CAR comprises an extracellular variable domain of an antibody specific for a tumor antigen, and an intracellular signaling domain of a T cell or other receptors, such as one or more co-stimulatory signaling domains. “CAR-T” refers to a T cell that expresses a CAR.

“T cell receptor” or “TCR” as used herein refers to endogenous or recombinant T cell receptor comp rising an extracellular antigen binding domain that binds to a specific antigenic peptide bound in an MHC molecule. In some embodiments, the TCR comprises a TCRα polypeptide chain and a TCR polypeptide chain. In some embodiments, the TCR specifically binds a tumor antigen. “TCR-T” refers to a T cell that expresses a recombinant TCR.

The term “recombinant” refers to a biomolecule, e.g., a gene or protein, that (1) has been removed from its naturally occurring environment, (2) is not associated with all or a portion of a polynucleotide in which the gene is found in nature, (3) is operatively linked to a poly nucleotide which it is not linked to in nature, or (4) does not occur in nature. The term “recombinant” can be used in reference to cloned DNA isolates, chemically synthesized poly nucleotide analogs, or polynucleotide analogs that are biologically synthesized by heterologous systems, as well as proteins and/or mRNAs encoded by such nucleic acids.

The term “express” refers to translation of a nucleic acid into a protein Proteins may be expressed and remain intracellular, become a component of the cell surface membrane, or be secreted into extracellular matrix or medium.

The term “host cell” refers to a cell which can sup port the replication or expression of the expression vector. Host cells may be prokaryotic cells such as E. coli, or eukaryotic cells, such as yeast, insect cells, amphibian cells, or mammalian cells.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid.

The term“in vivo” refers to inside the body of the organism from which the cell is obtained. “Ex vivo” or “in vitro” means outside the body of the organism from which the cell is obtained.

The term “cell” includes the primary subject cell and its progeny.

As used herein, the term “immunomodulator” refers to any protein or peptide-based agent that has an effect (such as inhibitory or stimulatory effect) on the immune system.

As used herein, the term “immune checkpoint inhibitor” refers to a molecule that totally or partially reduces, inhibits or interferes with one or more checkpoint proteins, which can regulate T-cell activation and function.

As used herein, the term “immunoactivator” refers to a molecule that stimulates, activates, or increases the intensity of an immune response.

As used herein, the term “therapeutic protein” refers to any protein or peptide-based agent that has a therapeutic effect.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term“about X-Y” used herein has the same meaning as “about X to about Y.”

As used herein and in the appended claims, the singular forms “a,” “or,” and “the” include plural referents unless the context clearly dictates otherwise.

### II. Pharmaceutical Compositions

One aspect of the present invention provides a pharmaceutical composition comprising: a) an engineered mammalian cell comprising a heterologous nucleic acid encoding an immunomodulator, wherein the heterologous nucleic acid is operably linked to a promoter; and b) a pharmaceutically acceptable excipient. In some embodiments, the engineered mammalian cell is an immune cell. In some embodiments, the engineered mammalian cell is astern cell. In some embodiments, the promoter is inducible. In some embodiments, the immunomodulator is an immune checkpoint inhibitor. In some embodiments, the immunomodulator is an immunoactivator. In some embodiments, the immunomodulator is a secreted protein. In some embodiments, the immunomodulator is an antibody. In some embodiments, the engineered mammalian cell further expresses on its surface a targeting molecule recognizing a tumor antigen. In some embodiments, the engineered mammalian cell further comprises a second heterologous nucleic acid encoding a therapeutic protein (such as a second immunomodulator, or a therapeutic protein that is not an immunomodulator).

The pharmaceutical compositions of the present invention differ from compositions comprising cells for producing immunomodulators in many ways. For example, the immunomodulators expressed by the engineered mammalian cells of the present invention can be delivered to an individual in need thereof by directly administering the engineered mammalian cells to the individual, without isolating or purifying the immunomodulators from the engineered mammalian cells. In some embodiments, the pharmaceutical composition is suitable for administration to an individual, such as a human individual. In some embodiments, the pharmaceutical composition is suitable for injection. In some embodiments, the pharmaceutical composition is suitable for infusion. In some embodiments, the pharmaceutical composition is substantially free of cell culture medium. In some embodiments, the pharmaceutical composition is substantially free of endotoxins or allergenic proteins. In some embodiments, “substantially free” is less than about any of 10%, 5%, 1%, 0.1%, 0.01%, 0.001%, 1 ppm or less of total volume or weight of the pharmaceutical composition. In some embodiments, the pharmaceutical composition is free of mycoplasma, microbial agents, and/or communicable disease agents.

### Engineered Mammalian Cells

The pharmaceutical composition of the present applicant may comprise any number of the engineered mammalian cells. In some embodiments, the pharmaceutical composition comprises a single copy of the engineered mammalian cell. In some embodiments, the pharmaceutical composition comprises at least about any of 1, 10, 100, 1000, 10⁴, 10⁵, 10⁶, 10⁷, 10⁸ or more copies of the engineered mammalian cells. In some embodiments, the pharmaceutical composition comprises a single type of engineered mammalian cell. In some embodiments, the pharmaceutical composition comprises at least two types of engineered mammalian cells, wherein the different types, of engineered mammalian cells differ by their cell sources, cell types, expressed therapeutic proteins, immunomodulators, and/or promoters, etc.

The engineered mammalian cell can be derived from a variety of cell types and cell sources. Cells from any mammalian species, including, but not limited to, mice, rats, guinea pigs, rabbits, dogs, monkeys, and humans, are contemplated herein. In some embodiments, the engineered mammalian cell is a human cell. In some embodiments, the engineered mammalian cell is allogenic (i.e., from the same species, but different donor) as the recipient individual. In some embodiments, the engineered mammalian cell is autologous (i.e., the donor and the recipient are the same). In some embodiments, the engineered mammalian cell is syngeneic (i.e., the donor and the recipients are different individuals, but are identical twins).

In some embodiments, the engineered mammalian cell is derived from a primary cell. In some embodiments, the engineered mammalian cell is a primary cell isolated from an individual. In some embodiments, the engineered mammalian cell is propagated (such as proliferated and/or differentiated) from a primary cell isolated from an individual. In some embodiments, the primary cell is obtained from an epithelial, muscular, nervous, or connective tissue. In some embodiments, the primary cell is of the hematopoietic lineage. In some embodiments, the primary cell is obtained from the thymus. In some embodiments, the primary cell is obtained from the lymph or lymph nodes (such as tumor draining lymph nodes). In some embodiments, the primary cell is obtained from the spleen. In some embodiments, the primary cell is obtained from the bone marrow. In some embodiments, the primary cell is obtained from the blood, such as the peripheral blood. In some embodiments, the primary cell is a Peripheral Blood Mononuclear Cell (PBMC). In some embodiments, the primary cell is derived from the blood plasma. In some embodiments, the primary cell is derived from a tumor. In some embodiments, the primary cell is obtained from the mucosal immune system. In some embodiments, the primary cell is obtained from the skin. In some embodiments, the primary cell is obtained from a biopsy sample.

In some embodiments, the engineered mammalian cell is derived from a cell line. In some embodiments, the engineered mammalian cell is obtained from a commercial cell line. In some embodiments, the engineered mammalian cell is a cell line established from a primary cell isolated from an individual. In some embodiments, the engineered mammalian cell is propagated (such as proliferated and/or differentiated) from a cell line. In some embodiments, the cell line is mortal. In some embodiments, the cell line is immortalized. In some embodiments, the cell line is a tumor cell line, such as a leukemia or lymphoma cell line. In some embodiments, the cell line is a cell line derived from the PBMC. In some embodiments, the cell line is a stem cell line. In some embodiments, the cell line is selected from the group consisting of HEK293-6E cells, NK-92 cells, and Jurkat cells.

In some embodiments, the engineered mammalian cell is an immune cell. Exemplary immune cells useful for the present invention include, but are not limited to, dendritic cells (including immature dendritic cells and mature dendritic cells), T lymphocytes (such as naïve T cells, effector T cells, memory T cells, cytotoxic T lymphocytes, T helper cells, Natural Killer T cells, Treg cells, tumor infiltrating lymphocytes (TIL), and lyphokine-activated killer (LAK) cells), B cells, Natural Killer (NK) cells, monocytes, macrophages, neutrophils, granulocytes, and combinations thereof. Subpopulations of immune cells can be defined by the presence or absence of one or more cell surface markers known in the art (e.g., CD3, CD4, CD8, CD19, CD20, CD11c, CD123, CD56, CD34, CD14, CD33, etc.). In the cases that the pharmaceutical composition comprises a plurality of engineered mammalian immune cells, the engineered mammalian immune cells can be a specific subpopulation of an immune cell type, a combination of subpopulations of an immune cell type, or a combination of two or more immune cell types. In some embodiments, the immune cell is present in a homogenous cell population. In some embodiments, the immune cell is present in a heterogeneous cell population that is enhanced in the immune cell. In some embodiments, the engineered mammalian cell is a lymphocyte. In some embodiments, the engineered mammalian cell is not a lymphocyte. In some embodiments, the engineered mammalian cell is suitable for adoptive immunotherapy. In some embodiments, the engineered mammalian cell is a PBMC. In some embodiments, the engineered mammalian cell is an immune cell derived from the PBMC. In some embodiments, the engineered mammalian cell is a T cell. In some embodiments, the engineered mammalian cell is a CD4⁺ T cell. In some embodiments, the engineered mammalian cell is a CD8⁺ T cell. In some embodiments, the engineered mammalian cell is a B cell. In some embodiments, the engineered mammalian cell is an NK cell.

Thus, in some embodiments, there is provided a pharmaceutical composition comprising: a) an engineered mammalian (such as human) immune cell comp rising a heterologous nucleic acid encoding an immunomodulator, wherein the heterologous nucleic acid is operably linked to a promoter; and b) a pharmaceutically acceptable excipient. In some embodiments, the engineered mammalian immune cell is selected from a PBMC, a T cell, a B cell or an NK cell. In some embodiments, the promoter is inducible. In some embodiments, the immunomodulator is an immune checkpoint inhibitor. In some embodiments, the immunomodulator is an immunoactivator. In some embodiments, the immunomodulator is a secreted protein. In some embodiments, the immunomodulator is an antibody. In some embodiments, the engineered mammalian immune cell further expresses on its surface a targeting molecule recognizing a tumor antigen (such as CAR or TCR). In some embodiments, the engineered mammalian immune cell further comprises a second heterologous nucleic acid encoding a therapeutic protein (such as a second immunomodulator, or a therapeutic protein that is not an immunomodulator).

In some embodiments, there is provided a pharmaceutical composition comp rising: a) an engineered mammalian (such as human) T cell comprising a heterologous nucleic acid encoding an immunomodulator, wherein the heterologous nucleic acid is operably linked to a promoter; and b) a pharmaceutically acceptable excipient. In some embodiments, the engineered mammalian T cell is selected from a cytotoxic T cell, a helper T cell, a TIL, a LAK cell, a CAR-T or a TCR-T. In some embodiments, the promoter is inducible. In some embodiments, the promoter is a T cell activation-dependent promoter. In some embodiments, the immunomodulator is an immune checkpoint inhibitor. In some embodiments, the immunomodulator is an immunoactivator. In some embodiments, the immunomodulator is a secreted protein. In some embodiments, the immunomodulator is an antibody. In some embodiments, the engineered mammalian T cell further expresses on its surface a targeting molecule recognizing a tumor antigen (such as CAR or TCR). In some embodiments, the engineered mammalian T cell further comprises a second heterologous nucleic acid encoding a therapeutic protein (such as a second immunomodulator, or a therapeutic protein that is not an immunomodulator).

In some embodiments, there is provided a pharmaceutical composition comp rising: a) an engineered mammalian (such as human) PBMC comprising a heterologous nucleic acid encoding an immunomodulator, wherein the heterologous nucleic acid is operably linked to a promoter; and b) a pharmaceutically acceptable excipient. In some embodiments, the promoter is inducible. In some embodiments, the immunomodulator is an immune checkpoint inhibitor. In some embodiments, the immunomodulator is an immunoactivator. In some embodiments, the immunomodulator is a secreted protein. In some embodiments, the immunomodulator is an antibody. In some embodiments, the engineered mammalian PBMC further expresses on its surface a targeting molecule recognizing a tumor antigen (such as CAR or TCR). In some embodiments, the engineered mammalian PBMC further comprises a second heterologous nucleic acid encoding a therapeutic protein (such as a second immunomodulator, or a therapeutic protein that is not an immunomodulator).

In some embodiments, the engineered mammalian cell is a stem cell. In some embodiments, the stem cell is a totipotent stem cell. In some embodiments, the stem cell is a pluripotent stem cell. In some embodiments, the stem cell is a unipotent stem cell. In some embodiments, the stem cell is a progenitor cell. In some embodiments, the stem cell is an embryonic stem cell. In some embodiments, the stem cell is hematopoietic stem cell. In some embodiments, the stem cell is a mesenchymal stem cell. In some embodiments, the stem cell is an induced pluripotent stem cell (iPSC).

Thus, in some embodiments, there is provided a pharmaceutical composition comprising a an engineered mammalian (such as human) stem cell comprising a heterologous nucleic acid encoding an immuno modulator, wherein the heterologous nucleic acid is operably linked to a promoter; and b) a pharmaceutically acceptable excipient. In some embodiments, the engineered mammalian stem cell is selected from a hematopoietic stem cell a mesenchymal stem cell, or an iPSC. In some embodiments, the promoter is inducible. In some embodiments, the immunomodulator is an immune checkpoint inhibitor. In some embodiments, the immunomodulator is an immuno activator. In some embodiments, the immunomodulator is a secreted protein. In some embodiments, the immunomodulator is an antibody. In some embodiments, the engineered mammalian stem cell further expresses on its surface a targeting molecule recognizing a tumor antigen. In some embodiments, the engineered mammalian stem cell further comprises a second heterologous nucleic acid encoding a therapeutic protein (such as a second immunomodulator, or a therapeutic protein that is not an immunomodulator).

The engineered mammalian cell may comprise any number (such as any of 1, 2, 3, 4, 5, 10, 50, 100, 1000, or more) of the heterologous nucleic acid. In some embodiments, the engineered mammalian cell comprises a single copy of the heterologous nucleic acid. In some embodiments, the engineered mammalian cell comprises a plurality of copies of the heterologous nucleic acid. In some embodiments, the engineered mammalian cell comprises at least one additional heterologous nucleic acid, for example, a second heterologous nucleic acid encoding a second immuno modulator or a therapeutic protein that is not an immunomodulator, or a second heterologous nucleic acid encoding a reporter on the expression of a biomarker in the cell. In some embodiments, the engineered mammalian cell comprises two or more heterologous nucleic acids, each encoding a different therapeutic protein (such as immunomodulator or non-immunomodulator).

The heterologous nucleic acids described herein can be present in a heterologous gene expression cassette, which comprises one or more protein-coding sequences and optionally one or more promoters. In some embodiments, the heterologous gene expression cassette comprises a single protein-coding sequence. In some embodiments, the heterologous gene expression cassette comprises two or more protein-coding sequences driven by a single promoter (i.e., polycistronic). In some embodiments, the heterologous gene expression cassette further comprises one or more regulatory sequences (such as 5 ‘UTR, 3’ UTR, enhancer sequence, IRES, transcription termination sequence), recombination sites, one or more selection markers (such as antibiotic resistance gene, reporter gene, etc.), signal sequence, or combinations thereof. In some embodiments, the heterologous nucleic acid encoding the immuno modulator or the therapeutic protein comprises a signal sequence for secretion.

The heterologous nucleic acid may be transiently or stably incorporated in the engineered mammalian cell. In some embodiments, the heterologous nucleic acid is transiently expressed in the engineered mammalian cell. For example, the heterologous nucleic acid may be present in the nucleus of the engineered mammalian cell in an extrachromosomal array comprising the heterologous gene expression cassette. Heterologous nucleic acids may be introduced into the engineered mammalian using any transfection or transduction methods known in the art, including viral or non-viral methods. Exemplary non-viral transfection methods include, but are not limited to, chemical-based transfection, such as using calcium phosphate, dendrimers, liposomes, or cationic polymers (e.g., DEAE-dextran or polyethylenimine); non-chemical methods, such as electroporation, cell squeezing, sonoporation, optical transfection, impalefection, protoplast fusion, hydrodynamic delivery, or transposons; particle-based methods, such as using a gene gun, magnectofection or magnet assisted transfection, particle bombardment; and hybrid methods, such as nucleofection. In some embodiments, the heterologous nucleic acid is a DNA. In some embodiments, the heterologous nucleic acid is a RNA. In some embodiments, the heterologous nucleic acid is linear. In some embodiments, the heterologous nucleic acid is circular.

In some embodiments, the heterologous nucleic acid is present in the genome of the engineered mammalian cell. For example, the heterologous nucleic acid may be integrated into the genome of the engineered mammalian cell by any methods known in the art, including, but not limited to, virus-mediated integration, random integration, homologous recombination methods, and site-directed integration methods, such as using site-specific recombinase or integrase, transposase, Transcription activator-like effector nuclease (TALEN®), CRISPR/Cas9, and zinc-finger nucleases. In some embodiments, the heterologous nucleic acid is integrated in a specifically designed locus of the genome of the engineered mammalian cell. In some embodiments, the heterologous nucleic acid is integrated in an integration hotspot of the genome of the engineered mammalian cell. In some embodiments, the heterologous nucleic acid is integrated in a random locus of the genome of the engineered mammalian cell. In the cases that multiple copies of the heterologous nucleic acids are present in a single engineered mammalian cell, the heterologous nucleic acid may be integrated in a plurality of loci of the genome of the engineered mammalian cell.

### Immunomodulators

The engineered mammalian cell may express any number (such as any of 1, 2, 3, 4, 5, 6, or more) of immunomodulators. In some embodiments, the engineered mammalian cell comprises a heterologous nucleic acid encoding a single immunomodulator. In some embodiments, the engineered mammalian cell comprises one or more heterologous nucleic acids encoding at least two immunomodulators. In some embodiments, the heterologous nucleic acids encoding the at least two immunomodulators are operably linked to the same promoter. In some embodiments, the heterologous nucleic acids encoding the at least two immunomodulators are operably linked to different promoters.

Thus, in some embodiments, there is provided a pharmaceutical composition comprising: a) an engineered mammalian (such as human) cell comprising a heterologous nucleic acid encoding an immunomodulator, wherein the heterologous nucleic acid is operably linked to a promoter; and b) a pharmaceutically acceptable excipient. In some embodiments, the engineered mammalian cell is an immune cell (such as a PBMC, an NK cell, or a T cell). In some embodiments, the engineered mammalian cell is astern cell. In some embodiments, the promoter is inducible. In some embodiments, the immunomodulator is an immune checkpoint inhibitor. In some embodiments, the immunomodulator is an immunoactivator. In some embodiments, the immunomodulator is a secreted protein. In some embodiments, the immunomodulator is an antibody. In some embodiments, the engineered mammalian cell further expresses on its surface a targeting molecule recognizing a tumor antigen.

In some embodiments, there is provided a pharmaceutical composition comprising: a) an engineered mammalian (such as human) cell comprising one or more heterologous nucleic acids encoding at least two immunomodulators, wherein the heterologous nucleic acid encoding each immunomodulator is operably linked to a promoter; and b) a pharmaceutically acceptable excipient. In some embodiments, the engineered mammalian cell is an immune cell (such as a PBMC, an NK cell, or a T cell). In some embodiments, the engineered mammalian cell is a stem cell. In some embodiments, the heterologous nucleic acids encoding the at least two immunomodulators are operably linked to the same promoter. In some embodiments, the heterologous nucleic acids encoding the at least two immunomodulators are operably linked to different promoters. In some embodiments, the promoters are inducible. In some embodiments, the at least two immunomodulators comprise an immune checkpoint inhibitor. In some embodiments, the at least two immunomodulators comprise an immuno activator. In some embodiments, each of the at least two immunomodulators is a secreted protein. In some embodiments, each of the at least two immunomodulators is an antibody. In some embodiments, the engineered mammalian cell further expresses on its surface a targeting molecule recognizing a tumor antigen.

The immunomodulators contemplated herein are proteins or peptides. In some embodiments, the immunomodulator comprises a single polypeptide chain. In some embodiments, the immunomodulator comprises more than one (such as any of 2, 3, 4, or more) polypeptide chains. The polypeptide chain(s) of the immunomodulator may be of any length, such as at least about any of 10, 20, 50, 100, 200, 300, 500, or more amino acids long. In the cases of multi-chain immunomodulators, the nucleic acid sequences encoding the polypeptide chains may be operably linked to the same promoter or to different promoters.

In some embodiments, the immunomodulator is a secreted protein. In some embodiments, the immunomodulator is an antibody. Native antibodies, such as monoclonal antibodies, are immuno globulin molecules that are immunologically reactive with a particular antigen. The term “antibody” used herein includes genetically engineered forms, such as chimeric antibodies (e.g., humanized murine antibodies), heteroconjugate antibodies (e.g., bispecific antibodies), recombinant single chain Fv fragment (scFv), single-domain antibody, and heavy chain-only antibody. The term “antibody” also includes antigen binding forms of antibodies, such as Fab′, F(ab′)₂, scFv and V_{H}H. In some embodiments, the antibody is an agonistic antibody. In some embodiments, the antibody is an antagonistic antibody. In some embodiments, the antibody is a monoclonal antibody. In some embodiments, the antibody is a full-length antibody. In some embodiments, the antibody is an antigen-binding fragment selected from the group consisting of V_{H}, V_{L}, V_{NAR}, V_{H}H, Fab, Fab′, F(ab′)₂, Fv, minibody, scFv, sc(Fv)₂, tribody, tetrabody, BiTE, minibody, scFv-Fc, triabody, and other antigen-binding subsequences of the full length antibody or engineered combinations thereof. In some embodiments, the antibody is a human antibody, a humanized antibody, or a chimeric antibody. In some embodiments, the antibody is a monovalent antibody. In some embodiments, the antibody is a multivalent antibody, such as a divalent antibody or a tetravalent antibody. In some embodiments, the antibody is a bispecific antibody. In some embodiments, the antibody is a multispecific antibody. In some embodiments, the antibody is a single-domain antibody. In some embodiments, the antibody is a heavy chain-only antibody. In some embodiments, the antibody is a fusion protein comprising an antibody fragment (such as an Fc-containing fusion protein) or any other functional variants or derivatives of a full-length antibody.

In some embodiments, the immunomodulator is a single chain antibody. In some embodiments, the single chain antibody is a single-domain antibody. In some embodiments, the single chain antibody is an scFv. In some embodiments, the singe chain antibody is a bispecific single chain antibody, such as a tandem scFv or a BiTE. In some embodiments, the single chain antibody is a multispecific single chain antibody.

In some embodiments, the single chain antibody is a heavy chain-only antibody, such as a camelid antibody or a derivative thereof. In some embodiments, a pharmaceutical composition comprising an engineered mammalian cell co-expressing an immunomodulator (such as anti-PD-1 or anti-CTLA-4) that is a single-domain antibody and a CAR (or TCR) has more potent anti-tumor effect compared to a pharmaceutical composition comprising an engineered mammalian cell co-expressing an immunomodulator (such as anti-PD-1 or anti-CTLA-4) that is an IgG antibody and a CAR (or TCR). Without being bound by any theory or hypothesis, the small size, higher stability, and/or greater accessibility to buried epitopes associated with single-domain antibodies may contribute to the hirer efficacy of engineered cells co-expressing single-domain antibody immunomodulators and CAR (or TCR).

In some embodiments, the immunomodulator is an antibody comprising a heavy chain and a light chain. In some embodiments, the heavy chain comprises a V_{H} domain. In some embodiments, the heavy chain further comprises one or more constant domains, such as C_{H}1, C_{H}2, C_{H}3, or any combination thereof. In some embodiments, the light chain comprises a V_{L} domain. In some embodiments, the light chain further comprises one or more constant domains, such as C_{L}1, C_{L}2, C_{L}3, or any combination thereof. In some embodiments, the heavy chain and the light chain are connected to each other via a plurality of disulfide bonds. In some embodiments, the antibody comprises an Fc, such as an Fc fragment of the human IgG1, IgG2, IgG3, or IgG4. In some embodiments, the antibody does not comprise an Fc fragment. In some embodiments, the immunomodulator is a Fab.

The heavy chain polypeptide and the light chain polypeptide of multi-chain immunomodulatory antibodies are co-expressed in the engineered mammalian cell, either by a single heterologous nucleic acid, or by two heterologous nucleic acids. In some embodiments, the heavy chain polypeptide and the light chain polypeptide are expressed at equimolar ratio. In some embodiments, the heavy chain polypeptide and the light chain polypeptide are expressed at a ratio of about any of 10:1, 8:1, 6:1, 5:1, 4:1, 3:1, 2:1, 3:2, 4:3, 5:4, 1:1, 4:5, 3:4, 2:3, 1:2, 1:3, 1:4, 1:5, 1:6, 1:8, or 1:10.1n some embodiments, the heavy chain polypeptide and the light chain polypeptide are expressed at a ratio of any of about 1:10 to about 1:5, about 1:5 to about 1:3, about 1:4 to about 1:2, about 1:2 to about 1:1, about 1:1 to about 2:1, about 2:1 to about 4:1, about 3:1 to about 5:1, about 5:1 to about 10:1, about 1:2 to about 2:1, about 1:3 to about 3:1, about 1:5 to about 5:1, or about 1:10 to about 10:1. The optimal expression ratio between the heavy chain polypeptide and the light chain polypeptide may facilitate the antibody folding and assembly process. See, for example, Schlatter S et al., Biotechnol Prog. 21(1): 122-33 (2005).

The various expression ratio between the heavy chain polypeptide and the light chain polypeptide of a multi-chain immuno modulatory antibody may be achieved by manipulating the copy numbers of the heterologous nucleic acids and/or the nucleic acids encoding the heavy chain and the light chain, and/or the induction sequence and/or the strength of the promoters linked to the nucleic acids encoding the heavy chain and the chain. In some embodiments, the nucleic acid encoding the heavy chain and the nucleic acid encoding the light chain are operably linked to the same promoter. In some embodiments, the nucleic acid encoding the heavy chain and the nucleic acid encoding the light chain are operably linked to different promoters. In some embodiments, the promoter for the nucleic acid encoding the heavy chain and the promoter for the nucleic acid encoding the light chain can be simultaneously induced. In some embodiments, the promoter for the nucleic acid encoding the heavy chain and the promoter for the nucleic acid encoding the light chain can be sequentially induced. In some embodiments, the promoter for the nucleic acid encoding the heavy chain is induced prior to the induction of the promoter for the nucleic acid encoding the light chain. In some embodiments, the promoter for the nucleic acid encoding the heavy chain is induced after the induction of the promoter for the nucleic acid encoding the light chain. In some embodiments, the promoter for the nucleic acid encoding the heavy chain and the promoter for the nucleic acid encoding the light chain have a strength ratio of about any of 10:1, 8:1, 6:1, 5:1, 4:1, 3:1, 2:1, 3:2, 4:3, 5:4, 1:1, 4:5, 3:4, 2:3, 1:2, 1:3, 1:4, 1:5, 1:6, 1:8, or 1:10. In some embodiments, the promoter for the nucleic acid encoding the heavy chain and the promoter for the nucleic acid encoding the light chain have a strength ratio of any of about 1:10 to about 1:5, about 1:5 to about 1:3, about 1:4 to about 1:2, about 1:2 to about 1:1, about 1:1 to about 2:1, about 2:1 to about 4:1, about 3:1 to about 5:1, about 5:1 to about 10:1, about 1:2 to about 2:1, about 1:3 to about 3:1, about 1:5 to about 5:1, or about 1:10 to about 10:1.

Thus, in some embodiments, there is provided a pharmaceutical composition comprising a) an engineered mammalian (such as human) cell comprising a heterologous nucleic acid encoding an immunomodulatory antibody, wherein the heterologous nucleic acid is operably linked to a promoter; and b) a pharmaceutically acceptable excipient. In some embodiments, the engineered mammalian cell is an immune cell (such as a PBMC, an NK cell, or a T cell). In some embodiments, the engineered mammalian cell is a stem cell. In some embodiments, the promoter is inducible. In some embodiments, the immunomodulatory antibody is an immune checkpoint inhibitor. In some embodiments, the immunomodulatory antibody is an immunoactivator. In some embodiments, the immunomodulatory antibody is a single chain antibody (such as a single-domain antibody, an scFv, or a heavy chain-only antibody). In some embodiments, the immunomodulator antibody comprises a heavy chain and alight chain. In some embodiments, the engineered mammalian cell further expresses on its surface a targeting molecule recognizing a tumor antigen. In some embodiments, the engineered mammalian cell further comprises a second heterologous nucleic acid encoding a therapeutic protein (such as a second immunomodulator, or a therapeutic protein that is not an immunomodulator).

The immunomodulator expressed by the heterologous nucleic acid include any protein or peptide-based agent that modulates (such as inhibits or activates) the immune system. Immunomodulators can target specific molecules, such as the checkpoint molecules, or non-specifically modulate the immune response. Activators can include molecules that activate antigen presenting cells to stimulate the cellular immune response. For example, activators can be immunostimulant peptides. Activators can include, but are not limited to, agonists of toll-like receptors TLR-2, 3, 4, 6, 7, 8, or 9, granulocyte macrophage colony stimulating factor (GM-CSF), TNF, CD40L, CD-28, FLT-3 ligand, or cytokines such as IL-1, IL-2, IL-4, IL-7, IL-12, IL-15, or IL-21. Activators can include agonists of activating receptors (including co-stimulatory receptors) on T cells, such as an agonist (e.g., agonistic antibody) of CD-28, OX40, GITR, CD137, CD27, CD40, or HVEM. Activators can also include proteins that inhibit the activity of an immune suppressor, such as an inhibitor of the immune suppressors IL-10, IL-35, TGF-β, IDO, or inhibit the activity of an immune checkpoint such as an antagonist (e.g., antagonistic antibody) of CTLA-4, PD-1, PD-L1, PD-L2, LAG-3, B7-1, B7-H3, B7-H4, BTLA, VISTA, KIR, A2aR, or TIM-3. Activators can also include co-stimulatory molecules such as CD40, CD80, or CD86. Immunomodulators can also include agents that downregulate the immune system such as antibodies against IL-12p 70, antagonists of toll-like receptors TLR-2, 3, 4, 5, 6, 8, or 9, or general suppressors of immune function. These agents (e.g., activators, or downregulators) can be combined to achieve an optimal immune response. In some embodiments, the immunomodulator is a cytokine. In some embodiments, the immunomodulator is a chemokine.

Immunomodulators of particular interest in the present invention include modulators (such as inhibitors and activators) of the immune checkpoint proteins. Immune checkpoints are molecules in the immune system that either turn up (stimulatory molecules) or turn down a signal (inhibitory molecules). Immune checkpoint proteins regulate and maintain self-tolerance and the duration and amplitude of physiological immune responses. Stimulatory checkpoint molecules include, but are not limited to, CD27, CD40, OX40, GITR and CD137, which belong to tumor necrosis factor (TNF) receptor superfamily, as well as CD-28 and ICOS, which belong to the B7-CD-28 superfamily. Inhibitory checkpoint molecules include, but are not limited to, program death 1 (PD-1), Cytotoxic T-Lymphocyte-Associated protein 4 (CTLA-4), Lymphocyte Activation Gene-3 (LAG-3), T-cell Immunoglobulin domain and Mucin domain 3 (TIM-3), V-domain Ig suppressor of T cell activation (VISTA), B7-H3, B7-H4, B and T Lymphocyte Attenuator (BTLA), Indoleamine 2,3-dioxygenase (MO), Immunoglobulin like Receptor (KIR), adenosine A2A receptor, and ligands thereof. Numerous checkpoint proteins have been studied extensively, such as CTLA-4 and its ligands CD80 and CD86, and PD-1 with its ligands PD-L1 and PD-L2 (See, for example, Pardoll, Nature Reviews Cancer 12: 252-264 (2012)). The immunomodulators can be antibodies, natural ligands, or engineered proteins that specifically bind to the immune checkpoint molecule.

In some embodiments, the engineered mammalian cell expresses a single immunomodulator, such as a single immuno activator or a single immune checkpoint inhibitor. In some embodiments, the engineered mammalian cell expresses at least two immunomodulators. In some embodiments, the at least two immunomodulators are immunoactivators. In some embodiments, the at least two immunomodulators are immune checkpoint inhibitors. In some embodiments, the at least two immunomodulators comprise both immunoactivators and immune checkpoint inhibitors. In some embodiments, the at least two immunomodulators are expressed by the same heterologous nucleic acid. In some embodiments, the at least two immunomodulators are expressed by different heterologous nucleic acids, for example, each immunomodulator is expressed by a different heterologous nucleic acid.

In some embodiments, the immunomodulator is an immuno activator. Immunoactivators contemplated herein include, but are not limited to, activators of the stimulatory checkpoint molecules. In some embodiments, the immunoactivator is a natural or engineered ligand of a stimulatory immune checkpoint molecule, including, for example, ligands of OX40 (e.g., OX40L), ligands of CD-28 (e.g., CD80, CD86), ligands of ICOS (e.g., B7RP1), ligands of 4-1BB (e.g., 4-1BBL, Ultra4-1BBL), ligands of CD27 CD70), ligands of CD40 (e.g., CD40L), and ligands of TCR (e.g., MHC class I or class II molecules, IMCgp 100). In some embodiments, the immunoactivator is a secreted protein. In some embodiments, the immunoactivator is an antibody (such as an agonist antibody) selected from the group consisting of anti-CD-28 (e.g., TGN-1412), anti-OX40 (e.g., MED16469, MEDI-0562), anti-ICOS (e.g., MEDI-570), anti-GITR (e.g., TRX518, INBRX-110, NOV-120301), anti-4-1BB (e.g., BMS-663513, PF-05082566), anti-CD27 (e.g., BION-1402, Varlilumab and hCD27.15), anti-CD40 (e.g., CP870,893, BI-655064, BMS-986090, APX005, APX005M), anti-CD3 (e.g., blinatumomab, muromonab), and anti-HVEM.

In some embodiments, the immunomodulator is an immune checkpoint inhibitor. The term “immune checkpoint inhibitor” refers to molecules that totally or partially reduce, inhibit or interfere with one or more inhibitory checkpoint proteins, which can regulate T-cell activation and function. In some embodiments, the immune checkpoint inhibitor targets T cells. In some embodiments, the immune checkpoint inhibitor targets tumor cells. For example in some cases, tumor cells can turn off activated T cells, when they attach to specific T-cell receptors. However, immune checkpoint inhibitors may prevent tumor cells from attaching to T cells so that T cells stay activated (see, for example, Howard West, JAMA Oncol. 1(1):115 (2015)). In some embodiments, the immune checkpoint inhibitor is a natural or engineered ligand of an inhibitory immune checkpoint molecule, including, for example, ligands of CTLA-4 (e.g., B7.1, B7.2), liginds of TIM-3 (e.g., Galectin-9), ligands of A2a Receptor (e.g., adenosine, Regadenoson), ligands of LAG-3 (e.g., MHC class I or MHC class II molecules), ligands of BTLA (e.g., HVEM, B7-H4), ligands of KIR (e.g., MHC class I or MHC class II molecules), liginds of PD-1 (e.g., PD-L1, PD-L2), ligands of IDO (e.g., NKTR-218, Indoximod, NLG919), and ligands of CD47 (e.g., SIRP-alpha receptor). In some embodiments, the immune checkpoint inhibitor is secreted. In some embodiments, the immune checkpoint inhibitor is an antibody (such as antagonist antibody) that targets an inhibitory immune checkpoint protein selected from the group consisting of anti-CTLA-4 (e.g., Ipilimumab, Tremelimumab, KAHR-102), anti-TIM-3 (e.g., F38-2E2, ENUM005), anti-LAG-3 (e.g., BMS-986016, IMP701, IMP321, C9B7W), anti-KIR (e.g., Lirilumab and IPH2101), anti-PD-1 (e.g., Nivolumab, Pidilizumab, Pembrolizumab, BMS-936559, atezolizumab, Lambrolizumab, MK-3475, AMP-224, AMP-514, STI-A1110, TSR-042), anti-PD-L1 (e.g., KY-1003 (EP20120194977), MCLA-145, RG7446, BMS-936559, MEDI-4736, MSB0010718C, AUR-012, STI-A1010, PCT/US2001/020964, MPDL3280A, AMP-224, Dapirolizumabpegol (CDP-7657), MEDI-4920), anti-CD73 (e.g., AR-42 (OSU-HDAC42, HDAC-42, AR42, AR 42, OSU-HDAC 42, OSU-HDAC-42, NSC D736012, HDAC-42, HDAC 42, HDAC42, NSCD736012, NSC-D736012), MEDI-9447), anti-B7-H3 (e.g., MGA271, DS-5573a, 8H9), anti-CD47 (e.g., CC-90002, TTI-621, VIST-007), anti-BTLA, anti-VISTA, anti-A2aR, anti-B7-1, anti-B7-H4, anti-CD52 (such as alemtuzumab), anti-IL-10, anti-IL-35, and anti-TGF-β (such as Fresolumimab). In some embodiments, the immune checkpoint inhibitor is an inhibitor of an inhibitory checkpoint molecule selected from the group consisting of PD-1, PD-L1, PD-L2, CTLA-4, BLTA, TIM-3, and LAG-3.

In some embodiments, the immune checkpoint inhibitor is an inhibitor of CTLA-4. In some embodiments, the inhibitor of CTLA-4 is an anti-CTLA-4 antibody. In some embodiments, the anti-CTLA-4 antibody is Ipilimumab. Ipilimumab is an anti-CTLA-4 monoclonal antibody (trade name YERVOY®, formerly known as MDX-010 and MDX-101), which was approved by US FDA in March 2011 to treat patients with late-stage melanoma that has spread, or cannot be removed by surgery. This mAb drug has also shown promising response in clinical trials for the treatment of non-small cell lung carcinoma (NSCLC), small cell lung cancer (SCLC), bladder cancer and metastatic hormone-refractory prostate cancer.

Thus, in some embodiments, there is provided a pharmaceutical composition comprising: a) an engineered mammalian (such as human) cell comprising a heterologous nucleic acid encoding an inhibitor of CTLA-4 (such as an anti-CTLA-4 antibody, for example, Ipilimumab), wherein the heterologous nucleic acid is operably linked to a promoter; and b) a pharmaceutically acceptable excipient. In some embodiments, the engineered mammalian cell is an immune cell (such as a PBMC, an NK cell, or a T cell). In some embodiments, the engineered mammalian cell is a stem cell. In some embodiments, the promoter is inducible. In some embodiments, the engineered mammalian cell further expresses on its surface a targeting molecule recognizing a tumor antigen. In some embodiments, the engineered mammalian cell further comprises a second heterologous nucleic acid encoding a therapeutic protein (such as a second immunomodulator that is not an inhibitor of CTLA-4, or a therapeutic protein that is not an immunomodulator).

In so me embodiments, the immune checkpoint inhibitor is an inhibitor of PD-1. In some embodiments, the inhibitor of PD-1 is an anti-PD-1 antibody. In some embodiments, the anti-PD-1 antibody is Lambrolizumab. Lambrolizumab (also called Pembrolizumab or M K-3475, with trade name KEYTRUDA®) is a humanized anti-PD-1 IgG4 mAb approved by the US FDA on Sep. 4, 2014. This drug was initially used in treating metastatic melanoma. The US FDA approved pembrolizumab on Oct. 2, 2015, for the treatment of metastatic non-small cell lung cancer in patients whose tumors express PD-L1 and who have failed treatment with other chemotherapeutic agents.

Thus, in some embodiments, there is provided a pharmaceutical composition comprising a) an engineered mammalian (such as human) cell comprising a heterologous nucleic acid encoding an inhibitor of PD-1 (such as an anti-PD-1 antibody, for example, Lambrolizumab), wherein the heterologous nucleic acid is operably linked to a promoter; and b) a pharmaceutically acceptable excipient. In some embodiments, the engineered mammalian cell is an immune cell (such as a PBMC, an NK cell, or a T cell). In some embodiments, the engineered mammalian cell is a stem cell. In some embodiments, the promoter is inducible. In some embodiments, the engineered mammalian cell further expresses on its surface a targeting molecule recognizing a tumor antigen. In some embodiments, the engineered mammalian cell further comprises a second heterologous nucleic acid encoding a therapeutic protein (such as a second immunomodulator that is not an inhibitor of PD-1, or a therapeutic protein that is not an immunomodulator).

### Other Therapeutic Proteins

In some embodiments, the engineered mammalian cell further comprises a second heterologous nucleic acid encoding a therapeutic protein Therapeutic proteins contemplated herein include any protein or polypeptide-based agents that have a therapeutic effect. Therefore, immunomodulators are considered as a class of therapeutic proteins. The engineered mammalian cell may express any number (such as any of 1, 2, 3, 4, 5, 6, or more) of therapeutic proteins in addition to the immunomodulator. In some embodiments, the therapeutic protein is an immunomodulator. In some embodiments, the therapeutic protein is not an immunomodulator. In some embodiments, the engineered mammalian cell expresses the immunomodulator and two or more therapeutic proteins, including two or more additional immunomodulators, two or more therapeutic proteins that are not immunomodulators, or a combination of additional immunomodulator(s) and therapeutic protein(s) that are not immunomodulators.

The nucleic acid encoding the immunomodulator and the nucleic acid encoding the additional therapeutic protein (including immunomodulator and non-immunomodulators) may be driven by the same or different promoters. In some embodiments, the heterologous nucleic acid encoding the immunomodulator and the second heterologous nucleic acid encoding the therapeutic protein are operably linked to the same promoter (for example, in a polycistronic coding sequence). In some embodiments, wherein the heterologous nucleic acid is a polycistronic coding sequence encoding multiple proteins (such as immunomodulator, therapeutic protein, chimeric effector molecule, etc.), a nucleic acid sequence encoding a self-cleaving peptide, such as 2A peptides, for example, foot-and-mouse disease virus F2A, equine rhinitis A virus E2A, Thosea asigna virus T2A, or porcine teschovirus-1 P2A, can be disposed between sequencing encoding two different proteins. In some embodiments, the heterologous nucleic acid encoding the immunomodulator and the second heterologous nucleic acid encoding the therapeutic protein are operably linked to different promoters. Control by different promoters may allow differential expression levels, timing, and induction conditions for the immunomodulator and the additional therapeutic protein. In some embodiments, the heterologous nucleic acid encoding the immunomodulator and the second heterologous nucleic acid encoding the therapeutic protein are introduced to the engineered mammalian cell via the same heterologous gene expression cassette. In some embodiments, the heterologous nucleic acid encoding the immuno modulator and the second heterologous nucleic acid encoding the therapeutic protein are introduced to the engineered mammalian cell via separate heterologous gene expression cassettes.

Thus, in some embodiments, there is provided a pharmaceutical composition comprising a an engineered mammalian (such as human) cell comprising a heterologous nucleic acid encoding an immunomodulator, and a second heterologous nucleic acid encoding a therapeutic protein that is not an immunomodulator, wherein the heterologous nucleic acid encoding the immuno modulator is operably linked to a promoter; and b) a pharmaceutically acceptable excipient. In some embodiments, the engineered mammalian cell is a stem cell. In some embodiments, the promoter is inducible. In some embodiments, the immunomodulator is an immune checkpoint inhibitor (such as an inhibitor of CTLA-4 or an inhibitor of PD-1). In some embodiments, the immunomodulator is an immunoactivator. In some embodiments, the immunomodulator is a secreted protein. In some embodiments, the immunomodulator is an antibody. In some embodiments, the engineered mammalian cell further expresses on its surface a targeting molecule recognizing a tumor antigen. In some embodiments, the second heterologous nucleic acid encoding the therapeutic protein is operably linked to the promoter. In some embodiments, the second heterologous nucleic acid encoding the therapeutic protein is operably linked to a promoter that is different from the promoter operably linked to the heterologous nucleic acid encoding the immuno modulator.

In some embodiments, there is provided a pharmaceutical composition comp rising: a) an engineered mammalian (such as human) cell comprising a heterologous nucleic acid encoding an immunomodulator, wherein the heterologous nucleic acid encoding the immunomodulator is operably linked to a promoter, wherein the engineered mammalian cell further expresses two or more therapeutic proteins; and b) a pharmaceutically acceptable excipient. In some embodiments, the engineered mammalian cell is a stem cell. In some embodiments, the promoter is inducible. In some embodiments, the immunomodulator is an immune checkpoint inhibitor (such as an inhibitor of CTLA-4 or an inhibitor of PD-1). In some embodiments, the immunomodulator is an immunoactivator. In some embodiments, the immunomodulator is a secreted protein. In some embodiments, the immunomodulator is an antibody. In some embodiments, the engineered mammalian cell further expresses on its surface a targeting molecule recognizing a tumor antigen. In some embodiments, the two or more therapeutic proteins are not immunomodulators. In some embodiments, the two or more therapeutic proteins comprise one or more immunomodulators. In some embodiments, the two or more therapeutic proteins are each encoded by a heterologous nucleic acid operably linked to a promoter. In some embodiments, the promoters for the two or more therapeutic proteins are the same. In some embodiments, the promoters for the two or more therapeutic proteins are different. In some embodiments, one or more of the promoters for the two or more therapeutic proteins are the same as the promoter for the immunomodulator.

Any therapeutic protein known in the art may be expressed by the heterologous nucleic acid. Therapeutic proteins contemplated herein may have any one or more of the following functions: (1) replacing a protein that is deficient or abnormal; (2) augmenting an existing pathway; (3) providing a novel function or activity; (4) interfering with a molecule or organism; and (5) delivering other compounds or proteins.

In some embodiments, the therapeutic protein is an enzyme. In some embodiments, the therapeutic protein is a regulatory protein. In some embodiments, the therapeutic protein is a signaling protein. In some embodiments, the therapeutic protein targets a cell surface molecule. In some embodiments, the therapeutic protein is a ligand of a cell surface molecule. In some embodiments, the therapeutic protein is an inhibitor of a cell surface molecule. In some embodiments, the therapeutic protein is an activator of a cell surface molecule. In some embodiments, the therapeutic protein is a cell surface molecule. In some embodiments, the therapeutic protein is a chemotherapeutic agent. In some embodiments, the therapeutic protein specifically binds to a tumor antigen.

In some embodiments, the therapeutic protein is not an immunomodulator. Non-immunomodulator therapeutic proteins that are of particular interest in the present application are anti-cancer agents, such as chemotherapeutic antibodies. Chemotherapeutic antibodies contemplated herein include, but are not limited to, alemtuzumab, bevacizumab; cetuximab; panitumumab, rituximab, pertuzumab, trastuzumab, tositumomab, apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozopmicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories). In some embodiments, the therapeutic protein is an anti-HER2 antibody. In some embodiments, the anti-HER2 antibody binds to HER2 and inhibits cell proliferation or growth of HER2′ cancer cells. In some embodiments, the anti-HER2 antibody binds to HER2 and inhibits dimerization of HER2 with other HER receptors. In some embodiments, the anti-HER2 antibody is trastuzumab or pertuzumab. In some embodiments, the anti-HER2 antibody is not trastuzumab or pertuzumab.

The therapeutic proteins contemplated herein may have any of the molecular properties described above for the immunomodulators. In some embodiments, the therapeutic protein is secreted. For example, the therapeutic protein may be an antibody, including full length antibody, single chain antibody, single-domain antibody, heavy chain-only antibody, scFv, single-domain antibody (such as V_{H}H), and antibody fragments comprising a heavy chain and a light chain (such as Fab). The heterologous nucleic acids encoding the therapeutic protein and the promoters for the therapeutic protein may also have any of the properties described herein for those of the immunomodulators.

### Promoter

The heterologous nucleic acid encoding the immunomodulator, the cell surface molecule (CAR or TCR), or any other therapeutic protein described herein is operably linked to a promoter. In some embodiments, each of the immunomodulator, the cell surface molecule (such as CAR or TCR), and the other therapeutic protein is driven by a different promoter. In some embodiments, the immunomodulator, the cell surface molecule (such as CAR or TCR), and the other therapeutic protein are driven by the same promoter.

In some embodiments, the promoter is an endogenous promoter. For example the nucleic acid encoding the immunomodulator (or other therapeutic proteins described herein) may be knocked-in to the genome of the engineered mammalian cell downstream of an endogenous promoter using any methods known in the art, such as CRISPR/Cas9 method. In some embodiments, the endogenous promoter is a promoter for an abundant protein, such as beta-actin. In some embodiments, the endogenous promoter is an inducible promoter, for example, inducible by an endogenous activation signal of the engineered mammalian cell. In some embodiments, wherein the engineered mammalian c ell is a T cell, the promoter is a T cell activation-dependent promoter (such as an IL-2 promoter, an NFAT promoter, or an NFκB promoter).

In some embodiments, the promoter is a heterologous promoter.

Varieties of promoters have been explored for gene expression in mammalian cells, and any of the promoters known in the art may be used in the present invention. Promoters may be roughly categorized as constitutive promoters or regulated promoters, such as inducible promoters. In some embodiments, the heterologous nucleic acid encoding the immunomodulator is operably linked to a constitutive promoter. In some embodiments, the heterologous nucleic acid encoding the immunomodulator is operably linked to an inducible promoter. In some embodiments, a constitutive promoter is operably linked to the nucleic acid encoding a first therapeutic protein (such as the immunomodulator), and an inducible promoter is operably linked to a nucleic acid encoding a second therapeutic protein (such as a non-immunomodulator). In some embodiments, a first inducible promoter is operably linked to a nucleic acid encoding a first therapeutic protein (such as the immunomodulator) or polypeptide chain, and an second inducible promoter is operably linked to a nucleic acid encoding a second therapeutic protein (such as a non-immuno modulator) or polypeptide chain. In some embodiments, the first inducible promoter is inducible by a first inducing condition, and the second inducible promoter is inducible by a second inducing condition. In some embodiments, the first inducing condition is the same as the second inducing condition. In some embodiments, the first inducible promoter and the second inducible promoter are induced simultaneously. In some embodiments, the first inducible promoter and the second inducible promoter are induced sequentially, for example, the first inducible promoter is induced prior to the second inducible promoter, or the first inducible promoter is induced after the second inducible promoter.

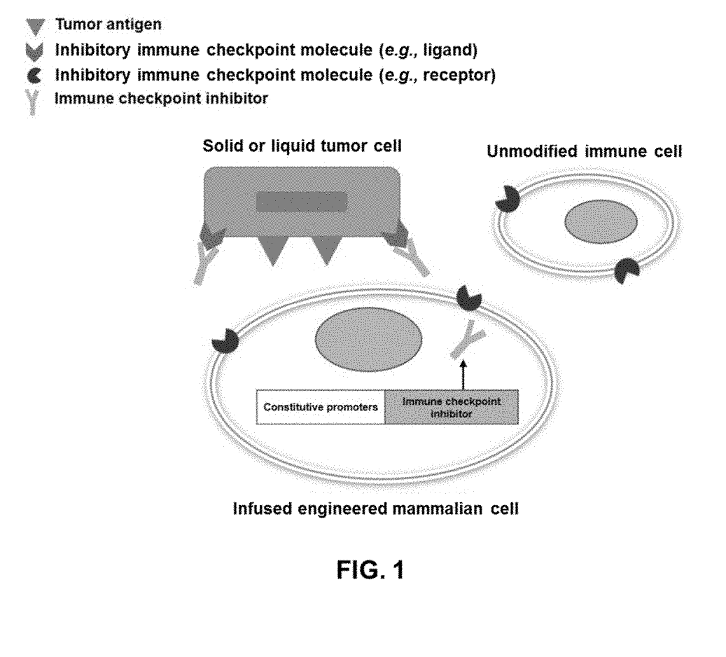

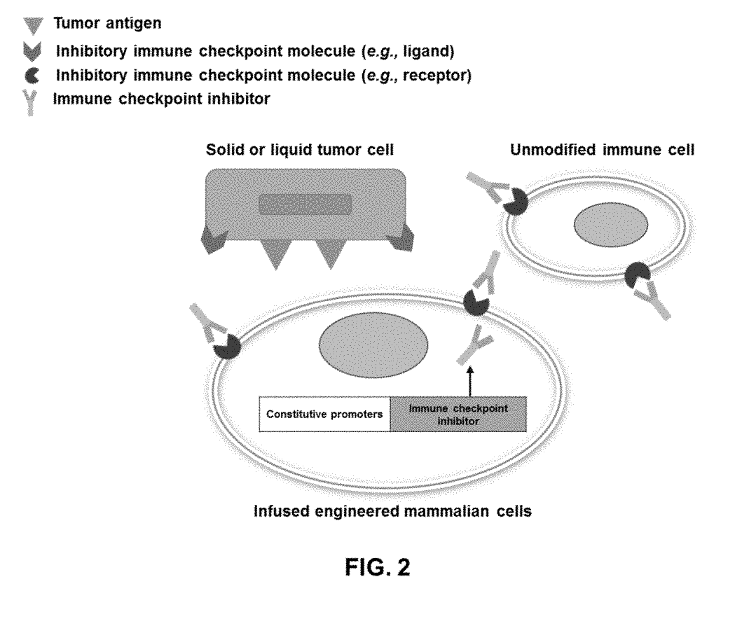

Constitutive promoters allow heterologous genes (also referred to as transgenes) to be expressed constitutively in the host cells. Exemplary constitutive promoters contemplated herein include, but are not limited to, Cytomegalovirus (CMV) promoters, human elongation factors-1alpha (hEF1α), ubiquitin C promoter (UbiC), phosphoglycerokinase promoter (PGK), simian virus 40 early promoter (SV40), and chicken β-Actin promoter coupled with CMV early enhancer (CAGG). The efficiencies of such constitutive promoters on driving transgene expression have been widely compared in a huge number of studies. For example, Michael C. Milone et al compared the efficiencies of CMV, hEF1α, UbiC and PGK to drive chimeric antigen receptor expression in primary human T cells, and concluded that hEF1α promoter not only induced the highest level of transgene expression, but was also optimally maintained in the CD4 and CD8 human T cells (Molecular Therapy, 17(8): 1453-1464 (2009)). In some embodiments, the promoter in the heterologous nucleic acid is a hEF1α promoter. An exemplary engineered mammalian cell comprising a heterologous nucleic acid encoding an immune checkpoint inhibitor operably linked to a constitutive promoter, wherein the immune checkpoint inhibitor blocks an inhibitory immune checkpoint molecule expressed on the tumor cells, is shown in FIG. 1. An exemplary engineered mammalian cell comprising a heterologous nucleic acid encoding an immune checkpoint inhibitor operably linked to a constitutive promoter, wherein the immune checkpoint inhibitor blocks an inhibitory immune checkpoint molecule expressed on the engineered mammalian cell and unmodified immune cells, is shown in FIG. 2.

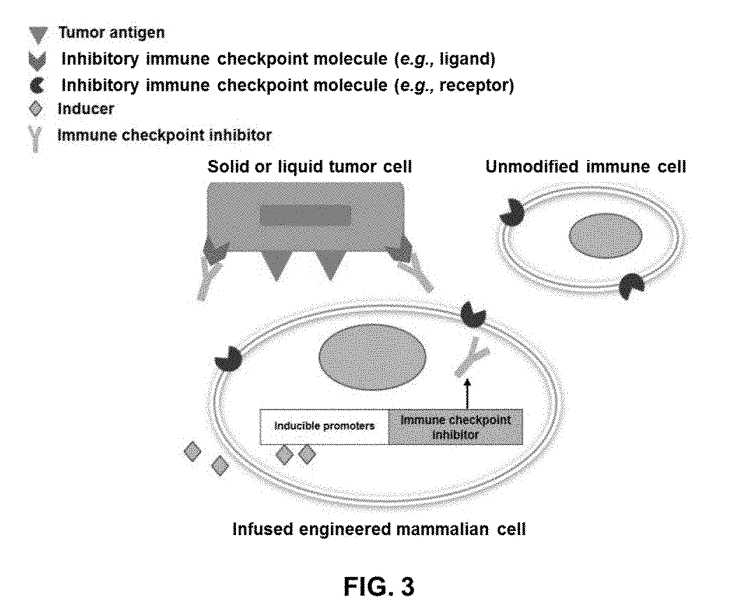

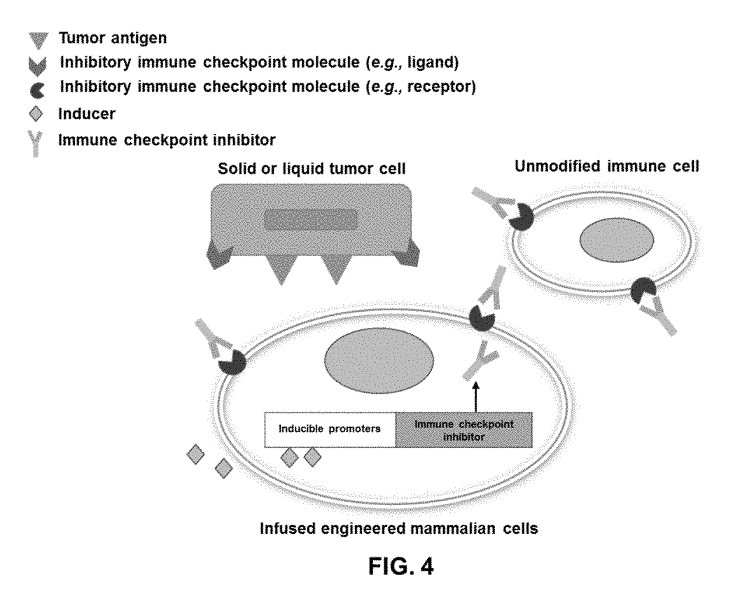

In some embodiments, the promoter is an inducible promoter. Inducible promoters belong to the category of regulated promoters. The inducible promoter can be induced by one or more conditions, such as a physical condition, microenvironment of the engineered mammalian cell, or the physiological state of the engineered mammalian cell, an inducer (i.e., an inducing agent), or a combination thereof. In some embodiments, the inducing condition does not induce the expression of endogenous genes in the engineered mammalian cell, and/or in the subject that receives the pharmaceutical composition. In some embodiments, the inducing condition is selected from the group consisting o£ inducer, irradiation (such as ionizing radiation, light), temperature (such as heat), redox state, tumor environment, and the activation state of the engineered mammalian cell. An exemplary engineered mammalian cell comprising a heterologous nucleic acid encoding an immune checkpoint inhibitor operably linked to an inducible promoter, wherein the immune checkpoint inhibitor blocks an inhibitory immune checkpoint molecule expressed on the tumor cells, is shown in FIG. 3. An exemplary engineered mammalian cell comprising a heterologous nucleic acid encoding an immune checkpoint inhibitor operably linked to an inducible promoter, wherein the immune checkpoint inhibitor blocks an inhibitory immune checkpoint molecule expressed on the engineered mammalian cell and unmodified immune cells, is shown in FIG. 4.

Thus, in some embodiments, there is provided a pharmaceutical composition comprising: a) an engineered mammalian (such as human) cell comprising a heterologous nucleic acid encoding an immunomodulator, wherein the heterologous nucleic acid is operably linked to an inducible promoter; and b) a pharmaceutically acceptable excipient. In some embodiments, the engineered mammalian cell is an immune cell (such as a PBMC, an NK cell, or a T cell). In some embodiments, the engineered mammalian cell is a stem cell. In some embodiments, the promoter is inducible by an inducing condition selected from inducer (such as small molecule, for example, tetracycline, or doxycycline), irradiation, temperature, redox state, tumor environment, and the activation state of the engineered mammalian cell. In some embodiments, the immunomodulator is an immune checkpoint inhibitor such as an inhibitor of CTLA-4, or an inhibitor of PD-1). In some embodiments, the immunomodulator is an immunoactivator. In some embodiments, the immunomodulator is a secreted protein. In some embodiments, the immunomodulator is an antibody (such as full-length antibody, scFv, singe-domain antibody, heavy chain-only antibody, or Fab). In some embodiments, the engineered mammalian cell further expresses on its surface a targeting molecule recognizing a tumor antigen. In some embodiments, the engineered mammalian cell further comprises a second heterologous nucleic acid encoding a therapeutic protein (such as a second immuno modulator, or a therapeutic protein that is not an immunomodulator, for example, chemotherapeutic antibody).

In some embodiments, the promoter is inducible by an inducer. In some embodiments, the inducer is a small molecule, such as a chemical compound. In some embodiments, the small molecule is selected from the group consisting of doxycycline, tetracycline, alcohol, metal, or steroids. Chemically-induced promoters have been most widely explored. Such promoters includes promoters whose transcriptional activity is regulated by the presence or absence of a small molecule chemical, such as doxycycline, tetracycline, alcohol, steroids, meal and other compounds. Doxycycline-inducible system with reverse tetracycline-controlled transactivator (rtTA) and tetracycline-responsive element promoter (TRE) is the most mature system at present. WO9429442 describes the tight control of gene expression in eukaryotic cells by tetracycline responsive promoters. WO9601313 discloses tetracycline-regulated transcriptional modulators. Additionally, Tet technology, such as the T et-on system, has described, for example, on the website of TetSystems.com. Any of the known chemically regulated promoters may be used to drive expression of the therapeutic protein in the present application.

In some embodiments, the inducer is a polypeptide, such as a growth factor, a hormone, or a ligand to a cell surface receptor, for example, a polypeptide that specifically binds a tumor antigen. In some embodiments, the polypeptide is expressed by the engineered mammalian cell. In some embodiments, the polypeptide is encoded by a nucleic add in the heterologous nucleic acid. Many polypeptide inducers are also known in the art, and they may be suitable for use in the present invention. For example, ecdysone receptor-based gene switches, progesterone receptor-based gene switches, and estrogen receptor based gene switches belong to gene switches employing steroid receptor derived transactivators (WO9637609 and WO9738117 etc.).

In some embodiments, the inducer comprises both a small molecule component and one or more polypeptides. For example, inducible promoters that dependent on dimerization of polypeptides are known in the art, and may be suitable for use in the present invention. The first small molecule CID system, developed in 1993, used FK1012, a derivative of the drug FK506, to induce homo-dimerization of FKBP. By employing similar strategies, Wu et al successfully make the CAR-T cells titratable through an ON-switch manner by using Rapalog/FKPB-FRB* and Gibberelline/GID1-GAI dimerization dependent gene switch (C.-Y. Wu et al., Science 350, aab4077 (2015)). Other dimerization dependent switch systems include Coumermycin/GyrB-GyrB (Nature 383 (6596): 178-81), and HaXS/Snap-tag-HaloTag (Chemistry and Biology 20 (4): 549-57).

In some embodiments, the promoter is alight-inducible promoter, and the inducing condition is light. Light inducible promoters for regulating gene expression in mammalian cells are also well-known in the art (see, for example, Science 332, 1565-1568 (2011); Nat. Methods 9, 266-269 (2012); Nature 500: 472-476 (2013); Nature Neuroscience 18:1202-1212 (2015)). Such gene regulation systems can be roughly put into two categories based on their regulations of (1) DNA binding or (2) recruitment of a transcriptional activation domain to a DNA bound protein. For instance, synthetic mammalian blue light controlled transcription system based on melanopsin which, in response to blue light (480 nm), triggers an intracellular calcium increase that result in calcineurin-mediated mobilization of NEAT, were developed and tested in mammalian cells. More recently, Motta-Mena et al described a new inducible gene expression system developed from naturally occurring EL222 transcription factor that confers high-level, blue light-sensitive control of transcriptional initiation in human cell lines and zebrafish embryos (Nat. Chem Biol. 10(3):196-202 (2014)). Additionally, the red light induced interaction of photoreceptor phytochrome B (Phy B) and phytochrome-interacting factor 6 (PIF 6) of Arabidopsis thaliana was exploited for a red light triggered gene expression regulation. Furthermore, ultraviolet B (UVB)-inducible gene expression system were also developed and proven to be efficient in target gene transcription in mammalian cells (Chapter 25 of Gene and Cell Therapy: Therapeutic Mechanisms and Strategies, Fourth Edition CRC Press, Jan. 20, 2015). Any of the lift-inducible promoters described herein may be used to drive expression of the therapeutic protein in the present invention.

In some embodiments, the promoter is alight-inducible promoter that is induced by a combination of a light-inducible molecule, and light. For example, a light-cleavable photocaged group on a chemical inducer keeps the inducer inactive, unless the photocaged group is removed through irradiation or by other means. Such light-inducible molecules include small molecule compounds, oligonucleotides, and proteins. For example, caged ecdysone, caged IP TG for use with the lacoperon, caged toy ocamycin for ribozyme-mediated gene expression, caved do xycycline for use with the Tet-on system, and caged Rapalog for light mediated FKBP/FRB dimerization have been developed (see, for example, Curr Op in Chem Biol. 16(3-4): 292-299 (2012)).

In some embodiments, the promoter is a radiation-inducible promoter, and the inducing condition is radiation, such as ionizing radiation. Radiation inducible promoters are also known in the art to control transgene expression. Alteration of gene expression occurs upon irradiation of cells. For example a group of genes known as “immediate early genes” can react promptly upon ionizing radiation. Exemplary immediate early genes include, but are not limited to, Erg-1, p21/WAF-1, GADD45alpha, t-PA, c-Fos, c-Jun, NF-kappaB, and API. The immediate early genes comprise radiation responsive sequences in their promoter regions. Consensus sequences have been found in the Erg-1 promoter, and are referred to as serum response elements or known as CArG elements. Combinations of radiation induced promoters and transgenes have been intensively studied and proven to be efficient with therapeutic benefits. See, for example, Cancer Biol Ther. 6(7):1005-12 (2007) and Chapter 25 of Gene and Cell Therapy: Therapeutic Mechanisms and Strategies, Fourth Edition CRC Press, Jan. 20, 2015. Any of the immediate early gene promoters or any promoter comprising a serum response element or CArG elements may be useful as a radiation inducible promoter to drive the expression of the therapeutic protein of the present invention.

In some embodiments, the promoter is a heat inducible promoter, and the inducing condition is heat. Heat inducible promoters driving transgene expression have also been widely studied in the art. Heat shock or stress protein (HSP) including Hsp90, Hsp70, Hsp60, Hsp40, Hsp10 etc. plays important roles in protecting cells under heat or other physical and chemical stresses. Several heat inducible promoters including heat-shock protein (HSP) promoters and growth arrest and DNA damage (GADD) 153 promoters have been attempted in pre-clinical studies. The promoter of human hsp70B gene, which was first described in 1985 appeals to be one of the most highly-efficient heat inducible promoters. Huang et al reported that after introduction of hsp70B-EGFP, hsp70B-TNFalpha and hsp70B-IL12 coding sequences, tumor cells expressed extremely high trans gene expression upon heat treatment, while in the absence of heat treatment, the expression of transgenes were not detected. And tumor growth was delayed significantly in the IL12 transgene plus heat treated group of mice in vivo (Cancer Res. 60:3435 (2000)). Another group of scientists linked the HSV-tk suicide gene to hsp70B promoter and test the system in nude mice bearing mouse breast cancer. Mice whose tumor had been administered the hsp70B-HSVtk coding sequence and heat treated showed tumor regression and a significant survival rate as compared to no heat treatment controls (Hum. Gene Ther. 11:2453 (2000)). Additional heat inducible promoters known in the art can be found in, for example, Chapter 25 of Gene and Cell Therapy: Therapeutic Mechanisms and Strategies, Fourth Edition CRC Press, Jan. 20, 2015. Any of the heat-inducible promoters discussed herein may be used to drive the expression of the therapeutic protein of the present invention.

In some embodiments, the promoter is inducible by a redox state. Exemplary promoters that are inducible by redox state include inducible promoter and hypoxia inducible promoters. For instance, Post D E et al developed hypoxia inducible factor (HIF) responsive promoter which specifically and strongly induce transgene expression in HIF-active tumor cells (Gene Ther. 8: 1801-1807 (2001); Cancer Res. 67: 6872-6881 (2007)).

In some embodiments, the promoter is inducible by the physiological state, such as an endogenous activation signal, of the engineered mammalian cell. In some embodiments, wherein the engineered mammalian cell is a T cell, the promoter is a T cell activation-dependent promoter, which is inducible by the endogenous activation signal of the engineered T cell. In some embodiments, the engineered T cell is activated by an inducer, such as PMA, ionomycin, or phytohaemagglutinin. In some embodiments, the engineered T cell is activated by recognition of a tumor antigen on the tumor cells via an endogenous T cell receptor, or an engineered receptor (such as recombinant TCR, or CAR). In some embodiments, the engineered T cell is activated by blockade of an immune checkpoint, such as by the immunomodulator expressed by the engineered T cell or by a second engineered mammalian cell. In some embodiments, the T cell activation-dependent promoter is an IL-2 promoter. In some embodiments, the T cell activation-dependent promoter is an NFAT promoter. In some embodiments, the T cell activation-dependent promoter is a NFκB promoter.

Without being bound by any theory or hypothesis, IL-2 expression initiated by the gene transcription from IL-2 promoter is a major activity of T cell activation. Un-specific stimulation of human T cells by Phorbol 12-myristate 13-acetate (PMA), or ionomycin, or phytohaemagglutinin results in IL-2 secretion from stimulated T cells. IL-2 promoter was explored for activation-induced transgene expression in genetically engineered T-cells (Virology Journal 3:97 (2006)). We found that IL-2 promoter is efficient to initiate reporter gene expression in the presence of PMA/PHA-P activation in human T cell lines. T cell receptor stimulation initiates a cascade of intracellular reactions causing an increasing of cytosolic calcium concentrations and resulting in nuclear translation of both NFAT and NFκB. Members of Nuclear Factor of Activated T cells (NFAT) are Ca²⁺ dependent transcription factors mediating immune response in T lymphocytes. NFAT have been shown to be crucial for inducible interleukine-2 (IL-2) expression in activated T cells (Mol Cell Biol. 15(11):6299-310(1995); Nature Reviews Immunology 5:472-484 (2005)). We found that NFAT promoter is efficient to initiate reporter gene expression in the presence of PMA/PHA-P activation in human T cell lines. Other pathways including nuclear factor kappa B (NFκB) can also be employed to control transgene expression via T cell activation.

### CAR or TCR

Any of the engineered mammalian cells described above may further express a cell surface molecule. The cell surface molecule comprises an extracellular domain and a transmembrane domain. In some embodiments, the cell surface molecule further comprises an intracellular effector domain, such as a primary intracellular signaling do main and/or a co-stimulatory signaling domain. In some embodiments, the cell surface molecule is an endogenous molecule. In some embodiments, the cell surface molecule is a heterologous molecule. In some embodiments, the cell surface molecule is an engineered molecule. In some embodiments, the cell surface molecule is encoded by the heterologous nucleic acid of the engineered mammalian cell. In some embodiments, the cell surface molecule is encoded by a second heterologous nucleic acid operably linked to a promoter (such as a constitutive promoter or an inducible promoter). In some embodiments, the cell surface molecule is introduced to the engineered mammalian cell by inserting proteins into the cell membrane while passing cells through a microfluidic system, such as CELL SQUEEZE (see, for example, U.S. Patent Application Publication No. 20140287509). The cell surface molecule may enhance the function of the engineered mammalian cell, such as by targeting the engineered mammalian cell, by transducing signals, and/or by enhancing cytotoxicity of the engineered mammalian cell. In some embodiments, the engineered mammalian cell does not express a cell surface molecule, such as CAR or a TCR.

In some embodiments, the cell surface molecule targets the engineered mammalian cell to tumor cells. In some embodiments, the cell surface molecule is a ligand of a cell surface receptor of tumor cells. In some embodiments, the engineered mammalian cell expresses on its surface a targeting molecule recognizing a tumor antigen. In some embodiments, the targeting molecule comprises an antibody fragment (such as an scFv or a single-domain antibody) against a tumor antigen. Exemplary tumor antigens include CD19, BCMA, NY-ESO-1, VEGFR2, MAGE-A3, CD20, CD22, CD33, CD38, CEA, EGFR (such as EGFRvIII), GD2, HER2, IGF1R, mesothelin, PSMA, ROR1, WT1, and other tumor antigens with clinical significance. In some embodiments, the cell surface molecule targets the engineered mammalian cell to the microenvironment of tumor cells, such as immune cells recruited to the tumor cells.

In some embodiments, the cell surface molecule is a chimeric effector molecule. In some embodiments, the chimeric effector molecule comprises one or more specific binding domains that target at least one tumor antigen, and one or more intracellular effector domains, such as one or more primary intracellular si paling domains and/or co-stimulatory signaling domains. In some embodiments, the cell surface molecule is not a CAR or a TCR.

In some embodiments, the cell surface molecule is a chimeric antigen receptor (CAR). CARS of the present invention comprise an extracellular domain comprising at least one targeting domain that specifically binds at least one tumor antigen, a transmembrane (TM) domain, and an intracellular signaling domain. In some embodiments, the intracellular signaling domain generates a signal that promotes an immune effector function of the CAR containing cell, e.g., a CAR-T cell. “Immune effector function or immune effector response” refers to function or response, e.g., of an immune effector cell, that enhances or promotes an immune attack of a target cell. For example an immune effector function or response may refer to a property of a T or NK cell that promotes killing or the inhibition of growth or proliferation, of a target cell. Examples of immune effector function, e.g., in a CAR-T cell, include cytolytic activity (such as antibody-dependent cellular toxicity, or ADCC) and helper activity (such as the secretion of cytokines). In some embodiments, the CAR has an intracellular signaling domain with an abolished or attenuated immune effector function. In some embodiments, the CAR has an intracellular signaling domain having no more than about any of 90%, 80%, 70%, 60%, 50%, 40%, 30%, 20%, 10% or less of an immune effector function (such as cytolytic function against target cells) compared to a CAR having a full-length and wildtype and optionally one or more co-stimulatory signaling domains. In some embodiments, the CAR alone does not induce cytolysis of the taut cells. In some embodiments, the intracellular signaling domain generates a signal that promotes proliferation and/or survival of the CAR containing cell. In some embodiments, the CAR comprises one or more intracellular signaling domains selected from the signaling domains of CD-28, CD137, CD3, CD27, CD40, ICOS, GITR, and OX40. The signaling domain of a naturally occurring molecule can comp rise the entire intracellular (i.e., cytoplasmic) portion, or the entire native intracellular signaling domain, of the molecule, or a fragment or derivative thereof.

In some embodiments, the targeting domain of the CAR is an antibody or an antibody fragment, such as an scFv, a Fv, a Fab, a (Fab′)₂, a single-domain antibody (sdAb), a VII or VL domain, or a VᵢₜH domain. In some embodiments, the one or more targeting domains of the CAR specifically bind to a single tumor antigen. In some embodiments, the CAR is a bispecific or multispecific CAR with targeting domains that bind two or more tumor antigens. In some embodiments, the tumor antigen is selected from the group consisting of CD19, BCMA, NY-ESO-1, VEGFR2, MAGE-A3, CD20, CD22, CD33, CD38, CEA, EGFR (such as EGFRvIII), GD2, HER2, IGF1R, mesothelin, PSMA, ROR1, WT1, and other tumor antigens with clinical significance, and combinations thereof.

In some embodiments, the transmembrane domain of the CAR comprises a transmembrane domain chosen from the transmembrane domain of an alpha, beta or zeta chain of a T-cell receptor, CD-28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL-2R beta, IL-2R gamma, IL-7Ra, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, 1TGAX, CD11c, ITGB1, CD29, ITGB2, CD18, LEA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C. In some embodiments, the transmembrane domain of the CAR is a CD4, CD3, CD8α, or CD-28 transmembrane domain. In some embodiments, the transmembrane domain of the CAR comprises a transmembrane domain of CD8α.

In some embodiments, the targeting domain is connected to the transmembrane domain by a hinge region. In one embodiment, the hinge region comprises the hinge region of CD8α.

In some embodiments, the CAR comprises a signal peptide (SP), such as a CD8α SP.

In some embodiments, the intracellular signaling domain comprises a primary intracellular signaling domain. “Primary intracellular signaling domain” refers to cytoplasmic signaling sequence that acts in a stimulatory manner to induce immune effector functions. In some embodiments, the primary intracellular signaling domain contains a signaling motif known as immunoreceptor tyrosine-based activation motif, or ITAM. In some embodiments, the primary intracellular signaling domain comprises a functional signaling domain of a protein selected from the group consisting of CD3 zeta, CD3 gamma, CD3 delta, CD3 epsilon, common FcR gamma(FCER1G), FcR beta (Fc Epsilon Rib), CD79a, CD79b, Fcgamma RIIa, DAP10, and DAP12. In some embodiments, the primary intracellular signaling domain comprises a nonfunctional or attenuated signaling domain of a protein selected from the group consisting of CD3 zeta, CD3 gamma, CD3 delta, CD3 epsilon, common FcR gamma (FCER1G), FcR beta (Fc Epsilon Rib), CD79a, CD79b, Fcgamma RIIa, DAP10, and DAP12. The nonfunctional or attenuated signaling domain can be a mutant signaling domain having a point mutation, insertion or deletion that attenuates or abolishes one or more immune effector functions, such as cytolytic activity or helper activity, including antibody-dependent cellular toxicity (ADCC). In some embodiments, the CAR comprises a nonfunctional or attenuated CD3 zeta (i.e. CD3ζ or CD3z) signaling domain. In some embodiments, the intracellular signaling domain does not comprise a primary intracellular signaling domain. CARs having no primary intracellular signaling domain, or having a nonfunctional or attenuated primary intracellular signaling domain are referred herein as “truncated CARs.” An attenuated primary intracellular signaling domain may induce no more than about any of 90%, 80%, 70%, 60%, 50%, 40%, 30%, 20%, 10% or less of an immune effector function (such as cytolytic function against target cells) compared to CARs having the same construct, but with the wildtype primary intracellular signaling domain. Engineered cells expressing truncated CARs alone may be unable to induce cytolysis of the target cells. Engineered cells with truncated CARS may have reduced toxicity and side effects, such as on-target off-cancer toxicity.

In some embodiments, the intracellular signaling domain comprises one or more (such as any of 1, 2, 3, or more) co-stimulatory signaling domains. “Co-stimulatory signaling domain” can be the intracellular portion of a co-stimulatory molecule. The term “co-stimulatory molecule” refers to a cognate binding partner on an immune cell (such as T cell) that specifically binds with a co-stimulatory ligand, thereby mediating a co-stimulatory response by the immune cell, such as, but not limited to, proliferation and survival. Co-stimulatory molecules are cell surface molecules other than antigen receptors or their ligands that contribute to an efficient immune response. A co-stimulatory molecule can be represented in the following protein families: TNF receptor proteins, Immunoglobulin-like proteins, cytokine receptors, integrins, signaling lymphocytic activation molecules (SLAM proteins), and activating NK cell receptors. Co-stimulatory molecules include, but are not limited to an MHC class 1 molecule, BTLA and a Toll ligand receptor, as well as OX40, CD27, CD-28, CDS, ICAM-1, LFA-1 (CD11a/CD18), ICOS (CD278), and 4-1BB (CD137). Further examples of such co-stimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD11d, ITGAE, CD103, ITGAL, CDIIa, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CDIOO (SEMA4D), CD69, SLAMF6 (NTB-A, Ly108), SLAM (SLAMF1, CD150, IPO-3), BLAME (SLAMF8), SELPLG (CD162), LTBR, LAT, GADS, SIP-76, PAG/Cbp, CD19a, and a ligand that specifically binds with CD83.

In some embodiments, the CAR comprises a single co-stimulatory signaling domain. In some embodiments, the CAR comprises two or more co-stimulatory signaling domains. In some embodiments, the intracellular signaling domain comprises a functional primary intracellular signaling domain and one or more co-stimulatory signaling domains. In some embodiments, the CAR is a truncated CAR. In some embodiments, the CAR does not comprise a functional primary intracellular signaling domain (such as CD3ζ). In some embodiments, the CAR comprises an intracellular signaling domain consisting of or consisting essentially of one or more co-stimulatory signaling domains. In some embodiments, the CAR comprises an intracellular signaling domain consisting of or consisting essentially of a nonfunctional or attenuated primary intracellular signaling domain (such as a mutant CD3ζ) and one or more co-stimulatory signaling domains. Upon binding of the targeting domain to tumor antigen, the co-stimulatory signaling domains of the CAR may transduce signals for enhanced proliferation, survival and differentiation of the engineered cells having the CAR (such as T cells), and inhibit activation induced cell death. In some embodiments, the co-stimulatory signaling domain comprises a functional signaling domain of a protein chosen from one or more of CD27, CD-28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, or NKG2D.

In some embodiments, the intracellular signaling domain comprises a functional signaling domain of CD137, such as the cytoplasmic domain of CD137. In some embodiments, the intracellular signaling domain comprises a functional primary signaling domain of CD3 zeta and a functional signaling domain of CD137. In some embodiments, the intracellular signaling domain comprises a nonfunctional or attenuated primary signaling domain of CD3 zeta and a functional signaling domain of CD137. In some embodiments, the intracellular signaling domain consists of or consists essentially of a functional signaling domain of CD137.

In some embodiments, the CAR comprises CD8α. SP, a targeting domain that specifically binds to a tumor antigen (such as EGFR, e.g., EGFRvIII, NY-ESO-1, or BCMA), CD8α hinge and transmembrane domain, a CD137 cytoplasmic domain, and CD3ζ In some embodiments, the CAR comprises CD8α, SP, a targeting domain that specifically binds to a tumor antigen (such as EGFR, e.g., EGFRvIII, NY-ESO-1, or BCMA), CD8α hinge and transmembrane domain, and a CD137 cytoplasmic domain.

Many chimeric antigen receptors are known in the art and may be suitable for the engineered mammalian cell of the present invention. CARs can also be constructed with a specificity for any cell surface marker by utilizing antigen binding fragments or antibody variable domains of, for example, antibody molecules. Any method for producing a CAR may be used herein. See, for example, U.S. Pat. No. 6,410,319, U.S. Pat. No. 7,446,191, U.S. Pat. No. 7,514,537, WO 2002/077029, WO2015/142675, US2010/065818, US 2010/025177, US 2007/059298, and Berger C. et al., J. Clinical Investigation 118: 1 294-308 (2008), which are hereby incorporated by reference. In some embodiments, the engineered mammalian immune cell is a CART cell.

Thus, in some embodiments, there is provided a pharmaceutical composition comprising: a) an engineered mammalian (such as human) immune cell comprising a heterologous nucleic acid encoding an immuno modulator, wherein the heterologous nucleic acid is operably linked to a promoter, wherein the engineered mammalian immune cell further expresses a CAR, and b) a pharmaceutically acceptable excipient. In some embodiments, the engineered mammalian immune cell is a PBMC, a T cell, or an NK cell. In some embodiments, the promoter is inducible, such as by the intracellular signaling domain of the CAR. In some embodiments, the immunomodulator is an immune checkpoint inhibitor (such as an inhibitor of CTLA-4, or an inhibitor of PD-1). In some embodiments, the immunomodulator is an immunoactivator. In some embodiments, the immunomodulator is a secreted protein. In some embodiments, the immunomodulator is an antibody (such as full-length antibody, scFv, singe-domain antibody, heavy chain-only antibody, or Fab). In some embodiments, the engineered mammalian cell further comprises a second heterologous nucleic acid encoding a therapeutic protein (such as a second immunomodulator, for example, an immuno activator; or a therapeutic protein that is net an immunomodulator, for example, chemotherapeutic antibody). In some embodiments, the immunoactivator is selected from the group consisting of IL-2, IL-7, IL-15, IL-21, IL-12, CCR4, CCR2b, Heparanase, CD137L, LEM, and Bcl-2. In some embodiments, the CAR is encoded by a third heterologous nucleic acid operably linked to a second promoter. In some embodiment, the second promoter is a constitutive promoter. In some embodiments, the second promoter is inducible, for example, by an inducing condition selected from inducer (such as small molecule, for example, tetracycline, or doxycycline), irradiation, temperature, redox state, tumor environment, and the activation state of the engineered mammalian immune cell. In some embodiments, the heterologous nucleic acid encoding the immunomodulator (such as the immune checkpoint inhibitor) and the heterologous nucleic acid encoding the CAR are operably linked to the same promoter, for example, a constitutive promoter, such as hEF1α. In some embodiments, the CAR targets a tumor antigen selected from the group consisting of CD19, BCMA, CD20, CD22, CD33, CD38, CEA, EGFR (such as EGFRvIII), GD2, HER2, IGF1R, mesothelin, PSMA, ROR1, WT1. In some embodiments, the CAR triggers cytolytic function, cytokine secretion, and/or proliferation of immune cells (including the engineered mammalian immune cell) upon binding of the engineered mammalian immune cell to tumor cells and upon secretion of the immunomodulator by the engineered mammalian immune cell. In some embodiments, the CAR comprises an intracellular signaling domain with an abolished or attenuated immune effector function. In some embodiments, the CAR is a truncated CAR. In some embodiments, the CAR does not comprise a primary intracellular signaling domain (such as CD3). In some embodiments, the CAR comprises a nonfunctional or attenuated primary intracellular signaling domain (such as a mutant CD3ζ). In some embodiments, the CAR alone does not induce cytolysis of the target cells.

In some embodiments, there is provided a pharmaceutical composition comprising: a) an engineered mammalian (such as human) CAR-T cell comprising a heterologous nucleic acid encoding an immunomodulator, wherein the heterologous nucleic acid is operably linked to a promoter; and b) a pharmaceutically acceptable excipient. In some embodiments, the promoter is inducible, such as by the intracellular signaling domain of the CAR. In some embodiments, the immunomodulator is an immune checkpoint inhibitor (such as an inhibitor of CTLA-4, or an inhibitor of PD-1). In some embodiments, the immunomodulator is an immunoactivator. In some embodiments, the immunomodulator is a secreted protein. In some embodiments, the immunomodulator is an antibody (such as full-length antibody, scFv, single-domain antibody, heavy chain-only antibody, or Fab). In some embodiments, the engineered mammalian cell further comprises a second heterologous nucleic acid encoding a therapeutic protein (such as a second immunomodulator, for example, an immuno activator; or a therapeutic protein that is not an immunomodulator, for example, chemotherapeutic antibody). In some embodiments, the immunoactivator is selected from the group consisting of IL-2, IL-7, IL-15, IL-21, IL-12, CCR4, CCR2b, Heparanase, CD137L, LEM, and Bcl-2. In some embodiments, the CAR is encoded by a third heterologous nucleic acid operably linked to a second promoter. In some embodiment, the second promoter is a constitutive promoter. In some embodiments, the second promoter is inducible, for example, by an inducing condition selected from inducer (such as small molecule, for example, tetracycline, or doxycycline), irradiation, temperature, redox state, tumor environment, and the activation state of the engineered mammalian CAR-T cell. In some embodiments, the heterologous nucleic acid encoding the immunomodulator (such as the immune checkpoint inhibitor) and the heterologous nucleic acid encoding the CAR are operably linked to the same promoter, for example, a constitutive promoter, such as hEF1α. In some embodiments, the CAR targets a tumor antigen selected from the group consisting of CD19, BCMA, NY-ESO-1, VEGFR2, MAGE-A3, CD20, CD22, CD33, CD38, CEA, EGFR (such as EGFRvIII), GD2, HER2, IGF1R, mesothelin, PSMA, ROR1, WT1. In some embodiments, the CAR triggers cytolytic function, cytokine secretion, and/or proliferation of immune cells (including the engineered mammalian CAR-T cell) upon binding of the engineered mammalian CAR-T cell to tumor cells and upon secretion of the immunomodulator by the engineered mammalian CAR-T cell. In some embodiments, the CAR comprises an intracellular signaling domain with an abolished or attenuated immune effector function. In some embodiments, the CAR is a truncated CAR. In some embodiments, the CAR does not comprise a primary intracellular signaling domain (such as CD3ζ). In some embodiments, the CAR comprises a nonfunctional or attenuated primary intracellular signaling domain (such as a mutant CD3ζ,). In some embodiments, the CAR alone does not induce cytolysis of the target cells.

In some embodiments, the cell surface molecule is a T cell receptor. In some embodiments, wherein the engineered mammalian cell is a T cell, the T cell receptor is an endogenous T cell receptor. In some embodiments, the engineered mammalian cell with the TCR is pre-selected. In some embodiments, the T cell receptor is a recombinant TCR. In some embodiments, the TCR is specific for a tumor antigen. In some embodiments, the tumor antigen is selected from the group consisting of CD19, BCMA, NY-ESO-1, VEGFR2, MAGE-A3, VEGFR2, MAGE-A3, CD20, CD22, CD33, CD38, CEA, EGFR (such as EGFRvIII), GD2, HER2, IGF1R, mesothelin, PSMA, ROR1, WT1, and other tumor antigens with clinical significance. In some embodiments, the tumor antigen is derived from an intracellular protein of tumor cells. Many TCRs specific for tumor antigens (including tumor-associated antigens) have been described, including, for example, NY-ESO-1 cancer-testis antigen, the p53 tumor suppressor antigens, TCRs for tumor antigens in melanoma MARTI, gp 100), leukemia (e.g., WT1, minor histocompatibility antigens), and breast cancer (HER2, NY-BR1, for example). Any of the TCRs known in the art may be used in the present application. In some embodiments, the TCR has an enhanced affinity to the tumor antigen. Exemplary TCRs and methods for introducing the TCRs to mammalian cells have been described, for example, in U.S. Pat. No. 5,830,755, and Kessels et al. Immunotherapy through TCR gene transfer. Nat. Immunol. 2, 957-961 (2001). In some embodiments, the engineered mammalian cell is a TCR-T cell.

Thus, in some embodiments, there is provided a pharmaceutical composition comprising: a) an engineered mammalian (such as human) immune cell comprising a heterologous nucleic acid encoding an immunomodulator, wherein the heterologous nucleic acid is operably linked to a promoter, wherein the engineered mammalian immune cell further expresses a TCR; and b) a pharmaceutically acceptable excipient. In some embodiments, the engineered mammalian immune cell is a PBMC or a T cell. In some embodiments, the promoter is inducible, such as by the intracellular signaling domain of the TCR In some embodiments, the immunomodulator is an immune checkpoint inhibitor (such as an inhibitor of CTLA-4, or an inhibitor of PD-1). In some embodiments, the immunomodulator is an immunoactivator. In some embodiments, the immunomodulator is a secreted protein. In some embodiments, the immunomodulator is an antibody (such as full-length antibody, scFv, singe-domain antibody, heavy chain-only antibody, or Fab). In some embodiments, the engineered mammalian immune cell further comprises a second heterologous nucleic acid encoding a therapeutic protein (such as a second immunomodulator, for example, an immunoactivator; or a therapeutic protein that is not an immunomodulator, for example, chemotherapeutic antibody). In some embodiments, the immunoactivator is selected from the group consisting of IL-2, IL-7, IL-15, IL-21, IL-12, CCR4, CCR2b, Heparanase, CD137L, LEM, and Bcl-2. In some embodiments, the TCR is encoded by a third heterologous nucleic acid operably linked to a second promoter. In some embodiment, the second promoter is a constitutive promoter. In some embodiments, the second promoter is inducible, for example, by an inducing condition selected from inducer (such as small molecule, for example, tetracycline, or doxycycline), irradiation, temperature, redox state, tumor environment, and the activation state of the engineered mammalian immune cell. In some embodiments, the TCR targets a tumor antigen selected from the group consisting of CD19, BCMA, NY-ESO-1, VEGFR2, MAGE-A3, CD20, CD22, CD33, CD38, CEA, EGFR (such as EGFRvIII), GD2, HER2, IGF1R, mesothelin, PSMA, ROR1, WT1. In some embodiments, the TCR triggers cytolytic function, cytokine secretion, and/or proliferation of immune cells (including the engineered mammalian immune cell) upon binding of the engineered mammalian immune cell to tumor cells and upon secretion of the immunomodulator by the engineered mammalian immune cell. In some embodiments, the TCR comprises an intracellular signaling domain with an abolished or attenuated immune effector function. In some embodiments, the CAR alone does not induce cytolysis of the target cells.

In some embodiments, there is provided a pharmaceutical composition comp rising: a) an engineered mammalian (such as human) TCR-T cell comp rising a heterologous nucleic acid encoding an immunomodulator, wherein the heterologous nucleic acid is operably linked to a promoter; and b) a pharmaceutically acceptable excipient. In some embodiments, the promoter is inducible, such as by the intracellular signaling domain of the TCR. In some embodiments, the immunomodulator is an immune checkpoint inhibitor (such as an inhibitor of CTLA-4, or an inhibitor of PD-1). In some embodiments, the immunomodulator is an immunoactivator. In some embodiments, the immunomodulator is a secreted protein. In some embodiments, the immunomodulator is an antibody (such as full-length antibody, scFv, single-domain antibody, heavy chain-only antibody, or Fab). In some embodiments, the engineered mammalian TCR-T cell further comprises a second heterologous nucleic acid encoding a therapeutic protein (such as a second immuno modulator, for example, an immunoactivator; or a therapeutic protein that is not an immunomodulator, for example, chemotherapeutic antibody). In some embodiments, the immunoactivator is selected from the group consisting of IL-2, IL-7, IL-21, IL-12, CCR4, CCR2b, Heparanase, CD137L, LEM, and Bcl-2. In some embodiments, the TCR is encoded by a third heterologous nucleic acid operably linked to a second promoter. In some embodiment, the second promoter is a constitutive promoter. In some embodiments, the second promoter is inducible, for example by an inducing condition selected from inducer (such as small molecule, for example, tetracycline, or doxycycline), irradiation, temperature, redox state, tumor environment, and the activation state of the engineered mammalian TCR-T cell. In some embodiments, the TCR targets a tumor antigen selected from the group consisting of CD19, BCMA, NY-ESO-1, VEGFR2, MAGE-A3, CD20, CD22, CD33, CD38, CEA, EGFR (such as EGFRvIII), GD2, HER2, IGF1R, mesothelin, PSMA, ROR1, WT1. In some embodiments, the TCR triggers cytolytic function, cytokine secretion, and/or proliferation of immune cells (including the engineered mammalian TCR-T cell) upon binding of the engineered mammalian TCR-T cell to tumor cells and upon secretion of the immunomodulator by the engineered mammalian TCR-T cell. In some embodiments, the TCR comprises an intracellular signaling domain with an abolished or attenuated immune effector function. In some embodiments, the TCR alone does not induce cytolysis of the target cells.

In some embodiments, the engineered mammalian cell further expresses both a CAR and a recombinant TCR.

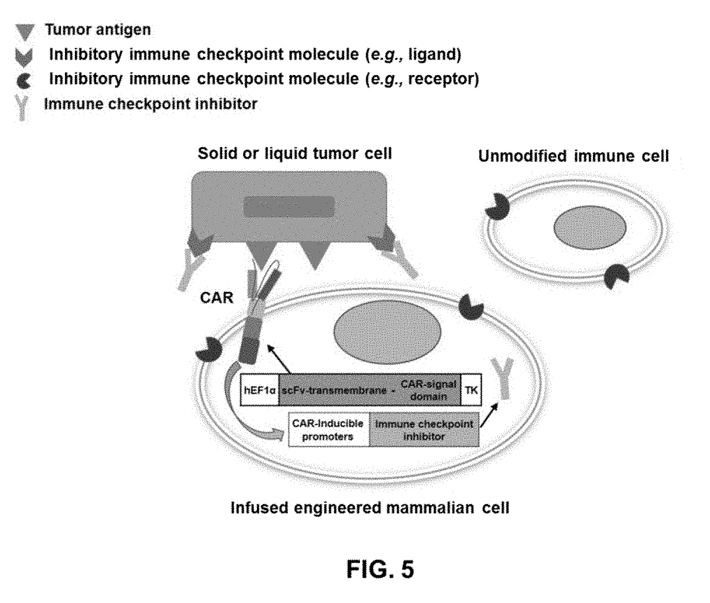

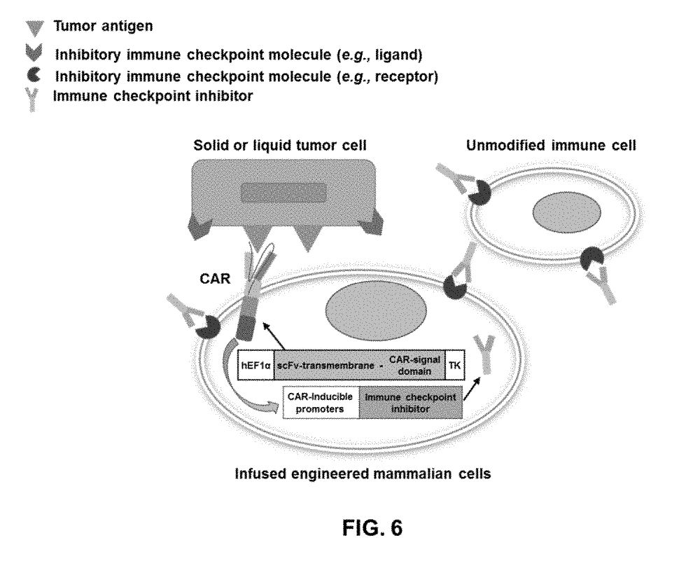

In some embodiments, the engineered mammalian cell further expresses a CAR or a TCR, and wherein the promoter for the immunomodulator is inducible by the intracellular signaling domain of the CAR or the TCR. In some embodiments, the promoter is a T cell activation-dependent promoter. For example, an engineered CAR-T or TCR-T cell of the present invention may transduce an activation signal via the intracellular signaling do main of the CAR or TCR upon binding to a tumor antigen on tumor cells. The activation signal may then induce the promoter operably linked to the nucleic acid encoding an immune checkpoint inhibitor, thereby increasing the secretion of the immune checkpoint inhibitor by the engineered CAR-T or TCR-T cell at the tumor site. Blockade of the immune checkpoint further activates the CAR-T and TCR-T cells, enhancing their cytotoxic activity against the tumor cells, while inducing proliferation of the CART and TCR-T cells, and stimulating the release of chemokines and cytokines, which further recruit endogenous T cells and other immune cells to the tumor site. Thereby, the CAR or TCR of the engineered T cell and the heterologous gene encoding the immune checkpoint inhibitor form a positive feedback loop that can enhance the local immune response at the tumor site. An exemplary engineered mammalian cell comprising a heterologous nucleic acid encoding an immune checkpoint inhibitor operably linked to a CAR-inducible promoter, and a second heterologous nucleic acid encoding a CAR operably linked to a constitutive promoter, wherein the immune checkpoint inhibitor blocks an inhibitory immune checkpoint molecule expressed on the tumor cells, is shown in FIG. 5. An exemplary engineered mammalian cell comprising a heterologous nucleic acid encoding an immune checkpoint inhibitor operably linked to a CAR-inducible promoter, and a second heterologous nucleic acid encoding a CAR operably linked to a constitutive promoter, wherein the immune checkpoint inhibitor blocks an inhibitory immune checkpoint molecule expressed on the engineered mammalian cell and unmodified immune cells, is shown in FIG. 6.

Thus, in some embodiments, there is provided a pharmaceutical composition comprising: a) an engineered mammalian (such as human) CAR-T cell comprising a heterologous nucleic acid encoding an immunomodulator, wherein the heterologous nucleic acid is operably linked to a T cell activation-dependent promoter; and b) a pharmaceutically acceptable excipient. In some embodiments, the T cell activation-dependent promoter is selected from an IL-2 promoter, an NFAT promoter, and an NFκB promoter. In some embodiments, the immunomodulator is an immune checkpoint inhibitor (such as an inhibitor of CTLA-4, or an inhibitor of PD-1). In some embodiments, the immunomodulator is an immunoactivator. In some embodiments, the immunomodulator is a secreted protein. In some embodiments, the immunomodulator is an antibody (such as full-length antibody, scFv, single-domain antibody, heavy chain-only antibody, or Fab). In some embodiments, the engineered mammalian CAR-T cell further comprises a second heterologous nucleic acid encoding a therapeutic protein (such as a second immunomodulator, for example, an immunoactivator; or a therapeutic protein that is not an immunomodulator, for example, chemotherapeutic antibody). In some embodiments, the immunoactivator is selected from the group consisting of IL-2, IL-7, IL-15, IL-21, IL-12, CCR4, CCR2b, Heparanase, CD137L, LEM, and Bcl-2. In some embodiments, the CAR is encoded by a third heterologous nucleic acid operably linked to a second promoter. In some embodiment, the second promoter is a constitutive promoter. In some embodiments, the second promoter is inducible, for example, by an inducing condition selected from inducer (such as small molecule, for example, tetracycline, or doxycycline), irradiation, temperature, redox state, tumor environment, and the activation state of the engineered mammalian CART cell. In some embodiments, the CAR targets a tumor antigen selected from the group consisting of CD19, BCMA, NY-ESO-1, VEGFR2, MAGE-A3, CD20, CD22, CD33, CD38, CEA, EGFR (such as EGFRvIII), GD2, HER2, IGF1R, mesothelin, PSMA, ROR1, WT1.

In some embodiments, there is provided a pharmaceutical composition comprising: a) an engineered mammalian (such as human) TCR-T cell comprising a heterologous nucleic acid encoding an immuno modulator, wherein the heterologous nucleic acid is operably linked to a T cell activation-dependent promoter; and b) a pharmaceutically acceptable excipient. In some embodiments, the T cell activation-dependent promoter is selected from an IL-2 promoter, an NFAT promoter, and an NFκB promoter. In some embodiments, the immunomodulator is an immune checkpoint inhibitor (such as an inhibitor of CTLA-4, or an inhibitor of PD-1). In some embodiments, the immunomodulator is an immunoactivator. In some embodiments, the immunomodulator is a secreted protein. In some embodiments, the immunomodulator is an antibody (such as full-length antibody, scFv, singe-domain antibody, heavy chain-only antibody, or Fab). In some embodiments, the engineered mammalian TCR-T cell further comprises a second heterologous nucleic acid encoding a therapeutic protein (such as a second immunomodulator, for example, an immunoactivator; or a therapeutic protein that is net an immunomodulator, for example, chemotherapeutic antibody). In some embodiments, the immunoactivator is selected from the group consisting of IL-2, IL-7, IL-15, IL-21, IL-12, CCR4, CCR2b, Heparanase, CD137L, LEM, and Bcl-2. In some embodiments, the TCR is encoded by a third heterologous nucleic acid operably linked to a second promoter. In some embodiment, the second promoter is a constitutive promoter. In some embodiments, the second promoter is inducible, for example, by an inducing condition selected from inducer (such as small molecule, for example, tetracycline, or doxycycline), irradiation, temperature, redox state, tumor environment, and the activation state of the engineered mammalian TCR-T cell. In some embodiments, the TCR targets a tumor antigen selected from the group consisting of CD19, BCMA, NY-ESO-1, VEGFR2, MAGE-A3, CD20, CD22, CD33, CD38, CEA, EGFR (such as EGFRvIII), GD2, HER2, IGF1R, mesothelin, PSMA, ROR1, WT1.

In some embodiments, the cell surface molecule (such as CAR and TCR) expressed by the engineered mammalian cell targets one or more tumor antigens. Tumor antigens are proteins that are produced by tumor cells that can elicit an immune response, particularly T-cell mediated immune responses. The selection of the targeted antigen of the invention will depend on the particular type of cancer to be treated. Exemplary tumor antigens include, for example, a glioma-associated antigen, carcinoembryonic antigen (CEA), β-human chorionic gonadotropin, alphafetoprotein (AFP), lectin-reactive AFP, thyroglobulin, RAGE-1, MN-CAIX, human telomerase reverse transcriptase, RU1, RU2 (AS), intestinal carboxyl esterase, mut hsp 70-2, M-CSF, prostase, prostate-specific antigen (PSA), PAP, NY-ESO-1, LAGE-1α, p53, prostein, PSMA, HER2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

In some embodiments, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and gp 100 in melanoma and prostatic acid phosphatase (PAP) and prostate-specific antigen (PSA) in prostate cancer. Other target molecules belong to the group of transformation-related molecules such as the oncogene HER2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immuno globulin antigen that is unique to the individual tumor. B-cell differentiation antigens such as CD19, CD20 and CD37 are other candidates for target antigens in B-cell lymphoma.

In some embodiments, the tumor antigen is a tumor-specific antigen (TSA) or a tumor-associated antigen (TAA). A TSA is unique to tumor cells and does not occur on other cells in the body. A TAA associated antigen is not unique to a tumor cell, and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development, when the immune system is immature, and unable to respond or they may be antigens that are normally present at extremely low levels on normal cells, but which are expressed at much higher levels on tumor cells.

Non-limiting examples of TSA or TAA antigens include the following Differentiation antigens such as MART-1/MelanA (MART-I), gp 100 (Pmel 17), tyrosinase, TRP-1, TRP-2 and tumor-specific multilineage antigens such as MAGE-1, MAGE-3, BAGE, GAGE-1, GAGE-2, pl5; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, pl85erbB2, pl80erbB-3, c-met, nm-23 HI, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alphafetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733 \Ep CAM, HTgp-175, M344, MA-50, MG7-Ag, MOV18, NB/70K, NY-CO-1, RCAS 1, SDCCAG16, TA-90\Mac-2 binding protein\cyclophilin C-associated protein, TAAL6, TAG72, TLP, and TPS.

In some embodiments, the tumor antigen targeted by the cell surface molecule (such as CAR or TCR) expressed by the engineered mammalian cell is EGFR.

Thus, in some embodiments, there is provided a pharmaceutical composition comprising: a) an engineered mammalian (such as human) CAR-T cell comprising a heterologous nucleic acid encoding an immuno modulator, wherein the heterologous nucleic acid is operably linked to a promoter, wherein the engineered CAR-T cell expresses a CAR targeting EGFR; and b) a pharmaceutically acceptable excipient. In some embodiments, the immunomodulator is an immune checkpoint inhibitor (such as an inhibitor of CTLA-4, or an inhibitor of PD-1). In some embodiments, the immunomodulator is an immunoactivator. In some embodiments, the immunomodulator is a secreted protein. In some embodiments, the immunomodulator is an antibody (such as full-length antibody, scFv, singe-domain antibody, heavy chain-only antibody, or Fab). In some embodiments, the engineered CAR-T cell further comprises a second heterologous nucleic acid encoding a therapeutic protein (such as a second immunomodulator, for example, an immunoactivator; or a therapeutic protein that is not an immunomodulator, for example, chemotherapeutic antibody). In some embodiments, the immunoactivator is selected from the group consisting of IL-2, IL-7, IL-15, IL-21, IL-12, CCR4, CCR2b, Heparanase, CD137L, LEM, and Bcl-2. In some embodiments, the CAR is encoded by a third heterologous nucleic acid operably linked to a second promoter. In some embodiment, the second promoter is a constitutive promoter. In some embodiments, the second promoter is inducible, for example, by an inducing condition selected from inducer (such as small molecule, for example, tetracycline, or doxycycline), irradiation, temperature, redox state, tumor environment, and the activation state of the engineered mammalian CAR-T cell. In some embodiments, the heterologous nucleic acid encoding the immuno modulator (such as the immune checkpoint inhibitor) and the heterologous nucleic acid encoding the CAR are operably linked to the same promoter, for example, a constitutive promoter, such as hEF1α. In some embodiments, the CAR triggers cytolytic function, cytokine secretion, and/or proliferation of T cells, including the engineered CAR-T cell, up on binding of the engineered mammalian CAR-T cell to tumor cells and upon secretion of the immunomodulator by the engineered CART cell. In some embodiments, the CAR comprises an intracellular signaling domain with an abolished or attenuated immune effector function. In some embodiments, the CAR is a truncated CAR In some embodiments, the CAR does not comp rise a primary intracellular signaling domain (such as CD3ζ). In some embodiments, the CAR comprises a nonfunctional or attenuated primary intracellular signaling domain (such as a mutant CD3ζ). In some embodiments, the CAR alone does not induce cytolysis of the target cells. In some embodiments, the pharmaceutical composition is useful for treating lung cancer, such as NSCLC.

In some embodiments, the tumor antigen targeted by the cell surface molecule (such as CAR or TCR) expressed by the engineered mammalian cell is EGFRvIII. EGFRvIII is a mutant form of the epidermal growth factor receptor, and is characterized by an 801 base pair in frame deletion of exons 2 to 7 near the amino terminal. In some embodiments, the engineered mammalian cell further expresses a CAR that targets EGFRvIII.

Glioblastoma (GBM) is the most common type of primary malignant brain tumor in adults, and remains to be one of the most lethal cancers. Even the patients are treated with multimodal therapies including surgical resection, chemotherapy and radiation, the median overall survival rate is no more than 15 months. Epidermal growth factor receptor variant III (EGFRvIII) is one of the most attractive tumor specific antigens on GBM. EGFRvIII is an in-frame deletion mutant of the wild type EGFR receptor. EGFRvIII is exclusively expressed on GBM cell surface and various types of cancers, but not on normal tissues and normal cells. CAR-T directing to EGFRvIII has shown great potential for GBM treatment, as reported by Miao H et al (2014).

Thus, in some embodiments, there is provided a pharmaceutical composition comprising: a) an engineered mammalian (such as human) CAR-T cell comprising a heterologous nucleic acid encoding an immuno modulator, wherein the heterologous nucleic acid is operably linked to a promoter, wherein the engineered CAR-T cell expresses a CAR targeting EGFRvIII; and b) a pharmaceutically acceptable excipient. In some embodiments, the immunomodulator is an immune checkpoint inhibitor (such as an inhibitor of CTLA-4, or an inhibitor of PD-1). In some embodiments, the immunomodulator is an immunoactivator. In some embodiments, the immunomodulator is a secreted protein. In some embodiments, the immunomodulator is an antibody (such as full-length antibody, scFv, single-domain antibody, heavy chain-only antibody, or Fab). In some embodiments, the engineered CART cell further comprises a second heterologous nucleic acid encoding a therapeutic protein (such as a second immunomodulator, for example, an immunoactivator; or a therapeutic protein that is not an immunomodulator, for example, chemotherapeutic antibody). In some embodiments, the immunoactivator is selected from the group consisting of IL-2, IL-7, IL-15, IL-21, IL-12, CCR4, CCR2b, Heparanase, CD137L, LEM, and Bcl-2. In some embodiments, the CAR is encoded by a third heterologous nucleic acid operably linked to a second promoter. In some embodiment, the second promoter is a constitutive promoter. In some embodiments, the second promoter is inducible, for example, by an inducing condition selected from inducer (such as small molecule, for example, tetracycline, or doxycycline), irradiation, temperature, redox state, tumor environment, and the activation state of the engineered mammalian CART cell. In some embodiments, the heterologous nucleic acid encoding the immunomodulator (such as the immune checkpoint inhibitor) and the heterologous nucleic acid encoding the CAR are operably linked to the same promoter, for example, a constitutive promoter, such as hEF1α. In some embodiments, the CAR triggers cytolytic function, cytokine secretion, and/or proliferation of T cells, including the engineered CAR-T cell, up on binding of the engineered mammalian CAR-T cell to tumor cells and upon secretion of the immunomodulator by the engineered CAR-T cell. In some embodiments, the CAR comprises an intracellular signaling domain with an abolished or attenuated immune effector function. In some embodiments, the CAR is a truncated CAR In some embodiments, the CAR does not comprise a primary intracellular signaling domain (such as CD3ζ). In some embodiments, the CAR comprises a nonfunctional or attenuated primary intracellular signaling domain (such as a mutant CD3ζ). In some embodiments, the CAR alone does not induce cytolysis of the target cells. In some embodiments, the pharmaceutical composition is useful for treating glioblastoma.

In some embodiments, the engineered mammalian cell (such as CAR-T) further expresses one or more (such as any of 1, 2, 3, or more) immunoactivators that promote T cell functions, such as T cell persistence and/or tissue homing A list of immunoactivators and their exemplary functions is shown in Table 1.

TABLE 1
Exemplary immunoactivators and their functions.

| Immuno- | |
|---|---|
| activator | Role in T cell functions |
| IL-2 | IL-2 can promote differentiation of certain immature T cells |
| | into regulatory T cells. IL-2 plays a crucial role in |
| | development and maintenance of Treg. IL-2 can also |
| | promote differentiation of T cells into effector T |
| | cells and into memory T cells when the initial T cell |
| | is stimulated by an antigen. |
| IL-7 | IL-7 can mediate homeostasis of naïve and memory CD4⁺, |
| | CD8⁺ T cells. IL-7 can also promote hematological |
| | malignancies (acute lymphoblastic leukemia, T cell |
| | lymphoma). |
| IL-15 | IL-15 is essential for maintenance of CD8⁺ Tmem, and can |
| | enhance NK cytotoxicity. IL-7 and IL-15 can act at each |
| | stage of the immune response to promote proliferation and |
| | survival of T cells. In this manner, a stable and protective, |
| | long-lived memory CD8⁺ T-cell pool can be propagated and |
| | maintained. |
| IL-21 | IL-21 can promote the maintenance of T(effs). |
| IL-12 | UCB-derived T cells cultured with IL-12 and IL-15 can |
| | generate >150-fold expansion with a unique central |
| | memory/effector phenotype. |
| CCR4 | CAR-T co-expressing CCR4 can have improved homing |
| | of anti-CD30 CAR-T. |
| CCR2b | Expression of chemokine receptor CCR2b can enhance |
| | tumor trafficking of GD2 chimeric antigen receptor |
| | T cells. |
| Heparanase | Heparanase promotes tumor infiltration and antitumor |
| | activity of CAR-redirected T-lymphocytes. |
| CD137L | Tumor necrosis factor ligand superfamily member 9 that |
| | supports longterm CD8⁺ T cell expansion. |
| LEM | LEM promotes CD8⁺ T cell immunity through effects on |
| | mitochondrial respiration. |
| Bcl-2 | T cells overexpressing Bcl-2 can be resistant to apoptosis. |

For example, numerous cytokines are reported to potentially affect T cell development, differentiation and homeostasis (Blood (2010) 115: 17). IL-2, IL-7, IL-15, and IL-21 are members of a cytokine family whose heteromeric receptors share the common γ chain (γ_{c}). Each cytokine has been described as a T-cell growth factor, and each has been used to augment the T-cell antitumor immune response, most notably IL-2. At a finer level, however, each cytokine possesses non-redundant functions that differentially shape T-cell responses: IL-2 plays a crucial role in the development and maintenance of regulatory T cells, a function not shared among other γc-cytokines. IL-7 mediates homeostasis of naive and memory CD4⁺ and CD8⁺ T cells. IL-15 is essential for maintenance of the CD8⁺ memory T-cell subset. The role of IL-21 in T cell mediated tumor immunity is less defined, with reports demonstrating its antitumor efficacy as a single agent, or in synergistic combination with IL-15. IL-15 and IL-21 may also promote long-term T-cell persistence through different mechanisms. There are also reports demonstrating that UCB-derived T cells cultured with IL-12 and IL-15 generated greater than 50-fold expansion with a unique central memory/effector phenotype (Leukemia (2015) 29: 415-422). In addition, LEM promotes CD8+ T cell immunity through effects on mitochondrial respiration (Science (2015) 348(6238): 995-1001), and Heparanase promotes tumor infiltration and antitumor activity of CAR-redirected T-lymphocytes (Nat. Med. (2015) 21(5): 524-529).

Tissue homing or T cell migration to the tumor site is also of great importance for adoptive T cell therapy, especially for solid tumors. At least 2 chemokine receptors have been reported to be able to enhance CAR-T cell trafficking to the tumor cells. John A Craddock et al reported CCR2b-expressing activated T cells (ATCs) are observed improved homing (>10-fold) to CCL2-secreting neuroblastoma compared to CCR2 negative ATCs (J. Immunoether. (2010) 33(8):780-788). Antonio Di Stasi et al reported T lymphocytes coexpressing CCR4 and a chimeric antigen receptor targeting CD30 have improved horning and antitumor activity in a Hodgkin tumor model (Blood (2009) 113(25)).

Activation-induced cell death (AICD) is a process of programmed cell death caused by the interaction of Fas receptors (Fas, CD95) and Fas ligands (FasL, CD95 ligand). AICD can be blocked by c-My c down-regulation and overexpression of CFLAR (caspase and FADD-like apoptosis regulator). Bcl-xL promotes in vitro lymphocyte survival under pro-apoptotic conditions (Gene Therapy (2002) 9: 527-535). In another report, Bcl-2 overexpression was found to enhance tumor-specific T-cell survival (Cancer Res (2005) 65(5):2001-2008).

Any one or more of the immunoactivators described herein may be further engineered to be co-expressed by the engineered mammalian cell on the same or different vectors as the chimeric effector molecule (such as CAR or TCR) to enhance the immune response triggered by binding of the engineered mammalian cell to the target cell. In some embodiments, the immune checkpoint inhibitor and/or the one or more immunoactivators, and the chimeric effector molecule (such as CAR or TCR) are encoded by different heterologous nucleic acids driven by different promoters. In some embodiments, the immune checkpoint inhibitor and/or the one or more immunoactivators, and the chimeric effector molecule (such as CAR or TCR) are encoded by a polycistronic nucleic acid driven by the same promoter. In some embodiments, the promoter is an inducible promoter. In some embodiments, the promoter is a constitutive promotor. In some embodiments, the promoter is hEF1α promoter. In some embodiments, there is provided a pharmaceutical composition comprising an engineered mammalian cell comprising a single vector encoding the immunomodulator(s), CAR (or TCR), and optionally other therapeutic protein(s). In some embodiments, there is provided a pharmaceutical composition comprising an engineered mammalian cell comprising a single heterologous nucleic acid that encodes the immunomodulator(s), CAR (or TCR), and optionally other therapeutic protein(s), wherein the single heterologous nucleic acid is operably linked to the same promoter. In some embodiments, use of a single vector or a single heterologous nucleic acid encoding the immunomodulator(s), CAR (or TCR), and optionally other therapeutic protein(s) in the pharmaceutical composition has several advantages, including, for example improved medicinal properties, homogeneity, and low cost.

Thus, in some embodiments, there is provided a pharmaceutical composition comprising: a) an engineered mammalian (such as human) CAR-T cell comprising a heterologous nucleic acid encoding an immune checkpoint inhibitor (such as a singe-domain antibody) and/or an immunoactivator, wherein the heterologous nucleic acid is operably linked to a promoter, and wherein the engineered CAR-T cell expresses a CAR; and b) a pharmaceutically acceptable excipient. In some embodiments, the heterologous nucleic acid encodes both the immune checkpoint inhibitor and the immunoactivator. In some embodiments, the heterologous nucleic acid encodes at least two immunoactivators. In some embodiments, the immune checkpoint inhibitor is an inhibitor of an immune checkpoint molecule selected from the group consisting of PD-1, PD-L1, PD-L2, CTLA-4, BLTA, TIM-3, or LAG-3. In some embodiments, the immune checkpoint inhibitor is an antibody (such as full-length antibody, scFv, single-domain antibody, heavy chain-only antibody, or Fab). In some embodiments, the immunoactivator is selected from the group consisting of IL-2, IL-7, IL-15, IL-21, IL-12, CCR4, CCR2b, Heparanase, CD137L, LEM, and Bcl-2. In some embodiments, the CAR targets a tumor antigen, such as EGFR, e.g., EGFRvIII, BCMA, or NY-ESO-1. In some embodiments, the engineered CAR-T cell further comprises a second heterologous nucleic acid encoding a therapeutic protein (such as a second immunomodulator, or a therapeutic protein that is not an immunomodulator, for example chemotherapeutic antibody). In some embodiments, the CAR is encoded by the heterologous nucleic acid operably linked to the promoter. In some embodiments, the CAR is encoded by a second heterologous nucleic acid operably linked to a second promoter. In some embodiment, the promoter and/or the second promoter are a constitutive promoter, such as hEF1α promoter. In some embodiments, the promoter and/or second promoter are inducible, for example, by an inducing condition selected from inducer (such as small molecule, for example, tetracycline, or doxycycline), irradiation, temperature, redox state, tumor environment, and the activation state of the engineered mammalian CAR-T cell. In some embodiments, the CAR triggers cytolytic function, cytokine secretion, and/or proliferation of T cells, including the engineered CAR-T cell, upon binding of the engineered mammalian CAR-T cell to tumor cells and upon secretion of the immunomodulator by the engineered CART cell. In some embodiments, the CAR comprises an intracellular signaling domain with an abolished or attenuated immune effector function. In some embodiments, the CAR is a truncated CAR. In some embodiments, the CAR does not comprise a primary intracellular signaling domain (such as CD3ζ). In some embodiments, the CAR comprises a nonfunctional or attenuated primary intracellular signaling domain (such as a mutant CD3ζ). In some embodiments, the CAR alone does not induce cytolysis of the target cells. In some embodiments, the CAR comprises CD8α. SP, a targeting domain that specifically binds to a tumor antigen (such as EGFR, e.g., EGFRvIII, BCMA, or NY-ESO-1), CD8α hinge and transmembrane domain, and a CD137 cytoplasmic domain.

In some embodiments, there is provided a pharmaceutical composition comprising: a) an engineered mammalian (such as human) immune cell comprising a vector encoding an immune checkpoint inhibitor and/or an immunoactivator, and a CAR; and b) a pharmaceutically acceptable excipient. In some embodiments, the vector encodes the immune checkpoint inhibitor, the immunoactivator, and the CAR. In some embodiments, the vector encodes at least two immunoactivators. In some embodiments, the engineered mammalian immune cell is a PBMC, a T cell, or an NK cell. In some embodiments, the vector comprises a first nucleic acid encoding the immune checkpoint inhibitor and a second nucleic acid en coding the CAR, wherein the first nucleic acid and the second nucleic acid are operably linked to the same promoter. In some embodiments, the vector comprises a first nucleic acid encoding the immune checkpoint inhibitor and a second nucleic acid encoding the CAR, wherein the first nucleic acid and the second nucleic acid are operably linked to different promoters. In some embodiments, the promoter is a constitutive promoter, such as hEF1α promoter. In some embodiments, the promoter is inducible. In some embodiments, the promoter is a T cell activation dependent promoter, such as an IL-2 promoter, an NFAT promoter, or an NFκB promoter. In some embodiments, the immune checkpoint inhibitor is an inhibitor of an immune checkpoint molecule selected from the group consisting of PD-1, PD-L1, PD-L2, CTLA-4, BLTA, TIM-3, or LAG-3. In some embodiments, the immune checkpoint inhibitor is an antibody (such as full-length antibody, scFv, single-domain antibody, heavy chain-only antibody, or Fab). In some embodiments, the immunoactivator is selected from the group consisting of IL-2, IL-7, IL-15, IL-21, IL-12, CCR4, CCR2b, Heparanase, CD137L, LENT, and Bcl-2. In some embodiments, the CAR targets a tumor antigen, such as EGFR, e.g., EGFRvIII, BCMA, or NY-ESO-1.

In some embodiments, there is provided a pharmaceutical composition comprising: a) an engineered mammalian (such as human) immune cell comprising a heterologous nucleic acid encoding an immune checkpoint inhibitor and/or an immunoactivator, and a CAR, wherein the heterologous nucleic acid is operably linked to a promoter; and b) a pharmaceutically acceptable excipient. In some embodiments, the heterologous nucleic acid encodes the immune checkpoint inhibitor, the immunoactivator, and the CAR. In some embodiments, the heterologous nucleic acid encodes at least two immunoactivators. In some embodiments, the engineered mammalian immune cell is a PBMC, a T cell, or an NK cell. In some embodiments, the promoter is a constitutive promoter, such as hEF1α promoter. In some embodiments, the promoter is inducible. In some embodiments, the promoter is a T cell activation dependent promoter, such as an IL-2 promoter, an NFAT promoter, or an NFκB promoter. In some embodiments, the immune checkpoint inhibitor is an inhibitor of an immune checkpoint molecule selected from the group consisting of PD-1, PD-L1, PD-L2, CTLA-4, BLTA, TIM-3, or LAG-3. In some embodiments, the immune checkpoint inhibitor is an antibody (such as full-length antibody, scFv, single-domain antibody, heavy chain-only antibody, or Fab). In some embodiments, the immunoactivator is selected from the group consisting of IL-2, IL-7, IL-15, IL-21, IL-12, CCR4, CCR2b, Heparanase, CD137L, LEM, and Bcl-2. In some embodiments, the CAR targets a tumor antigen, such as EGFR, e.g., EGFRvIII, BCMA, or NY-ESO-1.

In some embodiments, there is provided a pharmaceutical composition comprising: a) an engineered mammalian (such as human) immune cell comprising a heterologous nucleic acid encoding an immune checkpoint inhibitor and/or an immunoactivator, and a CAR comprising an intracellular signaling domain with an abolished or attenuated immune effector function, wherein the heterologous nucleic acid is operably linked to a promoter; and b) a pharmaceutically acceptable excipient. In some embodiments, the heterologous nucleic acid encodes the immune checkpoint inhibitor, the immunoactivator, and the CAR. In some embodiment, the heterologous nucleic acid encodes at least two immunoactivators. In some embodiments, the engineered mammalian immune cell is a PBMC, a T cell, or an NK cell. In some embodiments, the promoter is a constitutive promoter, such as hEF1α promoter. In some embodiments, the promoter is inducible. In some embodiments, the promoter is a T cell activation dependent promoter, such as an IL-2 promoter, an NFAT promoter, or an NFκB promoter. In some embodiments, the immune checkpoint inhibitor is an inhibitor of an immune checkpoint molecule selected from the group consisting of PD-1, PD-L1, PD-L2, CTLA-4, BLTA, TIM-3, or LAG-3. In some embodiments, the immune checkpoint inhibitor is an antibody (such as full-length antibody, scFv, single-domain antibody, heavy chain-only antibody, or Fab). In some embodiments, the immunoactivator is selected from the group consisting of IL-2, IL-7, IL-15, IL-21, IL-12, CCR4, CCR2b, Heparanase, CD137L, LEM, and Bcl-2. In some embodiments, the CAR targets a tumor antigen, such as EGFR, e.g., EGFRvIII, BCMA, or NY-ESO-1. In some embodiments, the CAR is a truncated CAR. In some embodiments, the CAR does not comprise a primary intracellular signaling domain (such as CD3ζ). In some embodiments, the CAR comprises a nonfunctional or attenuated primary intracellular signaling domain (such as a mutant CD3ζ). In some embodiments, the CAR alone does not induce cytolysis of the target cells. In some embodiments, the CAR comprises CD8α SP, a targeting domain that specifically binds to a tumor antigen (such as EGFR, e.g., EGFRvIII, NY-ESO-1, or BCMA), CD8α hinge and transmembrane domain, and a CD137 cytoplasmic domain.

### Mixture of Cells

In some embodiments, the pharmaceutical composition further comprises a second cell, wherein the second cell is a mammalian immune cell (such as T cell) that expresses a CAR or TCR. Any of the CARS or TCRs described in the above section may be expressed by the second cell, wherein the engineered mammalian cell only expresses the immunomodulator, and optional one or more additional therapeutic proteins (such as other immunomodulators or non-immunomodulators). At a tumor site, while the engineered mammalian cell of the pharmaceutical composition is capable of secreting the immunomodulator to block the inhibitory immune checkpoint or to activate the stimulatory immune checkpoint, the second mammalian immune cell expressing a CAR or TCR can be recruited to the tumor cells. The combined signal from the immunomodulator and the CAR or TCR allows activation of the second mammalian immune cell, and can trigger a strong immune response against the tumor cells. These two-component pharmaceutical compositions allow independent control (such as the timing, and amount) of secretion of the immunomodulator and additional therapeutic proteins by the engineered mammalian cell, and the activation of the second mammalian immune cell expressing the CAR or TCR. Precise control of the two types of cells may be useful in reducing undesirable side effects caused by either the immunomodulator or the CAR or TCR

Thus, in some embodiments, there is provided a pharmaceutical composition comprising: a) an engineered mammalian (such as human) cell comprising a heterologous nucleic acid encoding an immunomodulator, wherein the heterologous nucleic acid is operably linked to an inducible promoter; b) a second mammalian (such as human) immune cell expressing a CAR; and c) a pharmaceutically acceptable excipient. In some embodiments, the engineered mammalian cell is an immune cell (such as a PBMC, an NK cell, or a T cell). In some embodiments, the engineered mammalian cell is a stem cell. In some embodiments, the promoter is inducible by an inducing condition selected from inducer (such as small molecule, for example, tetracycline, or doxycycline), irradiation, temperature, redox state, tumor environment, and the activation state of the engineered mammalian cell. In some embodiments, the immunomodulator is an immune checkpoint inhibitor (such as an inhibitor of CTLA-4, or an inhibitor of PD-1). In some embodiments, the immunomodulator is an immunoactivator. In some embodiments, the immunomodulator is a secreted protein. In some embodiments, the immunomodulator is an antibody (such as full-length antibody, scFv, single-domain antibody, heavy chain-only antibody, or Fab). In some embodiments, the engineered mammalian cell further expresses on its surface a targeting molecule recognizing a tumor antigen. In some embodiments, the engineered mammalian cell further comprises a second heterologous nucleic acid encoding a therapeutic protein (such as a second immunomodulator, for example, an immunoactivator; or a therapeutic protein that is net an immunomodulator, for example, chemotherapeutic antibody). In some embodiments, the immunoactivator is selected from the group consisting of IL-2, IL-7, IL-15, IL-21, IL-12, CCR4, CCR2b, Heparanase, CD137L, LEM, and Bcl-2. In some embodiments, the second mammalian immune cell is a PBMC, a T cell or an NK cell. In some embodiments, the CAR targets a tumor antigen, such as EGFRvIII.

In some embodiments, there is provided a pharmaceutical composition comprising: a) an engineered mammalian (such as human) cell comprising a heterologous nucleic acid encoding an immunomodulator, wherein the heterologous nucleic acid is operably linked to an inducible promoter; b) a second mammalian (such as human) immune cell expressing a TCR; and c) a pharmaceutically acceptable excipient. In some embodiments, the engineered mammalian cell is an immune cell (such as a PBMC, an NK cell, or a T cell). In some embodiments, the engineered mammalian cell is a stem cell. In some embodiments, the promoter is inducible by an inducing condition selected from inducer (such as small molecule, for example, tetracycline, or doxycycline), irradiation, temperature, redox state, tumor environment, and the activation state of the engineered mammalian cell. In some embodiments, the immunomodulator is an immune checkpoint inhibitor (such as an inhibitor of CTLA-4, or an inhibitor of PD-1). In some embodiments, the immunomodulator is an immunoactivator. In some embodiments, the immunomodulator is a secreted protein. In some embodiments, the immunomodulator is an antibody (such as full-length antibody, scFv, single-domain antibody, heavy chain-only antibody, or Fab). In some embodiments, the engineered mammalian cell further expresses on its surface a targeting molecule recognizing a tumor antigen. In some embodiments, the engineered mammalian cell further comprises a second heterologous nucleic acid encoding a therapeutic protein (such as a second immunomodulator, for example, an immunoactivator; or a therapeutic protein that is net an immunomodulator, for example, chemotherapeutic antibody). In some embodiments, the immunoactivator is selected from the group consisting of IL-2, IL-7, IL-15, IL-21, IL-12, CCR4, CCR2b, Heparanase, CD137L, LEM, and Bcl-2. In some embodiments, the second mammalian immune cell is a T cell or a TCR-T. In some embodiments, the TCR targets a tumor antigen, such as EGFRvIII.

The second mammalian immune cell may be from the same or different source as the engineered mammalian cell. The second mammalian immune cell may also be of the same type (including subpopulation) or different type as the engineered mammalian cell. In some embodiments, both of the second mammalian immune cell and the engineered mammalian cellar e autologous. In some embodiments, both of the second mammalian immune cell and the engineered mammalian cell are allogenic. In some embodiments, both of the second mammalian immune cell and the engineered mammalian cell are obtained from the same individual. In some embodiments, both of the second mammalian immune cell and the engineered mammalian cell are obtained from different individuals. In some embodiments, the second mammalian immune cell is autologous, while the engineered mammalian cell is allogenic. In some embodiments, the engineered mammalian cell is autologous, while the second mammalian immune cell is allogenic.

The second mammalian immune cell and the engineered mammalian cell may be present in the pharmaceutical composition in any suitable ratio. In some embodiments, the ratio between the second mammalian immune cell and the engineered mammalian cell in the pharmaceutical composition is about any of 1:100, 1:50, 1:20, 1:10, 1:5, 1:2, 1:1, 2:1, 5:1, 10:1, 20:1, 100:1. In some embodiments, the ratio between the second mammalian immune cell and the engineered mammalian cell in the pharmaceutical composition is any of about 1:100 to about 1:50, about 1:50 to about 1:10, about 1:20 to about 1:10, about 1:10 to about 1:5, about 1:5 to about 1:2, about 1:2 to about 1:1, about 1:2 to about 2:1, about 1:1 to about 2:1, about 2:1 to about 5:1, about 5:1 to about 10:1, about 10:1 to about 20:1, about 10:1 to about 50:1, about 50:1 to about 100:1, about 1:10 to about 10:1, or about 1:100 to about 100:1.

### Excipient

The pharmaceutical compositions of the present invention are useful for therapeutic purposes. Thus, different from other compositions comprising engineered mammalian cells, such as production cells that express immunomodulators or other therapeutic proteins, the pharmaceutical compositions of the present invention comp rises a pharmaceutically acceptable excipient suitable for administration to an individual.

Suitable pharmaceutically acceptable excipient may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. In some embodiments, the pharmaceutically acceptable excipient comprises autologous serum. In some embodiments, the pharmaceutically acceptable excipient comprises human serum. In some embodiments, the pharmaceutically acceptable excipient is non-toxic, biocompatible, non-immunogenic, biodegradable, and can avoid recognition by the host's defense mechanism. The excipient may also contain adjuvants such as preserving stabilizing, wetting, emulsifying agents and the like. In some embodiments, the pharmaceutically acceptable excipient enhances the stability of the engineered mammalian cell or the immunomodulator or other therapeutic proteins secreted thereof. In some embodiments, the pharmaceutically acceptable excipient reduces aggregation of the immunomodulator or other therapeutic proteins secreted by the engineered mammalian cell. The final form may be sterile and may also be able to pass readily through an injection device such as a hollow needle. The proper viscosity may be achieved and maintained by the proper choice of excipients.

In some embodiments, the pharmaceutical composition is formulated to have a pH in the range of about 4.5 to about 9.0, including for example pH ranges of about any one of 5.0 to about 8.0, about 6.5 to about 7.5, or about 6.5 to about 7.0. In some embodiments, the pharmaceutical composition can also be made to be isotonic with blood by the addition of a suitable tonicity modifier, such as glycerol.

In some embodiments, the pharmaceutical composition is suitable for administration to a human. In some embodiments, the pharmaceutical composition is suitable for administration to a human by parenteral administration. Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizing agents, and preservatives. The formulations can be presented in unit-dose or multi-dose sealed containers, such as ampules and vials, and can be stored in a condition requiring only the addition of the sterile liquid excipient methods of treatment, methods of administration, and dosage regimens described herein (i.e., water) for injection, immediately prior to use. In some embodiments, the pharmaceutical composition is contained in a single-use vial, such as a single-use sealed vial. In some embodiments, the pharmaceutical composition is contained in a multi-use vial. In some embodiments, the pharmaceutical composition is contained in bulk in a container. In some embodiments, the pharmaceutical composition is cryopreserved.

In some embodiments, the pharmaceutical composition is formulated for intravenous administration. In some embodiments, the pharmaceutical composition is formulated for subcutaneous administration. In some embodiments, the pharmaceutical composition is formulated for local administration to a tumor site. In some embodiments, the pharmaceutical composition is formulated for intratumoral injection.

In some embodiments, the pharmaceutical composition must meet certain standards for administration to an individual. For example, the United States Food and Drug Administration has issued regulatory guidelines setting standards for cell-based immunotherapeutic products, including 21 CFR 610 and 21 CFR 610.13. Methods are known in the art to assess the appearance, identity, purity, safety, and/or potency of pharmaceutical compositions. In some embodiments, the pharmaceutical composition is substantially free of extraneous protein capable of producing allergenic effects, such as proteins of an animal source used in cell culture other than the engineered mammalian immune cells. In some embodiments, “substantially free” is less than about any of 10%, 5%, 1%, 0.1%, 0.01%, 0.001%, 1 ppm or less of total volume or weight of the pharmaceutical composition. In some embodiments, the pharmaceutical composition is prepared in a GMP-level workshop. In some embodiments, the pharmaceutical composition comprises less than about 5 EU/kg body weight/hr of endotoxin for parenteral administration. In some embodiments, at least about 70% of the engineered mammalian cells in the pharmaceutical composition are alive for intravenous administration. In some embodiments, the pharmaceutical composition has a “no growth” result when assessed using a 14-day direct inoculation test method as described in the United States Pharmacopoeia (USP). In some embodiments, prior to administration of the pharmaceutical composition, a sample including both the engineered mammalian cells and the pharmaceutically acceptable excipient should betaken for sterility testing approximately about 48-72 hours prior to the final harvest (or coincident with the last re-feeding of the culture). In some embodiments, the pharmaceutical composition is free of mycoplasma contamination. In some embodiments, the pharmaceutical composition is free of detectable microbial agents. In some embodiments, the pharmaceutical composition is free of communicable disease agents, such as HIV type I, HIV type II, HBV, HCV, Human T-lymphotropic virus, type I; and Human T-lymphotropic virus, type II.

### III. Methods of Preparation

Further provided are methods of preparing any of the pharmaceutical compositions described herein, comprising introducing into a mammalian cell a vector comprising the heterologous nucleic acid.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. In general, a suitable vector contains an origin of replication functional in at least one organism, a promoter sequence, convenient restriction endonuclease sites, and one or more selectable markers. The term “vector” should also be construed to include non-plasmid and non-viral compounds which facilitate transfer of nucleic acid into cells, such as, for example, poly sine compounds, liposomes, and the like.

In some embodiments, the vector is a viral vector. Examples of viral vectors include, but are not limited to, adenoviral vectors, adeno-associated virus vectors, lentiviral vector, retroviral vectors, vaccinia vector, herpes simplex viral vector, and derivatives thereof. Viral vector technology is well known in the art and is described, for example, in Sambrook et al. (2001, Molecular Cloning: A Laboratory Manual, Cold Spring Harbor Laboratory, New York), and in other virology and molecular biology manuals.

A number of viral based systems have been developed for gene transfer into mammalian cells. For example, retroviruses provide a convenient platform for gene delivery systems. The heterologous nucleic acid am be inserted into a vector and packaged in retroviral particles using techniques known in the art. The recombinant virus can then be isolated and delivered to the engineered mammalian cell in vitro or ex vivo. A number of retroviral systems are known in the art. In some embodiments, adenovirus vectors are used. A number of adenovirus vectors are known in the art. In some embodiments, lentivirus vectors are used. In some embodiments, self-inactivating lentiviral vectors are used. For example, self-inactivating lentiviral vectors carrying the immunomodulator (such as immune checkpoint inhibitor) coding sequence and/or self-inactivating lentiviral vectors carrying chimeric antigen receptors can be packaged with protocols known in the art. The resulting lentiviral vectors can be used to transduce a mammalian cell (such as primary human T cells) using methods known in the art.

The host cells can be prepared using a variety of methods known in the art. For example, primary immune cells, such as T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, immune cells (such as T cells) can be obtained from a unit of blood collected from an individual using any number of techniques known in the art, such as FICOLL™ separation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to p lace the cells in an appropriate buffer or media for subsequent processing steps. In some embodiments, the cells are washed with phosphate buffered saline (PBS), or a wash solution lacking divalent cations, such as calcium and magnesium. As those of ordinary skill in the art would readily appreciate a washing step may be accomplished by methods known to those in the art, such as by using a semi-automated “flow-through” centrifuge (for example, the Cobe2991 cell processor, the Baxter CytoMate, or the Haemonetics Cell Saver 5) according to the manufacturer's instructions. After washing the cells may be resuspended in a variety of biocompatible buffers, such as, for Example, Ca²⁺-free, Mg²⁺-free PBS, PlasmaLyte A, or other saline solution with or without buffer. Alternatively, the undesirable components of the apheresis sample may be removed and the cells directly resuspended in culture media.

In some embodiments, primary T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3′, CD-28⁺, CD4⁺, CD8⁺, CD45RA, and CD45RO cells, can be further isolated by positive or negative selection techniques. For example, in one embodiment, T cells are isolated by incubation with anti-CD3/anti-CD-28 (i.e., 3×28)-conjugated beads, such as DYNABEADS® M-450 CD3/CD-28 T, for a time period sufficient for positive selection of the desired T cells.

In some embodiments, a T cell population may further be enriched by negative selection using a combination of antibodies directed to surface markers unique to the negatively selected cells. For example, one method involves cell sorting and/or selection via negative magnetic immunoadherence or flow cytometry that uses a cocktail of monoclonal antibodies directed to cell surface markers present on the cells negatively selected. For example, to enrich for CD4 cells by negative selection, a monoclonal antibody cocktail typically includes antibodies to CD14, CD20, CD11b, CD16, HLA-DR, and CD8. In certain embodiments, it may be desirable to enrich for or positively select for regulatory T cells which typically express CD4⁺, CD25⁺, CD62L^{hi}, GITR⁺, and FoxP3⁺. Alternatively, in certain embodiments, T regulatory cells are depleted by anti-CD25 conjugated beads or other similar methods of selection.

Methods of introducing vectors into a mammalian cell are known in the art. The vectors can be transferred into a host cell by physical, chemical, or biological methods.

Physical methods for introducing the vector into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001) Molecular Cloning A Laboratory Manual, Cold Spring Harbor Laboratory, New York. In some embodiments, the vector is introduced into the cell by electroporation.

Biological methods for introducing the heterologous nucleic acid into a host cell include the use of DNA and RNA vectors. Viral vectors have become the most widely used method for inserting genes into mammalian, e.g., human cells.

Chemical means for introducing the vector into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro is a liposome (e.g., an artificial membrane vesicle).

In some embodiments, the transduced or transfected mammalian cell is propagated ex vivo after introduction of the heterologous nucleic acid. In some embodiments, the transduced or transfected mammalian cell is cultured to prop agate for at least about any of 1 day, 2 days, 3 days, 4 days, 5 days, 6 days, 7 days, 10 days, 12 days, or 14 days. In some embodiments, the transduced or transfected mammalian cell is cultured for no more than about any of 1 day, 2 days, 3 days, 4 days, 5 days, 6 days, 7 days, 10 days, 12 days, or 14 days. In some embodiments, the transduced or transfected mammalian cell is further evaluated or screened to select the engineered mammalian cell.

Reporter genes may be used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et at FEBS Letters 479: 79-82 (2000)). Suitable expression systems are well known and may be prepared using known techniques or obtained commercially.

Other methods to confirm the presence of the heterologous nucleic acid in the mammalian cell, include, for example, molecular biological assays well known to those of skill in the art, such as Southern and Northern blotting, RT-PCR and PCR; biochemical assays, such as detecting the presence or absence of a particular peptide, e.g., by immunological methods (such as ELISAs and Western blots).

For example, the secretion of immuno modulators (such as immune checkpoint inhibitors) in the culture of transduced mammalian cells (such as primary T cells) can be detected by enzyme-linked immunosorbent assay (ELISA) or by flow cytometry. Furthermore, biological functions of the secreted immunomodulators (such as immune checkpoint inhibitors) can be assayed in vitro using reporter assay or cytokine release assays. Such reporter assays can be performed on in-house developed stable reporter tumor cells. In the cases of engineered T cells, cytokine release assays can be performed to detect T cell restoration level in response to secretion of immune checkpoint inhibitors by the engineered T cell. In the cases of engineered CAR-T cells, the capability of secretion of immuno modulators (such as immune checkpoint inhibitors) on enhancing CAR-T cytotoxicity on tumor cells can be assayed with in vitro co-culture assays, in which T cells are co-cultured with tumor cells at several ratios for a period of time.

### IV. Methods of Treating Cancer

One aspect of the present app heat ion relates to methods of treating cancer using any of the pharmaceutical compositions described above.

In some embodiments, there is provided a method of treating a cancer in an individual (such as a human individual), comprising administering to the individual an effective amount of a pharmaceutical composition comprising a) an engineered mammalian cell comprising a heterologous nucleic acid encoding an immunomodulator, wherein the heterologous nucleic acid is operably linked to a promoter; and b) a pharmaceutically acceptable excipient. In some embodiments, the engineered mammalian cell is an immune cell (such as a PBMC, an NK cell, or a T cell). In some embodiments, the engineered mammalian cell is a stem cell. In some embodiments, the promoter is inducible by an inducing condition selected from inducer (such as small molecule, for example tetracycline, or doxycycline), irradiation, temperature, redox state, tumor environment, and the activation state of the engineered mammalian cell. In some embodiments, the immunomodulator is an immune checkpoint inhibitor (such as an inhibitor of CTLA-4, or an inhibitor of PD-1). In some embodiments, the immunomodulator is an immunoactivator. In some embodiments, the immunomodulator is a secreted protein. In some embodiments, the immunomodulator is an antibody (such as full-length antibody, scFv, single-domain antibody, heavy chain-only antibody, or Fab). In some embodiments, the engineered mammalian cell further expresses on its surface a targeting molecule recognizing a tumor antigen (such as EGFR, e.g., EGFRvIII, BCMA, or NY-ESO-1). In some embodiments, the engineered mammalian cell does not express a CAR or a TCR. In some embodiments, the engineered mammalian cell further comprises a second heterologous nucleic acid encoding a therapeutic protein (such as a second immunomodulator, for example, an immuno activator; or a therapeutic protein that is not an immunomodulator, for example, chemotherapeutic antibody). In some embodiments, the immunoactivator is selected from the group consisting of IL-2, IL-7, IL-15, IL-21, IL-12, CCR4, CCR2b, Heparanase, CD137L, LEM, and Bcl-2. In some embodiments, the engineered mammalian cell is obtained from the individual. In some embodiments, the engineered mammalian cell is allogenic to the individual.

In some embodiments, there is provided a method of treating a cancer in an individual (such as a human individual), comprising administering to the individual an effective amount of a pharmaceutical composition comprising: a) an engineered mammalian (such as human) immune cell comprising a heterologous nucleic acid encoding an immuno modulator, wherein the heterologous nucleic acid is operably linked to a promoter, wherein the engineered mammalian immune cell further expresses a CAR or TCR; and b) a pharmaceutically acceptable excipient. In some embodiments, the engineered mammalian immune cell is a PBMC, a T cell, or an NK cell. In some embodiments, the promoter is inducible, such as by the intracellular signaling domain of the CAR or TCR In some embodiments, the promoter is a T cell activation dependent promoter, such as an IL-2 promoter, an NFAT promoter, or an NFκB promoter. In some embodiments, the immunomodulator is an immune checkpoint inhibitor (such as an inhibitor of CTLA-4, or an inhibitor of PD-1). In some embodiments, the immunomodulator is an immunoactivator. In some embodiments, the immunomodulator is a secreted protein. In some embodiments, the immunomodulator is an antibody (such as full-length antibody, scFv, single-domain antibody, heavy chain-only antibody, or Fab). In some embodiments, the engineered mammalian cell further comprises a second heterologous nucleic acid encoding a therapeutic protein (such as a second immunomodulator, for example, an immunoactivator; or a therapeutic protein that is net an immunomodulator, for example, chemotherapeutic antibody, e.g., an anti-HER2 antibody). In some embodiments, the immunoactivator is selected from the group consisting of IL-2, IL-7, IL-15, IL-21, IL-12, CCR4, CCR2b, Heparanase, CD137L, LEM, and Bcl-2. In some embodiments, the CAR is encoded by a third heterologous nucleic acid operably linked to a second promoter. In some embodiment, the second promoter is a constitutive promoter. In some embodiments, the second promoter is inducible, for example, by an inducing condition selected from inducer (such as small molecule, for example, tetracycline, or doxycycline), irradiation, temperature, redox state, tumor environment, and the activation state of the engineered mammalian immune cell. In some embodiments, the CAR or TCR targets a tumor antigen, such as EGFR, e.g., EGFRvIII, BCMA, or NY-ESO-1. In some embodiments, the engineered mammalian immune cell is obtained from the individual. In some embodiments, the engineered mammalian immune cell is allogenic to the individual.

In some embodiments, there is provided a method of treating a cancer in an individual (such as a human individual), comprising administering to the individual an effective amount of a pharmaceutical composition comprising a) an engineered mammalian (such as human) immune cell comprising a heterologous nucleic acid encoding an immune checkpoint inhibitor and/or an immunoactivator, and a CAR, wherein the heterologous nucleic acid is operably linked to a promoter; and b) a pharmaceutically acceptable excipient. In some embodiments, the heterologous nucleic acid encodes the immune checkpoint inhibitor, the immunoactivator, and the CAR. In some embodiments, the heterologous nucleic acid encodes at least two immunoactivators. In some embodiments, the engineered mammalian immune cell is a PBMC, a T cell, or an NK cell. In some embodiments, the promoter is a constitutive promoter, such as hEF1α promoter. In some embodiments, the promoter is inducible. In some embodiments, the promoter is a T cell activation dependent promoter, such as an IL-2 promoter, an NFAT promoter, or an NFκB promoter. In some embodiments, the immune checkpoint inhibitor is an inhibitor of an immune checkpoint molecule selected from the group consisting of PD-1, PD-L1, PD-L2, CTLA-4, BLTA, TIM-3, or LAG-3. In some embodiments, the immune checkpoint inhibitor is an antibody (such as full-length antibody, scFv, single-domain antibody, heavy chain-only antibody, or Fab). In some embodiments, the immunoactivator is selected from the group consisting of IL-2, IL-7, IL-15, IL-21, IL-12, CCR4, CCR2b, Heparanase, CD137L, LEM, and Bcl-2. In some embodiments, the CAR targets a tumor antigen, such as EGFR, e.g., EGFRvIII, BCMA, or NY-ESO-1. In some embodiments, the CAR comprises an intracellular signaling domain having an abolished or attenuated immune effector function. In some embodiments, the CAR is a truncated CAR. In some embodiments, the CAR does not comprise a primary intracellular signaling domain (such as CD3ζ). In some embodiments, the CAR comprises a nonfunctional or attenuated primary intracellular signaling domain (such as a mutant CD3ζ). In some embodiments, the CAR alone does not induce cytolysis of the target cells. In some embodiments, the engineered mammalian immune cell is obtained from the individual. In some embodiments, the engineered mammalian immune cell is allogenic to the individual.

In some embodiments, there is provided a method of treating a cancer in an individual (such as a human individual), comprising administering to the individual an effective amount of a pharmaceutical composition comprising a) an engineered mammalian cell comprising a heterologous nucleic acid encoding an immunomodulator, wherein the heterologous nucleic acid is operably linked to a promoter; b) a second mammalian immune cell expressing a chimeric antigen receptor (CAR) or a recombinant T cell receptor (TCR); and c) a pharmaceutically acceptable excipient. In some embodiments, the engineered mammalian cell is an immune cell (such as a PBMC, an NK cell, or a T cell). In some embodiments, the engineered mammalian cell is a stem cell. In some embodiments, the promoter is inducible by an inducing condition selected from inducer (such as small molecule, for example, tetracycline, or doxycycline), irradiation, temperature, redox state, tumor environment, and the activation state of the engineered mammalian cell. In some embodiments, the immunomodulator is an immune checkpoint inhibitor (such as an inhibitor of CTLA-4, or an inhibitor of PD-1). In some embodiments, the immunomodulator is an immunoactivator. In some embodiments, the immunomodulator is a secreted protein. In some embodiments, the immunomodulator is an antibody (such as full-length antibody, scFv, single-domain antibody, heavy chain-only antibody, or Fab). In some embodiments, the engineered mammalian cell further expresses on its surface a targeting molecule recognizing a tumor antigen. In some embodiments, the engineered mammalian c ell further comprises a second heterologous nucleic acid encoding a therapeutic protein (such as a second immunomodulator, for example, an immunoactivator; or a therapeutic protein that is not an immunomodulator, for example chemotherapeutic antibody). In some embodiments, the immunoactivator is selected from the group consisting of IL-2, IL-7, IL-15, IL-21, IL-12, CCR4, CCR2b, Heparanase, CD137L, LEM, and Bcl-2. In some embodiments, the second mammalian immune cell is a PBMC, a T cell or an NK cell. In some embodiments, the CAR or TCR targets a tumor antigen, such as EGFR, e.g., EGFRvIII, BCMA, or NY-ESO-1. In some embodiments, the engineered mammalian cell and/or the second mammalian immune cell is obtained from the individual. In some embodiments, the engineered mammalian cell and/or the second mammalian immune cell is allogenic to the individual.

In some embodiments, there is provided a method of treating a cancer in an individual (such as a human individual), comprising a) administering to the individual an effective amount of a pharmaceutical composition comp rising an engineered mammalian cell comprising a heterologous nucleic acid encoding an immunomodulator, wherein the heterologous nucleic acid is operably linked to a promoter; and b) administering to the individual an effective amount of a pharmaceutical composition comprising a second mammalian immune cell expressing a chimeric antigen receptor (CAR) or a recombinant T cell receptor (TCR). In some embodiments, the pharmaceutical composition comprising the engineered mammalian cell is administered prior to the administration of the pharmaceutical composition comprising the second engineered mammalian immune cell. In some embodiments, the pharmaceutical composition comprising the engineered mammalian cell is administered after the administration of the pharmaceutical composition comprising the second engineered mammalian immune cell. In some embodiments, the engineered mammalian cell is an immune cell (such as a PBMC, an NK cell, or a T cell). In some embodiments, the engineered mammalian cell is astern cell. In some embodiments, the promoter is inducible by an inducing condition selected from inducer (such as small molecule, for example, tetracycline, or doxycycline), irradiation, temperature, redox state, tumor environment, and the activation state of the engineered mammalian cell. In some embodiments, the immunomodulator is an immune checkpoint inhibitor (such as an inhibitor of CTLA-4, or an inhibitor of PD-1). In some embodiments, the immunomodulator is an immunoactivator. In some embodiments, the immunomodulator is a secreted protein. In some embodiments, the immunomodulator is an antibody (such as full-length antibody, scFv, single-domain antibody, heavy chain-only antibody, or Fab). In some embodiments, the engineered mammalian cell further expresses on its surface a targeting molecule recognizing a tumor antigen. In some embodiments, the engineered mammalian cell further comprises a second heterologous nucleic acid encoding a therapeutic protein (such as a second immunomodulator, for example, an immunoactivator; or a therapeutic protein that is net an immunomodulator, for example, chemotherapeutic antibody). In some embodiments, the immunoactivator is selected from the group consisting of IL-2, IL-7, IL-15, IL-21, IL-12, CCR4, CCR2b, Heparanase, CD137L, LEM, and Bcl-2. In some embodiments, the second mammalian immune cell is a PBMC, a T cell or an NK cell. In some embodiments, the CAR or TCR targets a tumor antigen, such as EGFR, e.g., EGFRvIII, BCMA, or NY-ESO-1. In some embodiments, the engineered mammalian cell and/or the second mammalian immune cell is obtained from the individual. In some embodiments, the engineered mammalian cell and/or the second mammalian immune cell is allogenic to the individual.

The methods described herein are suitable for treating various cancers, including both solid cancer and liquid cancer. The methods are applicable to cancers of all stages, including early stage, advanced stage and metastatic cancer. The methods described herein may be used as a first therapy, second therapy, third therapy, or combination therapy with other types of cancer therapies known in the art, such as chemotherapy, surgery, radiation, gene therapy, immunotherapy, bone marrow transplantation, stem cell transplantation, targeted therapy, cryotherapy, ultrasound therapy, photodynamic therapy, radio-frequency ablation or the like, in an adjuvant setting or a neo adjuvant setting.

In some embodiments, the cancer is a solid cancer. In some embodiments, the cancer is a liquid cancer, such as hematologic cancer. Examples of cancers that may be treated by the methods described herein include, but are not limited to, adenocortical carcinoma, agnogenic myeloid metaplasia, anal cancer, appendix cancer, astrocytoma (e.g., cerebellar and cerebral), basal cell carcinoma, bile duct cancer (e.g., extrahepatic), bladder cancer, bone cancer, (osteosarcoma and malignant fibrous histiocytoma), brain tumor (e.g., glioma, brain stem glioma, cerebellar or cerebral astrocytoma (e.g., pilocyticastrocytoma, diffuse astrocytoma, anaplastic (malignant) astrocytoma), malignant glioma, ependymoma, oligodenglioma, meningioma, craniopharyngioma, haemangioblastomas, medulloblastoma, supratertorial primitive neuroectodermal tumors, visual pathway and hypothalamic glioma, and glioblastoma), breast cancer, bronchial adenomas/carcinoids, carcinoid tumor (e.g., gastrointestinal carcinoid tumor), carcinoma of unknown primary, central nervous system lymphoma, cervical cancer, colon cancer, colorectal cancer, chronic myeloproliferative disorders, endometria 1 cancer (e.g., uterine cancer), ependymoma, esophageal cancer, Ewing's family of tumors, eye cancer (e.g., intraocular melanoma and retinoblastoma), gallbladder cancer, gastric (stomach) cancer, gastrointestinal carcinoid tumor, gastrointestinal stromal tumor (GIST), germ cell tumor, (e.g., extracranial, extragonadal, ovarian), gestational trophoblastic tumor, head and neck cancer, hepatocellular (liver) cancer (e.g., hepatic carcinoma and heptoma), hypopharyngeal cancer, islet cell carcinoma (endocrine pancreas), laryngeal cancer, laryngeal cancer, leukemia (except for T-cell leukemia), lip and oral cavity cancer, oral cancer, liver cancer, lung cancer (e.g., small cell lung cancer, non-small cell lung cancer, adenocarcinoma of the lung and squamous carcinoma of the lung), lymphoma (except for T-cell lymphoma), medulloblastoma, melanoma, mesothelioma, metastatic squamous neck cancer, mouth cancer, multiple endocrine neoplasia syndrome, myelodysplastics syndromes, myelodysplastic/myeloproliferative diseases, nasal cavity and paranasal sinus cancer, nasopharyngeal carcinoma, neuroblastoma, neuroendocrine cancer, oropharyngeal cancer, ovarian cancer (e.g., ovarian epithelial cancer, ovarian germ cell tumor, ovarian low malignant potential tumor), pancreatic cancer, parathyroid cancer, penile cancer, cancer of the peritoneal, pharyngeal cancer, pheochromocytoma, pineoblastoma and supratentorial primitive neuroectodermal tumors, pituitary tumor, pleuropulmonary blastoma, primary central nervous system lymphoma (microglioma), pulmonary lymphangiomyomatosis, rectal cancer, renal carcinoma, renal pelvis and ureter cancer (transitional cell cancer), rhabdomyosarcoma, salivary gland cancer, skin cancer (e.g., non-melanoma (e.g., squamous cell carcinoma), melanoma, and Merkel cell carcinoma), small intestine cancer, squamous cell cancer, testicular cancer, throat cancer, thyroid cancer, tuberous sclerosis, urethral cancer, vaginal cancer, vulvar cancer, Wilms' tumor, abnormal vascular proliferation associated with phakomatoses, edema (such as that associated with brain tumors), and Meigs' syndrome.

Administration of the pharmaceutical compositions may be carried out in any convenient manner, including by injection, ingestion, transfusion, implantation or transplantation. The compositions may be administered to a patent transarterially, subcutaneously, intradermally, intratumorally, intranodally, intramedullary, intramuscularly, intravenously, or intraperitoneally. In some embodiments, the pharmaceutical composition is administered systemically. In some embodiments, the pharmaceutical composition is administered to an individual by infusion, such as intravenous infusion. Infusion techniques for immunotherapy are known in the art (see, e.g., Rosenberg et al., New Eng J. of Med. 319: 1676 (1988)). In some embodiments, the pharmaceutical composition is administered to an individual by intradermal or subcutaneous injection. In one embodiment, the compositions are administered by intravenous injection. In one embodiment, the compositions are injected directly into a tumor, or a lymph node. In some embodiments, the pharmaceutical composition is administered locally to a site of tumor, such as directly into tumor cells, or to a tissue having tumor cells.

In some embodiments, there is provided a method of treating a solid cancer in an individual (such as a human individual), comprising administering (such as systemically, or locally to a tumor site) to the individual an effective amount of a pharmaceutical composition comprising: a) an engineered mammalian cell comprising a heterologous nucleic acid encoding an immunomodulator, wherein the heterologous nucleic acid is operably linked to a promoter; and b) a pharmaceutically acceptable excipient. In some embodiments, the engineered mammalian cell is an immune cell (such as a PBMC, an NK cell, or a T cell). In some embodiments, the engineered mammalian cell is a stem cell. In some embodiments, the promoter is inducible by an inducing condition selected from inducer (such as small molecule, for example, tetracycline, or doxycycline), irradiation, temperature, redox state, tumor environment, and the activation state of the engineered mammalian cell. In some embodiments, the immunomodulator is an immune checkpoint inhibitor (such as an inhibitor of CTLA-4, or an inhibitor of PD-1). In some embodiments, the immunomodulator is an immunoactivator. In some embodiments, the immunomodulator is a secreted protein. In some embodiments, the immunomodulator is an antibody (such as full-length antibody, scFv, single-domain antibody, heavy chain-only antibody, or Fab). In some embodiments, the engineered mammalian cell further expresses on its surface a targeting molecule recognizing a tumor antigen (such as EGFRvIII). In some embodiments, the engineered mammalian cell does not express a CAR or a TCR. In some embodiments, the engineered mammalian cell further comprises a second heterologous nucleic acid encoding a therapeutic protein (such as a second immunomodulator, for example, an immuno activator; or a therapeutic protein that is not an immunomodulator, for example, chemotherapeutic antibody). In some embodiments, the immunoactivator is selected from the group consisting of IL-2, IL-7, IL-15, IL-21, IL-12, CCR4, CCR2b, Heparanase, CD137L, LEM, and Bcl-2. In some embodiments, the engineered mammalian cell is obtained from the individual. In some embodiments, the engineered mammalian cell is allogenic to the individual. In some embodiments, the solid cancer is selected from the group consisting of melanoma, breast cancer, lung cancer, liver cancer, leukemia, lymphoma, gastric cancer, colon cancer, bone cancer, brain cancer, pancreatic cancer, and ovarian cancer. In some embodiments, the pharmaceutical composition is administered by infusion. In some embodiments, the pharmaceutical composition is administered by intratumoral injection.

In some embodiments, there is provided a method of treating a solid cancer in an individual (such as a human individual), comprising administering (such as systemically, or locally to a tumor site) to the individual an effective amount of a pharmaceutical composition comprising: a) an engineered mammalian (such as human) immune cell comprising a heterologous nucleic acid encoding an immuno modulator, wherein the heterologous nucleic acid is operably linked to a promoter, wherein the engineered mammalian immune cell further expresses a CAR or TCR; and b) a pharmaceutically acceptable excipient. In some embodiments, the engineered mammalian immune cell is a PBMC, a T cell, or an NK cell. In some embodiments, the promoter is inducible, such as by the intracellular signaling domain of the CAR or TCR. In some embodiments, the promoter is a T cell activation dependent promote, such as an IL-2 promoter, an NFAT promoter, or an NFκB promoter. In some embodiments, the immunomodulator is an immune checkpoint inhibitor (such as an inhibitor of CTLA-4, or an inhibitor of PD-1). In some embodiments, the immunomodulator is an immuno activator. In some embodiments, the immunomodulator is a secreted protein. In some embodiments, the immunomodulator is an antibody (such as full-length antibody, scFv, single-domain antibody, heavy chain-only antibody, or Fab). In some embodiments, the engineered mammalian cell further comprises a second heterologous nucleic acid encoding a therapeutic protein (such as a second immunomodulator, for example, an immunoactivator; or a therapeutic protein that is not an immunomodulator, for example, chemotherapeutic antibody, e.g., an anti-HER2 antibody). In some embodiments, the immunoactivator is selected from the group consisting of IL-2, IL-15, IL-21, IL-12, CCR4, CCR2b, Heparanase, CD137L, LEM, and Bcl-2. In some embodiments, the CAR is encoded by a third heterologous nucleic acid operably linked to a second promoter. In some embodiment, the second promoter is a constitutive promoter. In some embodiments, the second promoter is inducible, for example, by an inducing condition selected from inducer (such as small molecule, for example, tetracycline, or doxycycline), irradiation, temperature, redox state, tumor environment, and the activation state of the engineered mammalian immune cell. In some embodiments, the CAR or TCR targets a tumor antigen, such as EGFRvIII. In some embodiments, the engineered mammalian immune cell is obtained from the individual. In some embodiments, the engineered mammalian immune cell is allogenic to the individual. In some embodiments, the solid cancer is selected from the group consisting of melanoma, breast cancer, lung cancer, liver cancer, leukemia, lymphoma, gastric cancer, colon cancer, bone cancer, bra in cancer, pancreatic cancer, and ovarian cancer. In some embodiments, the pharmaceutical composition is administered by infusion. In some embodiments, the pharmaceutical composition is administered by intratumoral injection.

In some embodiments, there is provided a method of treating a solid cancer in an individual (such as a human individual), comprising administering (such as systemically, or locally to a tumor site) to the individual an effective amount of a pharmaceutical composition, comprising: a) an engineered mammalian cell comprising a heterologous nucleic acid encoding an immunomodulator, wherein the heterologous nucleic acid is operably linked to a promoter; b) a second mammalian immune cell expressing a chimeric antigen receptor (CAR) or a recombinant T cell receptor (TCR); and c) a pharmaceutically acceptable excipient. In some embodiments, the engineered mammalian cell is an immune cell (such as a PBMC, an NK cell, or a T cell). In some embodiments, the engineered mammalian cell is a stem cell. In some embodiments, the promoter is inducible by an inducing condition selected from inducer (such as small molecule, for example tetracycline, or doxycycline), irradiation, temperature, redox state, tumor environment, and the activation state of the engineered mammalian cell. In some embodiments, the immunomodulator is an immune checkpoint inhibitor (such as an inhibitor of CTLA-4, or an inhibitor of PD-1). In some embodiments, the immunomodulator is an immunoactivator. In some embodiments, the immunomodulator is a secreted protein. In some embodiments, the immunomodulator is an antibody (such as full-length antibody, scFv, singe-domain antibody, heavy chain-only antibody, or Fab). In some embodiments, the engineered mammalian cell further expresses on its surface a targeting molecule recognizing a tumor antigen. In some embodiments, the engineered mammalian cell further comprises a second heterologous nucleic acid encoding a therapeutic protein (such as a second immuno modulator, for example, an immunoactivator; or a therapeutic protein that is not an immunomodulator, for example, chemotherapeutic antibody). In some embodiments, the immunoactivator is selected from the group consisting of IL-2, IL-7, IL-15, IL-21, IL-12, CCR4, CCR2b, Heparanase, CD137L, LEM, and Bcl-2. In some embodiments, the second mammalian immune cell is a PBMC, a T cell or an NK cell. In some embodiments, the CAR or TCR targets a tumor antigen, such as EGFRvIII. In some embodiments, the engineered mammalian cell and/or the second mammalian immune cell is obtained from the individual. In some embodiments, the engineered mammalian cell and/or the second mammalian immune cell is allogenic to the individual. In some embodiments, the solid cancer is selected from the group consisting of melanoma, breast cancer, lung cancer, liver cancer, leukemia, lymphoma, gastric cancer, colon cancer, bone cancer, brain cancer, pancreatic cancer, and ovarian cancer. In some embodiments, the pharmaceutical composition is administered by infusion. In some embodiments, the pharmaceutical composition is administered by intratumoral injection.

In some embodiments, there is provided a method of treating a solid cancer in an individual (such as a human individual), comprising: a) ad ministering to the individual an effective amount of a pharmaceutical composition comp rising an engineered mammalian cell comprising a heterologous nucleic acid encoding an immunomodulator, wherein the heterologous nucleic acid is operably linked to a promoter; and b) administering to the individual an effective amount of a pharmaceutical composition comprising a second mammalian immune cell expressing a chimeric antigen receptor (CAR) or a recombinant T cell receptor (TCR). In some embodiments, the pharmaceutical composition comprising the engineered mammalian cell is administered prior to the administration of the pharmaceutical composition comprising the second engineered mammalian immune cell. In some embodiments, the pharmaceutical composition comprising the engineered mammalian cell is administered after the administration of the pharmaceutical composition comprising the second engineered mammalian immune cell. In some embodiments, the engineered mammalian cell is an immune cell (such as a PBMC, an NK cell, or a T In some embodiments, the engineered mammalian cell is a stem (211. In some embodiments, the promoter is inducible by an inducing condition selected from inducer (such as small molecule, for example, tetracycline, or doxycycline), irradiation, temperature, redox state, tumor environment, and the activation state of the engineered mammalian cell. In some embodiments, the immunomodulator is an immune checkpoint inhibitor (such as an inhibitor of CTLA-4, or an inhibitor of PD-1). In some embodiments, the immunomodulator is an immunoactivator. In some embodiments, the immunomodulator is a secreted protein. In some embodiments, the immunomodulator is an antibody (such as full-length antibody, scFv, singe-domain antibody, heavy chain-only antibody, or Fab). In some embodiments, the engineered mammalian cell further expresses on its surface a targeting molecule recognizing a tumor antigen. In some embodiments, the engineered mammalian cell further comprises a second heterologous nucleic acid encoding a therapeutic protein (such as a second immuno modulator, for example, an immunoactivator: or a therapeutic protein that is not an immunomodulator, for example, chemotherapeutic antibody). In some embodiments, the immunoactivator is selected from the group consisting of IL-2, IL-7, IL-15, IL-21, IL-12, CCR4, CCR2b, Heparanase, CD137L, LEM, and Bcl-2. In some embodiments, the second mammalian immune cell is a PBMC, a T cell or an NK cell. In some embodiments, the CAR or TCR targets a tumor antigen, such as EGFRvIII. In some embodiments, the engineered mammalian cell and/or the second mammalian immune cell is obtained from the individual. In some embodiments, the engineered mammalian cell and/or the second mammalian immune cell is allogenic to the individual. In some embodiments, the solid cancer is selected from the group consisting of melanoma, breast cancer, lung cancer, liver cancer, leukemia, lymphoma, gastric cancer, colon cancer, bone cancer, brain cancer, pancreatic cancer, and ovarian cancer. In some embodiments, the pharmaceutical composition is administered by infusion. In some embodiments, the pharmaceutical composition is administered by intratumoral injection.

In some embodiments, there is provided a method of treating a liquid cancer in an individual (such as a human individual), comprising systemically administering to the individual an effective amount of a pharmaceutical composition comprising a) an engineered mammalian cell comprising a heterologous nucleic acid encoding an immuno modulator, wherein the heterologous nucleic acid is operably linked to a promoter; and b) a pharmaceutically acceptable excipient. In some embodiments, the engineered mammalian cell is an immune cell (such as a PBMC, an NK cell, or a T cell). In some embodiments, the engineered mammalian cell is a stem cell. In some embodiments, the promoter is inducible by an inducing condition selected from inducer (such as small molecule, for example, tetracycline, or doxycycline), irradiation, temperature, redox state, tumor environment, and the activation state of the engineered mammalian cell. In some embodiments, the immunomodulator is an immune checkpoint inhibitor (such as an inhibitor of CTLA-4, or an inhibitor of PD-1). In some embodiments, the immunomodulator is an immunoactivator. In some embodiments, the immunomodulator is a secreted protein. In some embodiments, the immunomodulator is an antibody (such as full-length antibody, scFv, single-domain antibody, heavy chain-only antibody, or Fab). In some embodiments, the engineered mammalian cell further expresses on its surface a targeting molecule recognizing a tumor antigen, such as BCMA, or NY-E90-1. In some embodiments, the engineered mammalian cell does not express a CAR or a TCR. In some embodiments, the engineered mammalian cell further comprises a second heterologous nucleic acid encoding a therapeutic protein (such as a second immunomodulator, for example, an immuno activator; or a therapeutic protein that is not an immunomodulator, for example, chemotherapeutic antibody). In some embodiments, the immunoactivator is selected from the group consisting of IL-2, IL-7, IL-15, IL-21, IL-12, CCR4, CCR2b, Heparanase, CD137L, LEM, and Bcl-2. In some embodiments, the engineered mammalian cell is obtained from the individual. In some embodiments, the engineered mammalian cell is allogenic to the individual. In some embodiments, the liquid cancer is leukemia or lymphoma. In some embodiments, the pharmaceutical composition is administered by infusion.

In some embodiments, there is provided a method of treating a liquid cancer in an individual (such as a human individual), comprising systemically administering to the individual an effective amount of a pharmaceutical composition comprising: a) an engineered mammalian (such as human) immune cell comprising a heterologous nucleic acid encoding an immunomodulator, wherein the heterologous nucleic acid is operably linked to a promoter, wherein the engineered mammalian immune cell further expresses a CAR or TCR; and b) a pharmaceutically acceptable excipient. In some embodiments, the engineered mammalian immune cell is a PBMC, a T cell, or an NK cell. In some embodiments, the promoter is inducible, such as by the intracellular signaling domain of the CAR or TCR In some embodiments, the promoter is a T cell activation dependent promoter, such as an IL-2 promoter, an NFAT promoter, or an NFκB promoter. In some embodiments, the immunomodulator is an immune checkpoint inhibitor (such as an inhibitor of CTLA-4, or an inhibitor of PD-1). In some embodiments, the immunomodulator is an immunoactivator. In some embodiments, the immunomodulator is a secreted protein. In some embodiments, the immunomodulator is an antibody (such as full-length antibody, scFv, single-domain antibody, heavy chain-only antibody, or Fab). In some embodiments, the engineered mammalian cell further comprises a second heterologous nucleic acid encoding a therapeutic protein (such as a second immunomodulator, for example, an immunoactivator; or a therapeutic protein that is not an immunomodulator, for example, chemotherapeutic antibody). In some embodiments, the immunoactivator is selected from the group consisting of IL-2, IL-7, IL-15, IL-21, IL-12, CCR4, CCR2b, Heparanase, CD137L, LEM, and Bcl-2. In some embodiments, the CAR is encoded by a third heterologous nucleic acid operably linked to a second promoter. In some embodiment, the second promoter is a constitutive promoter. In some embodiments, the second promoter is inducible, for example, by an inducing condition selected from inducer (such as small molecule, for example, tetracycline, or doxycycline), irradiation, temperature, redox state, tumor environment, and the activation state of the engineered mammalian immune cell. In some embodiments, the CAR or TCR targets a tumor antigen, such as BCMA, or NY-ESO-1. In some embodiments, the engineered mammalian immune cell is obtained from the individual. In some embodiments, the engineered mammalian immune cell is allogenic to the individual. In some embodiments, the liquid cancer is leukemia or lymphoma. In some embodiments, the pharmaceutical composition is administered by infusion.

In some embodiments, there is provided a method of treating a liquid cancer in an individual (such as a human individual), comprising systemically administering to the individual an effective amount of a pharmaceutical composition, comprising: a) an engineered mammalian cell comprising a heterologous nucleic acid encoding an immunomodulator, wherein the heterologous nucleic acid is operably linked to a promoter; b) a second mammalian immune cell expressing a chimeric antigen receptor (CAR) or a recombinant T cell receptor (TCR); and c) a pharmaceutically acceptable excipient. In some embodiments, the engineered mammalian cell is an immune cell (such as a PBMC, an NK cell, or a T cell). In some embodiments, the engineered mammalian cell is a stem cell. In some embodiments, the promoter is inducible by an inducing condition selected from inducer (such as small molecule, for example, tetracycline, or doxycycline), irradiation, temperature, redox state, tumor environment, and the activation state of the engineered mammalian cell. In some embodiments, the immunomodulator is an immune checkpoint inhibitor (such as an inhibitor of CTLA-4, or an inhibitor of PD-1). In some embodiments, the immunomodulator is an immunoactivator. In some embodiments, the immunomodulator is a secreted protein. In some embodiments, the immunomodulator is an antibody (such as full-length antibody, scFv, single-domain antibody, heavy chain-only antibody, or Fab). In some embodiments, the engineered mammalian cell further expresses on its surface a targeting molecule recognizing a tumor antigen. In some embodiments, the engineered mammalian c ell further comprises a second heterologous nucleic acid encoding a therapeutic protein (such as a second immunomodulator, for example, an immunoactivator; or a therapeutic protein that is not an immunomodulator, for example chemotherapeutic antibody). In some embodiments, the immunoactivator is selected from the group consisting of IL-2, IL-7, IL-15, IL-21, IL-12, CCR4, CCR2b, Heparanase, CD137L, LEM, and Bcl-2. In some embodiments, the second mammalian immune cell is a PBMC, a T cell or an NK cell. In some embodiments, the CAR or TCR targets a tumor antigen, such as BCMA, or NY-ESO-1. In some embodiments, the engineered mammalian cell and/or the second mammalian immune cell is obtained from the individual. In some embodiments, the engineered mammalian cell and/or the second mammalian immune cell is allogenic to the individual. In some embodiments, the liquid cancer is leukemia or lymphoma. In some embodiments, the pharmaceutical composition is administered by infusion.

In some embodiments, there is provided a method of treating a liquid cancer in an individual (such as a human individual), comprising: a) systematically administering to the individual an effective amount of a pharmaceutical composition comprising an engineered mammalian cell comprising a heterologous nucleic acid encoding an immuno modulator, wherein the heterologous nucleic acid is operably linked to a promoter; and b) systematically administering to the individual an effective amount of a pharmaceutical composition comprising a second mammalian immune cell expressing a chimeric antigen receptor (CAR) or a recombinant T cell receptor (TCR). In so me embodiments, the pharmaceutical composition comprising the engineered mammalian cell is administered prior to the administration of the pharmaceutical composition comprising the second engineered mammalian immune cell. In some embodiments, the pharmaceutical composition comprising the engineered mammalian cell is administered after the administration of the pharmaceutical composition comprising the second engineered mammalian immune cell. In some embodiments, the engineered mammalian cell is an immune cell (such as a PBMC, an NK cell, or a T cell). In some embodiments, the engineered mammalian cell is a stem cell. In some embodiments, the promoter is inducible by an inducing condition selected from inducer (such as small molecule, for example, tetracycline, or doxycycline), irradiation, temperature, redox state, tumor environment, and the activation state of the engineered mammalian cell. In some embodiments, the immunomodulator is an immune checkpoint inhibitor (such as an inhibitor of CTLA-4, or an inhibitor of PD-1). In some embodiments, the immunomodulator is an immunoactivator. In some embodiments, the immunomodulator is a secreted protein. In some embodiments, the immunomodulator is an antibody (such as full-length antibody, scFv, single-domain antibody, heavy chain-only antibody, or Fab). In some embodiments, the engineered mammalian cell further expresses on its surface a targeting molecule recognizing a tumor antigen. In some embodiments, the engineered mammalian cell further comprises a second heterologous nucleic acid encoding a therapeutic protein (such as a second immunomodulator, for example, an immunoactivator; or a therapeutic protein that is not an immunomodulator, for example, chemotherapeutic antibody). In some embodiments, the immunoactivator is selected from the group consisting of IL-2, IL-7, IL-15, IL-21, IL-12, CCR4, CCR2b, Heparanase, CD137L, LEM, and Bcl-2. In some embodiments, the second mammalian immune cell is a PBMC, a T cell or an NK cell. In some embodiments, the CAR or TCR targets a tumor antigen, such as BCMA, or NY-ESO-1. In some embodiments, the engineered mammalian cell and/or the second mammalian immune cell is obtained from the individual. In some embodiments, the engineered mammalian cell and/or the second mammalian immune cell is allogenic to the individual. In some embodiments, the liquid cancer is leukemia or lymphoma. In some embodiments, the pharmaceutical composition is administered by infusion.

In some embodiments, wherein the promoter is inducible, the method further comprises inducing the expression of the immunomodulator and/or other therapeutic proteins. The engineered mammalian cell and/or the second mammalian immune cell can be induced prior to administration to the individual, or after administration to the individual. In some embodiments, wherein the promoter is inducible by an inducing condition, the method further comprises applying the inducing condition to the individual. In some embodiments, wherein the promoter is inducible by an inducer (such as a small molecule inducer, for example, tetracycline or doxycycline), the method further comprises administering to the individual an effective amount of the inducer to induce the expression of the immunomodulator and/or other therapeutic proteins. In some embodiments, the inducer is administered systemically. In some embodiments, the inducer is administered locally to a site of tumor, such as directly into the tumor cells, or to a tissue having tumor cells. In some embodiments, wherein the promoter is inducible by irradiation (such as light or ionizing radiation), the method further comprises applying irradiation to the individual, such as to the whole body or locally to a tumor site. In some embodiments, wherein the promoter is inducible by heat, the method further comprises applying heat to the individual, such as locally to a tumor site.

Thus, in some embodiments, there is provided a method of treating a cancer in an individual (such as a human individual), comprising: (1) administering to the individual an effective amount of a pharmaceutical composition comprising a) an engineered mammalian cell comprising a heterologous nucleic acid encoding an immuno modulator, wherein the heterologous nucleic acid is operably linked to an inducible promoter; and b) a pharmaceutically acceptable excipient; and (2) inducing the expression of the immunomodulator. In some embodiments, the engineered mammalian cell is an immune cell (such as a PBMC, an NK cell, or a T cell). In some embodiments, the engineered mammalian c ell is a stem cell. In some embodiments, the promoter is inducible by an inducing condition selected from inducer (such as small molecule, for example, tetracycline, or doxycycline), irradiation, temperature, redox state, tumor environment, and the activation state of the engineered mammalian cell. In some embodiments, the immunomodulator is an immune checkpoint inhibitor (such as an inhibitor of CTLA-4, or an inhibitor of PD-1). In some embodiments, the immunomodulator is an immunoactivator. In some embodiments, the immunomodulator is a secreted protein. In some embodiments, the immunomodulator is an antibody (such as full-length antibody, scFv, singe-domain antibody, heavy chain-only antibody, or

Fab). In some embodiments, the engineered mammalian cell further expresses on its surface a targeting molecule recognizing a tumor antigen (such as EGFR, e.g., EGFRvIII, BCMA, or NY-ESO-1). In some embodiments, the engineered mammalian cell does not express a CAR or a TCR. In some embodiments, the engineered mammalian cell further comprises a second heterologous nucleic acid encoding a therapeutic protein (such as a second immunomodulator, for example, an immunoactivator; or a therapeutic protein that is not an immunomodulator, for example chemotherapeutic antibody). In some embodiments, the immunoactivator is selected from the group consisting of IL-2, IL-7, IL-15, IL-21, IL-12, CCR4, CCR2b, Heparanase, CD137L, LEM, and Bcl-2. In some embodiments, the engineered mammalian cell is obtained from the individual. In some embodiments, the engineered mammalian cell is allogenic to the individual. In some embodiments, the cancer is selected from the group consisting of leukemia, lymphoma, melanoma, breast cancer, lung cancer, liver cancer, leukemia, lymphoma, gastric cancer, colon cancer, bone cancer, brain cancer, pancreatic cancer, and ovarian cancer. In some embodiments, the pharmaceutical composition is administered systemically (such as by infusion), and the promoter is induced locally at the tumor site (such as by local administration of an inducer, or by local heating or irradiation).

In some embodiments, there is provided a method of treating a cancer in an individual (such as a human individual), comprising (1) administering to the individual an effective amount of a pharmaceutical composition comprising a) an engineered mammalian (such as human) immune cell comprising a heterologous nucleic acid encoding an immunomodulator, wherein the heterologous nucleic acid is operably linked to an inducible promoter, wherein the engineered mammalian immune cell further expresses a CAR or TCR; and b) a pharmaceutically acceptable excipient; and (2) inducing the expression of the immunomodulator. In some embodiments, the engineered mammalian immune cell is a PBMC, a T cell, or an NK cell. In some embodiments, the promoter is inducible, such as by the intracellular signaling domain of the CAR or TCR. In some embodiments, the promoter is a T cell activation dependent promoter, such as an IL-2 promoter, an NFAT promoter, or an NFκB promoter. In some embodiments, the immunomodulator is an immune checkpoint inhibitor (such as an inhibitor of CTLA-4, or an inhibitor of PD-1). In some embodiments, the immunomodulator is an immunoactivator. In some embodiments, the immunomodulator is a secreted protein. In some embodiments, the immunomodulator is an antibody (such as full-length antibody, scFv, singe-domain antibody, heavy chain-only antibody, or Fab). In some embodiments, the engineered mammalian cell further comprises a second heterologous nucleic acid encoding a therapeutic protein (such as a second immunomodulator, or for example, an immunoactivator; or a therapeutic protein that is net an immunomodulator, for example, chemotherapeutic antibody, e.g., an anti-HER2 antibody). In some embodiments, the immunoactivator is selected from the group consisting of IL-2, IL-7, IL-15, IL-21, IL-12, CCR4, CCR2b, Heparanase, CD137L, LEM, and Bcl-2. In some embodiments, the CAR is encoded by a third heterologous nucleic acid operably linked to a second promoter. In some embodiment, the second promoter is a constitutive promoter. In some embodiments, the second promoter is inducible, for example, by an inducing condition selected from inducer (such as small molecule, for example, tetracycline, or doxycycline), irradiation, temperature, redox state, tumor environment, and the activation state of the engineered mammalian immune cell. In some embodiments, the CAR or TCR targets a tumor antigen, such as EGFR, e.g., EGFRvIII, BCMA, or NY-ESO-1. In some embodiments, the engineered mammalian immune cell is obtained from the individual. In some embodiments, the engineered mammalian immune cell is allogenic to the individual. In some embodiments, the cancer is selected from the group consisting of leukemia, lymphoma, melanoma, breast cancer, lung cancer, liver cancer, leukemia, lymphoma, gastric cancer, colon cancer, bone cancer, brain cancer, pancreatic cancer, and ovarian cancer. In some embodiments, the pharmaceutical composition is administered systemically (such as by infusion), and the promoter is induced locally at the tumor site (such as by local administration of an inducer, or by local heating or irradiation).

In some embodiments, there is provided a method of treating a cancer in an individual (such as a human individual), comprising (1) administering to the individual an effective amount of a pharmaceutical composition comprising a) an engineered mammalian cell comprising a heterologous nucleic acid encoding an immunomodulator, wherein the heterologous nucleic acid is operably linked to an inducible promoter; b) a second mammalian immune cell expressing a chimeric antigen receptor (CAR) or a recombinant T cell receptor (TCR); and c) a pharmaceutically acceptable excipient; and (2) inducing the expression of the immunomodulator. In some embodiments, the engineered mammalian cell is an immune cell (such as a PBMC, an NK cell, or a T cell). In some embodiments, the engineered mammalian cell is a stem cell. In some embodiments, the promoter is inducible by an inducing condition selected from inducer (such as small molecule, for example, tetracycline, or doxycycline), irradiation, temperature, redox state, tumor environment, and the activation state of the engineered mammalian cell. In some embodiments, the immunomodulator is an immune checkpoint inhibitor (such as an inhibitor of CTLA-4, or an inhibitor of PD-1). In some embodiments, the immunomodulator is an immunoactivator. In some embodiments, the immunomodulator is a secreted protein. In some embodiments, the immunomodulator is an antibody (such as full-length antibody, scFv, single-domain antibody, heavy chain-only antibody, or Fab). In some embodiments, the engineered mammalian cell further expresses on its surface a targeting molecule recognizing a tumor antigen. In some embodiments, the engineered mammalian cell further comprises a second heterologous nucleic acid encoding a therapeutic protein (such as a second immunomodulator, for example, an immunoactivator; or a therapeutic protein that is not an immunomodulator, for example chemotherapeutic antibody). In some embodiments, the immunoactivator is selected from the group consisting of IL-2, IL-7, IL-15, IL-21, IL-12, CCR4, CCR2b, Heparanase, CD137L, LEM, and Bcl-2. In some embodiments, the second mammalian immune cell is a PBMC, a T cell or an NK cell. In some embodiments, the CAR or TCR targets a tumor antigen, such as EGFR, e.g., EGFRvIII, BCMA, or NY-ESO-1. In some embodiments, the engineered mammalian cell and/or the second mammalian immune cell is obtained from the individual. In some embodiments, the engineered mammalian cell and/or the second mammalian immune cell is allogenic to the individual. In some embodiments, the cancer is selected from the group consisting of leukemia, lymphoma, melanoma, breast cancer, lung cancer, liver cancer, leukemia, lymphoma, gastric cancer, colon cancer, bone cancer, brain cancer, pancreatic cancer, and ovarian cancer. In some embodiments, the pharmaceutical composition is administered systemically (such as by infusion), and the promoter is induced locally at the tumor site (such as by local administration of an inducer, or by local heating or irradiation).

In some embodiments, there is provided a method of treating a cancer in an individual (such as a human individual), comprising a) administering to the individual an effective amount of a pharmaceutical composition comp rising an engineered mammalian cell comprising a heterologous nucleic acid encoding an immunomodulator, wherein the heterologous nucleic acid is operably linked to an inducible promoter; b) administering to the individual an effective amount of a pharmaceutical composition comprising a second mammalian immune cell expressing a chimeric antigen receptor (CAR) or a recombinant T cell receptor (TCR); and c) inducing the expression of the immunomodulator. In some embodiments, the pharmaceutical composition comprising the engineered mammalian cell is administered prior to the administration of the pharmaceutical composition comprising the second engineered mammalian immune cell. In some embodiments, the pharmaceutical composition comprising the engineered mammalian cell is administered after the administration of the pharmaceutical composition comprising the second engineered mammalian immune cell. In some embodiments, the engineered mammalian cell is an immune cell (such as a PBMC, an NK cell, or a T cell). In some embodiments, the engineered mammalian cell is a stem cell. In some embodiments, the promoter is inducible by an inducing condition selected from inducer (such as small molecule, for example, tetracycline, or doxycycline), irradiation, temperature, redox state, tumor environment, and the activation state of the engineered mammalian cell. In some embodiments, the immunomodulator is an immune checkpoint inhibitor (such as an inhibitor of CTLA-4, or an inhibitor of PD-1). In some embodiments, the immunomodulator is an immunoactivator. In some embodiments, the immunomodulator is a secreted protein. In some embodiments, the immunomodulator is an antibody (such as full-length antibody, scFv, single-domain antibody, heavy chain-only antibody, or Fab). In some embodiments, the engineered mammalian cell further expresses on its surface a targeting molecule recognizing a tumor antigen. In some embodiments, the engineered mammalian c ell further comprises a second heterologous nucleic acid encoding a therapeutic protein (such as a second immunomodulator, for example, an immunoactivator; or a therapeutic protein that is not an immunomodulator, for example, chemotherapeutic antibody). In some embodiments, the immunoactivator is selected from the group consisting of IL-2, IL-7, IL-15, IL-21, IL-12, CCR4, CCR2b, Heparanase, CD137L, LEM, and Bcl-2. In some embodiments, the second mammalian immune cell is a PBMC, a T cell or an NK cell. In some embodiments, the CAR or TCR targets a tumor antigen, such as EGFR, e.g., EGFRvIII, BCMA, or NY-ESO-1. In some embodiments, the engineered mammalian cell and/or the second mammalian immune cell is obtained from the individual. In some embodiments, the engineered mammalian cell and/or the second mammalian immune cell is allogenic to the individual. In some embodiments, the cancer is selected from the group consisting of leukemia, lymphoma, melanoma, breast cancer, lung cancer, liver cancer, leukemia, lymphoma, gastric cancer, colon cancer, bone cancer, brain cancer, pancreatic cancer, and ovarian cancer. In some embodiments, the pharmaceutical composition comprising the engineered mammalian cell is administered systemically (such as by infusion), and the promoter is induced locally at the tumor site (such as by local administration of an inducer, or by local heating or irradiation).

In some embodiments, the pharmaceutical composition is administered at a dosage of at least about any of 10⁴, 10⁵, 10⁶, 10⁷, 10⁸, or 10⁹ cells/kg of body weight. In some embodiments, the pharmaceutical composition is administered at a dosage of any of about 10⁴ to about 10′, about 10⁵ to about 10⁶, about 10⁶ to about 10⁷, about 10′ to about 10³, about 10⁸ to about 10⁹, about 10⁴ to about 10⁹, about 10⁴ to about 10⁶, about 10⁶ to about 10³, or about 10⁵ to about 10′ cells/kg of body weight.

In some embodiments, wherein more than one type of engineered mammalian cells are administered, the different types of engineered mammalian cells may be administered to the individual simultaneously, such as in a single composition, or sequentially in any suitable order.

In some embodiments, the pharmaceutical composition is administered for a single time. In some embodiments, the pharmaceutical composition is administered for multiple times (such as any of 2, 3, 4, 5, 6, or more times) In some embodiments, the pharmaceutical composition is administered once per week, once 2 weeks, once 3 weeks, once 4 weeks, once per month, once per 2 months, once per 3 months, once per 4 months, once per 5 months, once per 6 months, once per 7 months, once per 8 months, once per 9 months, or once per year. In some embodiments, the interval between administrations is about any one of 1 week to 2 weeks, 2 weeks to 1 month, 2 weeks to 2 months, 1 month to 2 months, 1 month to 3 months, 3 months to 6 months, or 6 months to a year. The optimal dos age and treatment regime for a particular patient can readily be determined by one skilled in the art of medicine by monitoring the patient for signs of disease and adjusting the treatment accordingly.

### V. Kits and Articles of Manufacture

Further provided are kits, unit dosages, and articles of manufacture comprising any of the pharmaceutical compositions described herein.

In some embodiments, there is provided a kit comprising: (1) a pharmaceutical composition comprising: a) an engineered mammalian cell comprising a heterologous nucleic acid encoding an immuno modulator, wherein the heterologous nucleic acid is operably linked to a promoter; and b) a pharmaceutically acceptable excipient; and (2) an instruction for using the pharmaceutical composition. In some embodiments, the engineered mammalian cell is a stem cell. In some embodiments, the promoter is inducible by an inducing condition selected from inducer (such as small molecule, for example, tetracycline, or doxycycline), irradiation, temperature, redox state, tumor environment, and the activation state of the engineered mammalian cell. In some embodiments, the immunomodulator is an immune checkpoint inhibitor (such as an inhibitor of CTLA-4, or an inhibitor of PD-1). In some embodiments, the immunomodulator is an immunoactivator. In some embodiments, the immunomodulator is a secreted protein. In some embodiments, the immunomodulator is an antibody (such as full-length antibody, scFv, single-domain antibody, heavy chain-only antibody, or Fab). In some embodiments, the engineered mammalian cell further expresses on its surface a targeting molecule recognizing a tumor antigen (such as EGFR, e.g., EGFRvIII, BCMA, or NY-ESO-1). In some embodiments, the engineered mammalian cell does not express a CAR or a TCR. In some embodiments, the engineered mammalian cell further comprises a second heterologous nucleic acid encoding a therapeutic protein (such as a second immunomodulator, for example, an immunoactivator; or a therapeutic protein that is not an immunomodulator, for example, chemotherapeutic antibody). In some embodiments, the immunoactivator is selected from the group consisting of IL-2, IL-7, IL-15, IL-21, IL-12, CCR4, CCR2b, Heparanase, CD137L, LEM, and Bcl-2. In some embodiments, the kit further comprises an inducer.

In some embodiments, there is provided a kit comprising: (1) a pharmaceutical composition comprising: a) an engineered mammalian (such as human) immune cell comprising a heterologous nucleic acid encoding an immunomodulator, wherein the heterologous nucleic acid is operably linked to a promoter, wherein the engineered mammalian immune cell further expresses a CAR or TCR; and b) a pharmaceutically acceptable excipient; and (2) an instruction for using the pharmaceutical composition. In some embodiments, the engineered mammalian immune cell is a PBMC, a T cell, or an NK cell. In some embodiments, the promoter is inducible, such as by the intracellular signaling domain of the CAR or TCR In some embodiments, the promoter is a T cell activation dependent promoter, such as an IL-2 promoter, an NFAT promoter, or an NFκB promoter. In some embodiments, the immunomodulator is an immune checkpoint inhibitor (such as an inhibitor of CTLA-4, or an inhibitor of PD-1). In some embodiments, the immunomodulator is an immunoactivator. In some embodiments, the immunomodulator is a secreted protein. In some embodiments, the immunomodulator is an antibody (such as full-length antibody, scFv, single-domain antibody, heavy chain-only antibody, or Fab). In some embodiments, the engineered mammalian cell further comprises a second heterologous nucleic acid encoding a therapeutic protein (such as a second immunomodulator, for example, an immunoactivator; or a therapeutic protein that is not an immunomodulator, for example, chemotherapeutic antibody, e.g., an anti-HER2 antibody). In some embodiments, the immunoactivator is selected from the group consisting of IL-2, IL-7, IL-15, IL-21, IL-12, CCR4, CCR2b, Heparanase, CD137L, LEM, and Bcl-2. In some embodiments, the CAR is encoded by a third heterologous nucleic acid operably linked to a second promoter. In some embodiment, the second promoter is a constitutive promoter. In some embodiments, the second promoter is inducible, for example, by an inducing condition selected from inducer (such as small molecule, for example, tetracycline, or doxycycline), irradiation, temperature, redox state, tumor environment, and the activation state of the engineered mammalian immune cell. In some embodiments, the CAR or TCR targets a tumor antigen, such as EGFR, e.g., EGFRvIII, BCMA, or NY-ESO-1. In some embodiments, the kit further comprises an inducer.

In some embodiments, there is provided a kit comprising: (I) a pharmaceutical composition comprising: a) an engineered mammalian (such as human) immune cell comprising a heterologous nucleic acid encoding an immune checkpoint inhibitor and/or an immunoactivator, and a CAR, wherein the heterologous nucleic acid is operably linked to a promoter; and b) a pharmaceutically acceptable excipient; and (2) an instruction for using the pharmaceutical composition. In some embodiments, the heterologous nucleic acid encodes the immune checkpoint inhibitor, the immunoactivator, and the CAR. In some embodiments, the heterologous nucleic acid encodes at least two immunoactivators. In some embodiments, the engineered mammalian immune cell is a PBMC, a T cell, or an NK cell. In some embodiments, the promoter is a constitutive promoter, such as hEF1α promoter. In some embodiments, the promoter is inducible. In some embodiments, the promoter is a T cell activation dependent promoter, such as an IL-2 promoter, an NEAT promoter, or an NFκB promoter. In some embodiments, the immune checkpoint inhibitor is an inhibitor of an immune checkpoint molecule selected from the group consisting of PD-1, PD-L1, PD-L2, CTLA-4, BLTA, TIM-3, or LAG-3. In some embodiments, the immune checkpoint inhibitor is an antibody (such as full-length antibody, scFv, single-domain antibody, heavy chain-only antibody, or Fab). In some embodiments, the immunoactivator is selected from the group consisting of IL-2, IL-7, IL-15, IL-21, IL-12, CCR4, CCR2b, Heparanase, CD137L, LEM, and Bcl-2. In some embodiments, the CAR targets a tumor antigen, such as EGFR, e.g., EGFRvIII, BCMA, or NY-ESO-1. In some embodiments, the CAR comprises an intracellular signaling domain with an abolished or attenuated immune effector function. In some embodiments, the CAR is a truncated CAR In some embodiments, the CAR does not comprise a primary intracellular signaling domain (such as CD3ζ). In some embodiments, the CAR comprises a nonfunctional or attenuated primary intracellular signaling domain (such as a mutant CD3ζ).

In some embodiments, there is provided a kit comprising: (1) a pharmaceutical composition comprising: a) an engineered mammalian cell comprising a heterologous nucleic acid encoding an immuno modulator, wherein the heterologous nucleic acid is operably linked to a promoter; b) a second mammalian immune cell expressing a chimeric antigen receptor (CAR) or a recombinant T cell receptor (TCR); and c) a pharmaceutically acceptable excipient; and (2) an instruction for using the pharmaceutical composition. In some embodiments, the engineered mammalian cell is an immune cell (such as a PBMC, an NK cell, or a T cell). In some embodiments, the engineered mammalian c ell is a stem cell. In some embodiments, the promoter is inducible by an inducing condition selected from inducer (such as small molecule, for example, tetracycline, or doxycycline), irradiation, temperature, redox state, tumor environment, and the activation state of the engineered mammalian cell. In some embodiments, the immunomodulator is an immune checkpoint inhibitor (such as an inhibitor of CTLA-4, or an inhibitor of PD-1). In some embodiments, the immunomodulator is an immunoactivator. In some embodiments, the immunomodulator is a secreted protein. In some embodiments, the immunomodulator is an antibody (such as full-length antibody, scFv, single-domain antibody, heavy chain-only antibody, or Fab). In some embodiments, the engineered mammalian cell further expresses on its surface a targeting molecule recognizing a tumor antigen. In some embodiments, the engineered mammalian cell further comprises a second heterologous nucleic acid encoding a therapeutic protein (such as a second immuno modulator, for example, an immunoactivator; or a therapeutic protein that is not an immunomodulator, for example, chemotherapeutic antibody). In some embodiments; the immunoactivator is selected from the group consisting of IL-2, IL-7, IL-15, IL-21, IL-12, CCR4, CCR2b, Heparanase, CD137L, LEM, and Bcl-2. In some embodiments, the second mammalian immune cell is a PBMC, a T cell or an NK cell. In some embodiments, the CAR or TCR targets a tumor antigen, such as EGFR, e.g., EGFRvIII, BCMA, or NY-ESO-1. In some embodiments, the kit further comprises an inducer.

In some embodiments, there is provided a kit comprising: (1) a pharmaceutical composition comprising: a) an engineered mammalian cell comprising a heterologous nucleic acid encoding an immuno modulator, wherein the heterologous nucleic acid is operably linked to a promoter; and b) a pharmaceutically acceptable excipient; (2) a composition comprising a second mammalian immune cell expressing a CAR or a TCR; and (3) an instruction for using the pharmaceutical composition. In some embodiments, the engineered mammalian cell is an immune cell (such as a PBMC, an NK cell, or a T cell). In some embodiments, the engineered mammalian cell is a stem cell. In some embodiments, the promoter is inducible by an inducing condition selected from inducer (such as small molecule, for example, tetracycline, or doxycycline), irradiation, temperature, redo x state, tumor environment, and the activation state of the engineered mammalian cell. In some embodiments, the immunomodulator is an immune checkpoint inhibitor (such as an inhibitor of CTLA-4, or an inhibitor of PD-1). In some embodiments, the immunomodulator is an immunoactivator. In some embodiments, the immunomodulator is a secreted protein. In some embodiments, the immunomodulator is an antibody (such as full-length antibody, scFv, single-domain antibody, heavy chain-only antibody, or Fab). In some embodiments, the engineered mammalian cell further expresses on its surface a targeting molecule recognizing a tumor antigen. In some embodiments, the engineered mammalian cell further comprises a second heterologous nucleic acid encoding a therapeutic protein (such as a second immunomodulator, for example, an immunoactivator; or a therapeutic protein that is not an immunomodulator, for example, chemotherapeutic antibody). In some embodiments, the immunoactivator is selected from the group consisting of IL-2, IL-7, IL-15, IL-21, IL-12, CCR4, CCR2b, Heparanase, CD137L, LEM, and Bcl-2. In some embodiments, the second mammalian immune cell is a PBMC, a T cell or an NK cell. In some embodiments, the CAR or TCR targets a tumor antigen, such as EGFR, e.g., EGFRvIII, BCMA, or NY-ESO-1. In some embodiments, the pharmaceutical composition and the composition comprising the second mammalian immune cell are admixed prior to administration. In some embodiments, the kit further comprises an inducer.

The kits may contain one or more additional components, such as containers, reagents, culturing media, inducers, cytokines, buffers, antibodies, and the like to allow propagation or induction of the engineered mammalian cell and optionally the second mammalian immune cell. The kits may also contain a device for local administration (such as intratumoral injection) of the pharmaceutical composition and/or the composition comp rising the second mammalian immune cell to a tumor site.

The instructions relating to the use of the pharmaceutical composition and optionally the composition comprising the second mammalian immune cell generally include information as to dosage, dosing schedule, and route of administration for the intended treatment. In some embodiments, the instruction further includes information for inducing expression of the immunomodulator and/or other therapeutic proteins, for example, dosage, dosing schedule, and route of administration of the inducer. The containers may be unit doses, bulk packages (e.g., multi-dose packages) or sub-unit doses. In some embodiments, the total amount of the composition (such as pharmaceutical composition, composition comprising the second mammalian immune cell, and/or the inducer) is enough for a full dosage for a single local administration (such as intratumoral injection). In some embodiments, the total amount of the composition (such as pharmaceutical composition, composition comprising the second mammalian immune cell, and/or the inducer) is enough for asp lit dosage for a singe local administration (such as intratumoral injection) to one of a plurality of tumor sites. In some embodiments, the total amount of the composition (such as pharmaceutical composition, composition comprising the second mammalian immune cell, and/or the inducer) is enough for multiple local administrations, including a combination of a single local administration (such as intratumoral injection) into one tumor site and multiple sp lit-dosage administrations at multiple tumor sites.

For example, kits may be provided that contain sufficient dosages of the pharmaceutical composition as disclosed herein to provide effective treatment of an individual for an extended period, such as any of a week, 8 days, 9 days, 10 days, 11 days, 12 day s, 13 days, 2 weeks, 3 weeks, 4 weeks, 6 weeks, 8 weeks, 3 months, 4 months, 5 months, 7 months, 8 months, 9 months, or more. Kits may also include multiple unit doses of the pharmaceutical composition and instructions for use, packaged in quantities sufficient for storage and use in pharmacies, for example, hospital pharmacies and compounding pharmacies.

The kits of the invention are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder described herein, and may have a sterile access port (for example the container may be an intravenous solution bag or a vial having a stopper pierceable by a hypodermic injection needle). The label or package insert indicates that the composition is used for treating the particular condition in an individual. The label or package insert will further comprise instructions for administering the composition to the individual. Articles of manufacture and kits comprising combination therapies described herein are also contemplated.

Package insert refers to instructions customarily included in commercial packages of therapeutic products that contain information about the indications, usage, dosage, administration, contraindications and/or warnings concerning the use of such therapeutic products. In some embodiments, the package insert indicates that the composition is used for treating a solid tumor (such as glioblastoma).

Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Examples

The examples below are intended to be purely exemplary of the invention and should therefore not be considered to limit the invention in any way. The following examples and detailed description are offered by way of illustration and not by way of limitation.

### Example 1: Expression of Functional Antibodies in Primary T Cells and Other Mammalian Cells

The self-inactivating lentiviral vectors carrying an antibody gene driven by a constitutive promoter hEF1α, a doxycycline inducible promoter (such as TETON®), an NFAT-dependent inducible promoter, or a heat inducible promoter (such as human heat shock protein 70 promoter, HSP70p) were designed and prepared. Each antibody gene can express an antibody specifically against a unique antigen selected from PD-1, CTLA-4, and any other targets of interest. Primary human peripheral blood mononuclear cells (PBMC) were prepared by density gradient centrifugation of peripheral blood from healthy donors. Human primary T cells were purified from PBMCs using magnetic bead isolation, and pre-activated. The pre-activated T cells were then transduced with the lentiviral vectors and expanded ex vivo for a few days. Alternatively, other host cells, such as 293-6E cells, Jurkat cells, mesenchymal stem cells, purified human B cells, and other PBMC cells can be transduced with the lentiviral vectors, and used to express the antibodies. The secretion of antibody was detected using homogenous time-resolved fluorescence (HTFR) technology. Alternatively, the secreted antibodies can be detected by recombinant antigen-tag protein via enzyme linked immunosorbent assay (ELISA). The bioactivity of the secreted antibodies were assessed using an in vitro reporter assay.

The materials and methods used in these experiments are described as below.

### Preparation of Self-Inactivating Lentiviral Vectors

The lentivirus packaging plasmid mixture including pMDLg/pRRE (Addgene#12251), pRSV-Rev (Addgene#12253), and pMD2.G (Addgene#12259) was pre-mixed with an antibody-expression plasmid pLLV-promoter-anti-PD-1 vector (i.e., pLLV-hEF1α-anti-PD-1, pLLV-TetOn-anti-PD-1, pLLV-NFAT-anti-PD-1, or pLLV-HSP70p-anti-PD-1), or pLLV-promoter-anti-CTLA-4 vector (i.e., pLLV-hEF1α-anti-CTLA-4, pLLV-TetOn-anti-CTLA-4, pLLV-NFAT-anti-CTLA-4, or pLLV-HSP70p-anti-CTLA-4) at a pre-optimized ratio with poly etherimide (PEI), then mixed properly and incubated at room temperature for 5 minutes. The transfection mix was then added dropwise to the HEK293 cells and mixed gently. Afterwards, cells were incubated overnight in a 37° C. and 5% CO₂ cell incubator. The supernatants were collected after centrifugation at 4° C., 500 g for 10 min.

After the supernatants were filtered through a 0.45 μm PES filter, the virus supernatants were concentrated with 20% sucrose gradient ultracentrifugation. After centrifugation, the supernatants were carefully discarded and the virus pellets were rinsed cautiously with pre-chilled DPBS. The concentration of virus was then measured. Virus was aliquoted properly, then stored at −80° C. immediately. The virus titer was determined by p24 based on HT RF kit developed by GenScript.

### PBMC Preparation

Leukocytes were collected, and cell concentration was adjusted to 5×10⁶ cells/mL in R10 medium. Leukocytes were then mixed with 0.9% NaCl solution at 1:1 (v/v) ratio. 3 mL lymphoprep medium was added to a 15 mL centrifuge tube, and on top of lymphoprep was slowly layered 6 mL of diluted lymphocyte mix. The lymphocyte mix was centrifuged at 800 g for 30 minutes without brakes at 20° C. Lymphocyte buffy coat was then collected with a 200 μL pipette. The harvested fraction was diluted with at least 6 folds of 0.9% NaCl or R10 to reduce the density of the solution. The harvested fraction was then centrifuged at 250 g for 10 minutes at 20° C. The supernatant was aspirated completely, and 10 mL of R10 was added to the cell pellet. The mixture was further centrifuged at 250 g for 10 minutes at 20° C. The supernatant was then aspirated. 2 mL 37° C. pre-warmed R10 with 100115/mL IL-2 was added to the cell pellet, and the cell pellet was resuspended softy. The number of cells was then counted, and the PBMC sample was ready for later experiments.

### T Cell Purification

Human T cells were purified from PBMCs using Miltenyi Pan T cell isolation kit (Cat#130-096-535), following the protocol provided by the manufacturer as below. Cell number was first determined. The cell suspension was centrifuged at 300 g for 10 minutes. Supernatant was then aspirated completely, and cell pellets were resuspended in 40 μL buffer per 10⁷ total cells. 10 μL of Pan T Cell Biotin-Antibody Cocktail was added per 10⁷ trial cells, mixed thoroughly and incubated for about 5 minutes in the refrigerator (2-8° C.). 30 μL of buffer was then added per 10⁷ cells. 20 μL of Pan T Cell MicroBead Cocktail was added per 10⁷ cells. The mixture was mixed wetland incubated for an additional 10 minutes in the refrigerator (2˜8° C.). A minimum of 500 μL was required for magnetic separation. LS column was placed in the magnetic field of a suitable MACS Separator. The column was prepared by rinsing with 3 mL of buffer. Cell suspension was then applied onto the column, and flow-through containing unlabeled cells was collected, representing the enriched T cell fractions. T cells were then collected by washing column with 3 mL of buffer, collecting unlabeled cells that pass through, which represent the enriched T cells, and combining with the flow-through from previous step. T cells were then resuspended in R10+100 IU/mL IL-2. The primary T cells were then pre-activated with human T Cell Activation/Expansion Kit (Miltenyi #130-091-441) for 3 days prior to transduction.

### B Cell Purification

Primary human B cells were also prepared with magnetic beads isolation strategies. PBMCs were prepared by density gradient centrifugation as described above. Human B cells were purified from PBMCs using Miltenyi Human B cell isolation kit (Cat#130-091-151), following a similar protocol of preparing human T cells as described above. The isolated human B cells were cultured ex vivo in RPM11640 media supplemented with recombinant CD40 and IL4 proteins (Martina et al. PLoS ONE 3(1): e1464 (2008)).

### Natural Killer (NK) Cell Purification

Primary human NK cells were prepared with magnetic beads isolation strategies. PBMCs were prepared by density gradient centrifugation as described above. Human NK cells were purified from PBMCs using Miltenyi Human NK cell isolation kit (Cat #130-092-657), following a similar protocol of preparing human T cells as described above. The isolated human NK cells were cultured ex vivo in α-MEM media supplemented with 200 IU/mL recombinant IL-2 proteins.

### Preparation of Mesenchymal Stem Cells

Bone marrow from human donor is collected by aspiration from iliac crest under local anesthesia, and mononuclear cells are subsequently isolated by Ficoll separation techniques. Cells are then washed and resuspended in MSC culture medium (Dulbecco's modified Eagle's medium-low glucose/penicillin/streptomycin/10% fetal calf serum), plated in tissue culture flasks and incubated at 37° C. and 5% CO₂. MSCs are expanded according to the standardized LUMC protocol for expansion of MSCs. Twice a week, cultures are microscopically examined and medium is refreshed. Cells are trypsinized when >70% confluence is readied and MSC half products (passage 1) of various sizes are cryopreserved with 10% dimethyl sulfoxide.

### Expression of Antibody Gene in Host Cells

Host cells including primary human T cells, purified human B cells, and purified human NK cells were transduced with serially diluted virus stock in the presence of 7 mg/mL polybrene by centrifugation at 1200 g, 32° C. for 1.5 h. The transfected cells are then transferred to the cell culture incubator for transgene expression under suitable induction conditions. Specifically, host cells transduced with pLLV-hEF1α-anti-PD-1 or pLLV-hEF1α-anti-CTLA-4 were incubated without an inducer or induction condition for 48 hours. Host cells transduced with pLLV-TetOn-anti-PD-1 or pLLV-TetOn-anti-CTLA-4 were incubated with doxycycline at various concentrations (0˜12 μg/mL) for 48 hours. Host cells transduced with pLLV-NFAT-anti-PD-1 or pLLV-NFAT-anti-CTLA-4 were incubated with a T cell activation composition PM ALPHA-P at various concentrations (0-50 ng/mL PMA·1000 ng/mL PHA-P) for 48 hours. Host cells transduced with pLLV-HSP70p-anti-PD-1 or pLLV-HSP70p-anti-CTLA-4 were heat-shocked at 37° C., 39° C., 41° C., 43° C. or 44° C. for 20 min in a temperature controlled water bath immediately after transduction. After heat shock, the cells were seeded back to 6 well plates and continued to grow at 37° C./5% CO₂ cell culture incubator for 3 days. For other constructs having a temperature-inducible promoter, the host cells can be induced at an optimal temperature (such as 37° C. to 45° C.) for a period of time (e.g., 10 min, 20 min, or 30 min, etc.) after 24-72 hours of incubation.

After induction of transgene expression, secreted antibodies from each batch of host cells can be detected using a variety of methods, including, for example, Homogenous time-resolved fluorescence (HTRF, also known as Time resolved-fluorescence resonance energy transfer, or TR-FRET) or ELISA. For ELISA, briefly, wells of the MAXI-SORP® ELISA plate (Nunc, cat#44-2404-21) are pre-coated with goat anti-human IgG-UNLB. After blocking and washing the supernatant from transduced cells are added to the plate serially. After washing goat anti-human Kappa-HRP is added to the plate, then HRP substrate DAB is added following standard ELI SA procedures. The plate is then read on a micro-plate reader such as FLEXSTATION™ 3.

Here, secreted anti-PD-1 and anti-CTLA-4 antibodies from each batch of host cells were detected by Homogenous time-resolved fluorescence (HTRF) technology using LANPOWER™ Human Fc Detection kit (GenScript # L00656-1000). The kit can be used for detecting human Fc-tagged proteins or human IgG in a sample. The kit is a competitive immunoassay, including a polyclonal antibody (Fc specific) labeled with Europium (LANPOWER™ Eu), and human IgG labeled with GS665 dye. When the Eu-labeled polyclonal antibody binds to the Fc region of the GS5 labeled human IgG, FRET occurs. If samples containing human IgG or human Fc-tagged protein are added, the FRET signal will be reduced. The detected FRET signal is inversely correlated with the concentration of human IgG or human Fc-tagged protein in the added samples. Briefly, human IgG-GS665, anti-human Fc antibody-Eu, and the antibody sample or controls were mixed in an assay plate and incubated for 1.5 h, The plates were read on HTRF compatible instruments (PHERSTAR™ plus microplate reader, Ex: 320-340 nm, Em:620 nm and 665n m).

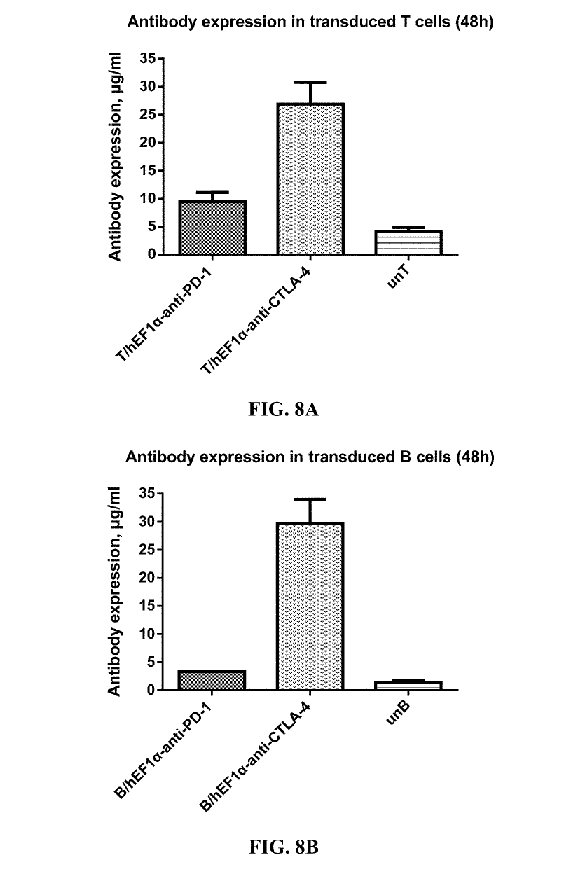

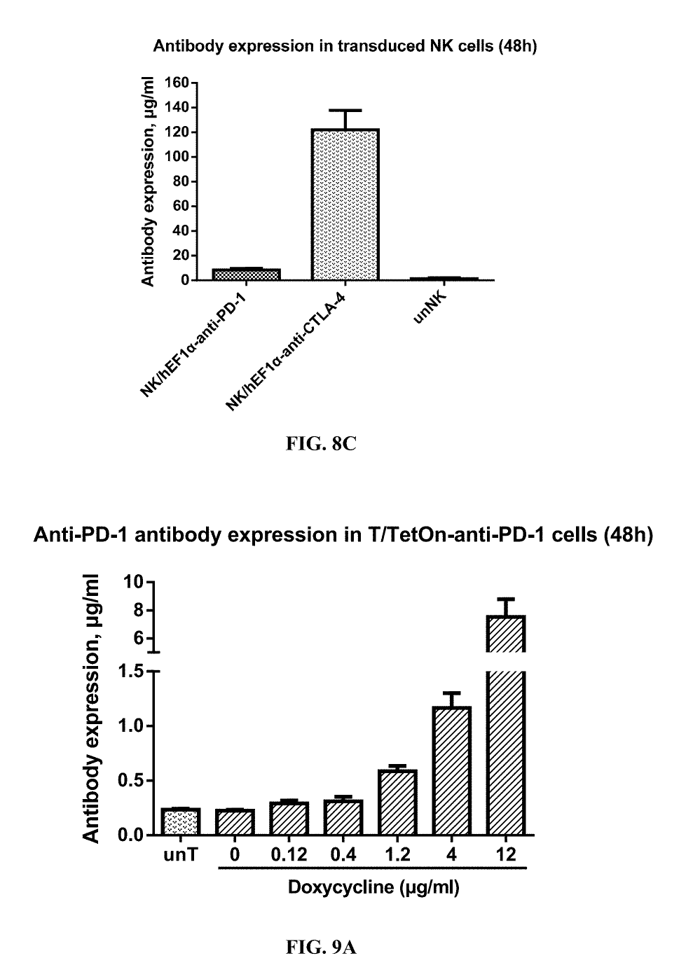

When transduced with lentiviral vector pLLV-hEF1α-anti-PD-1 (or pLLV-hEF1α-anti-CTLA-4), host cells (primary human T cells, purified human B cells and purified human NK cells) constitutively secreted anti-PD-1 antibody (or anti-CTLA-4 antibody), which were successfully detected by HTRF at 48 h post transduction (FIGS. 8A-8C). Anti-PD-1 antibody was secreted at a level of 9.45±1.17 mg/mL, 3.31±0.01 μg/mL and 8.5110.83 μg/mL respectively from transduced T cells, B cells and NK cells. The expression level of anti-CTLA-4 antibody was much higher than that of the anti-PD-1 antibody in the same type of host cells. The concentrations of anti-CTLA-4 antibody detected in the supernatant of transduced T cells, B cells and NK cells were 26.8912.74 ng/mL, 29.66±3.06 ng/mL and 122.12±11.09 μg/mL respectively.

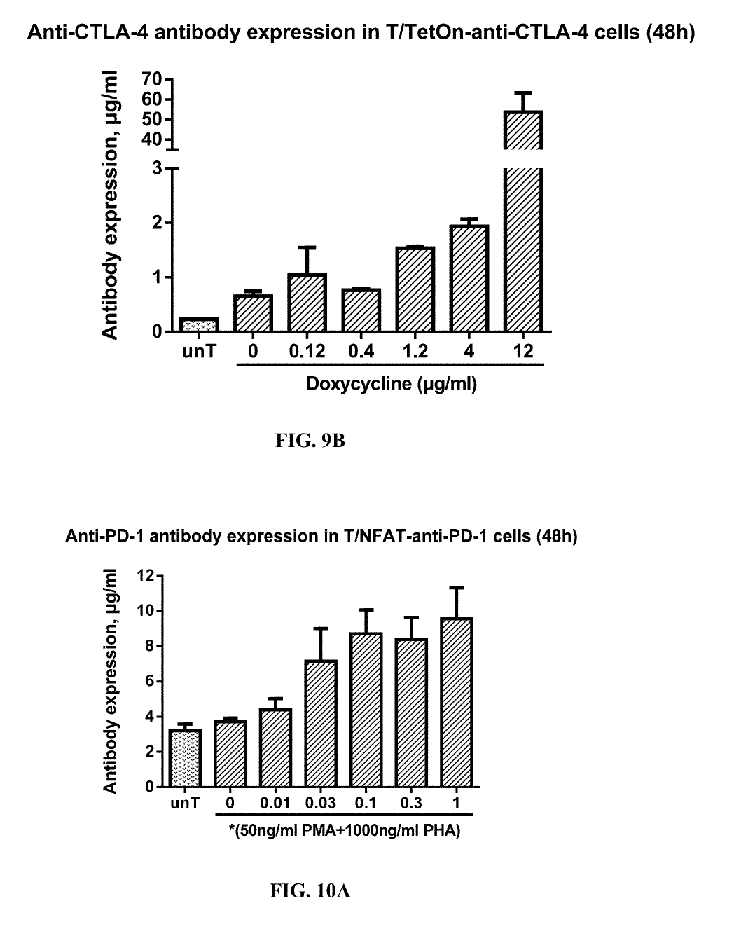

When primary human T cells were transduced with lentivirus vector pLLV-TetOn-anti-PD-1 (or pLLV-TetOn-anti-CTLA-4), anti-PD-1 antibody (or anti-CTLA-4 antibody) was secreted in a dose-dependent manner with respect to the inducer doxycycline (FIGS. 9A-9B). After a 48 h induction period with 12 μg/mL doxycycline, 7.5310.89 ng/mL of anti-PD-1 antibody and 53.67±6.74 μg/mL of anti-CTLA-4 antibody were detected in the supernatant of corresponding transduced T cells respectively. Without doxycycline, spontaneous expression of both anti-PD-1 and anti-CTLA-4 antibodies was much lower than corresponding transduced cells treated with doxycycline.

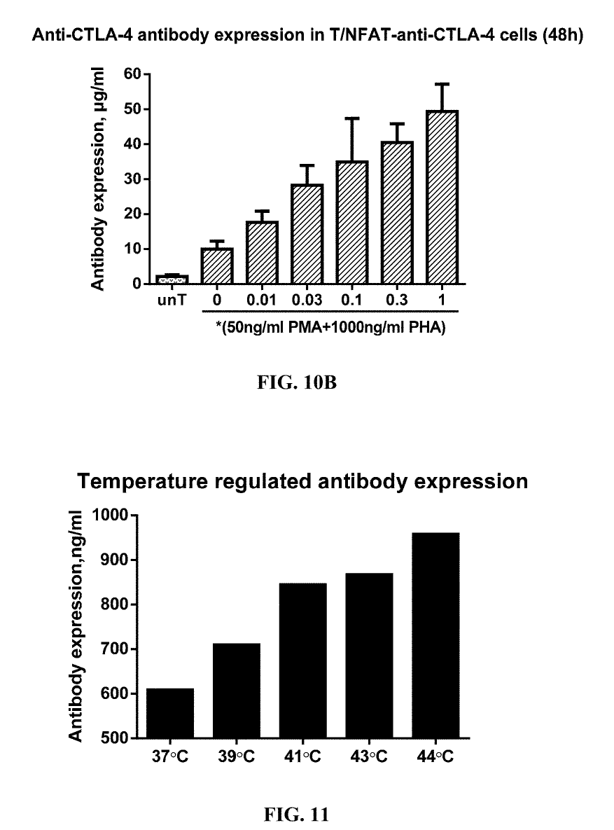

When primary human T cells were transduced with lentivirus vector pLLV-NFAT-anti-PD-1 (or pLLV-NFAT-anti-CTLA-4), anti-PD-1 antibody (or anti-CTLA-4 antibody) was secreted in a dose-dependent manner with respect to the T cell activators such as PM A/PHA-P (FIGS. 10A-10B). Similar to the above results, the anti-CTLA-4 antibody was secreted at much higher levels than the anti-PD-1 antibody from transduced primary human T cells. After a 48 h induction period with 50 ng/mL PMA and 1000 ng/mL PHA-P, the anti-M-1 and anti-CTLA-4 antibody secretion reached 9.57±1.24 μg/mL and 49.39±5.53 μg/mL, respectively. Without the presence of inducers PMA/PHA-P, spontaneous antibody expression was low compared to corresponding host cells treated with PMA/PHA-P.

As shown in FIG. 11, when primary T cells were transduced with lentivirus vector pLLV-HSP60p-anti-PD-1, and heat shocked at an elevated temperature, anti-PD-1 antibody was secreted at a significantly increased level (709.5 ng′mL at 39° C., 844.2 ng′mL at 41° C., 866.8 ng′mL at 43° C., 957.8 ng/mL at 44° C.) as compared to corresponding transduced host cells incubated at physiological temperature (608.4 ng/mL at 37° C.).

### Example 2: Functional Assay of In Vitro Secreted Antibodies by Engineered Host Cells

### Development of Reporter Cell Lines

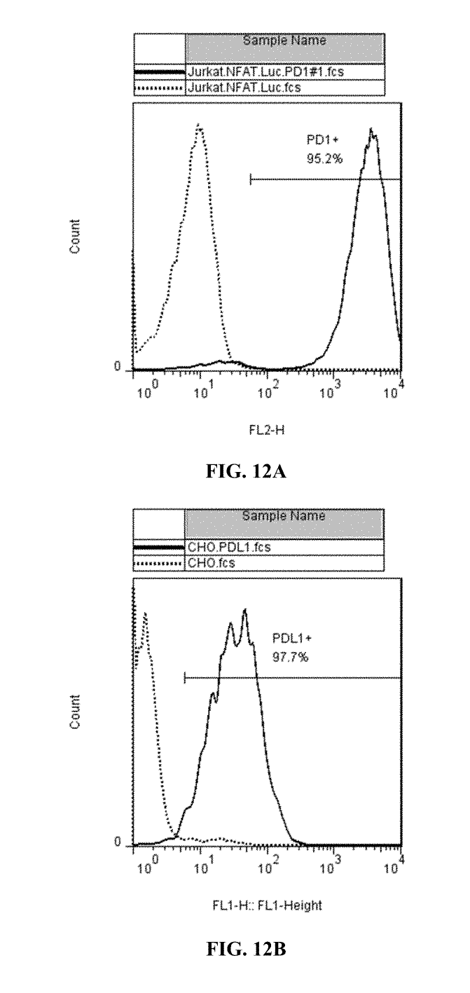

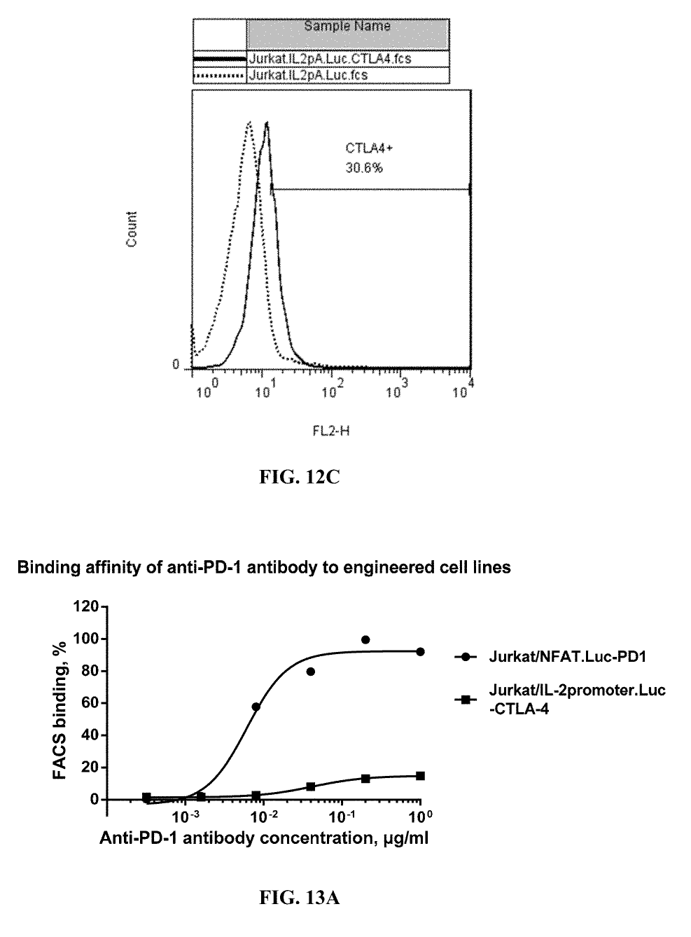

A reporter cell line stably expressing PD-1 was established. Briefly, a lentiviral vector was modified using pLVX-Puro (Clontech#632164) by rep lacing the original promoter with human elongation factor 1α promoter (hEF1α) and replacing the p uromycin resistance gene with a T2A linked G418 resistance gene with EcoRI and XbaI by GenScript. The vector was named pLLV-hEF1α-T2A-G418R and several new restriction sites MluI, HpaI and BamHI were included in the vector during molecular cloning. Human PD-1 gene (N CBI reference sequence ID: NM_005018.2) sequence was cloned into the pLLV-hEF1α-T2A-G418R vector via EcoRI/H paI to provide pLLV-hEF1α-PD-1-T2A-G418R, which was further subject to the lentivirus packaging procedure as described in the examples above. The host cell line Jurkat/NFAT.Luc (puromycin resistant), previously established in house was transduced with lentivirus carrying PD-1 gale by centrifugation at 1200 g, 32° C. for 1.5 h in the presence of 7 μg/mL polybrene. The transduced cells were then transferred to the cell culture incubator for transgene expression under suitable conditions. Positive cells were selected with neomycin (G418), and single clones were selected by limiting dilution. The best clones were picked by FACS using anti-PD-1 antibodies. As shown in FIG. 12A, 95.2% of an exemplary positive clone named Jurkat/NFAT.Luc-PD-1 (also known as Jurkat.NFAT.Luc.PD1) expressed PD-1 protein.

A reporter cell line stably expressing PD-L1 was also developed. Briefly, human PD-L1 gene (NCBI reference sequence ID: NM 014143.3) sequence was cloned into the pLLV-hEF1α-T2A-G418R vector via EcoRI/HpaI to provide the vector pLLV-hEF1α-PD-L1-T2A-G418R, which was further subject to the lentivirus packaging procedure as described in the examples above. The host cell line CHO was then transduced with the lentivirus carrying PD-L1 gene, and selected with G418 as described above. CHO/PD-L1 stable cells were selected by FACS by staining with 1.25 μg/ml PD-1-Fc fusion protein, 3 wash with DPBS, followed by staining with 2 μg/mL FITC labeled anti human Fc. As shown in FIG. 12B, 95.2% of an exemplary positive clone named CHO/PD-L1 (also known as CHOPDL1) expressed PD-L1 protein.

A reporter cell line stably expressing CTLA-4 was also established. Human CTLA-4 gene (NCBI reference sequence ID: NM_005214.4) sequence was cloned into the pLLV-hEF1α-T2A-G418R vector via EcoRI/HpaI to provide the vector pLLV-hEF1α-CTLA4-T2A-6418R, which was further subject to the lentivirus packaging procedure as described in the examples above. The lentivirus carrying human CTLA-4 gene was introduced into Jurkat.IL-2 promoter A. Luc stable cells, which were previously made in-house. Positive cells were selected with neomycin (G418) and single clones were selected by Uniting dilution. Best clones were picked by FACS using anti-CTLA-4 antibodies. As shown in FIG. 12C, 30.6% of an exemplary clone named Jurkat/IL-2 promoter.Luc-CTLA-4 (also known as Jurkat.IL2 pA.Luc.CTLA4) expressed CTLA-4 protein.

### Direct FACS Binding Assay

The binding affinity of secreted anti-PD-1 antibodies was determined by binding to PD-1 protein expressed on the stable cell line Jurkat/NFAT.Luc-PD-1. Briefly, 5×10⁵ Jurkat/NFAT.Luc-PD-1 cells were incubated with serially diluted supernatants containing secreted anti-PD-1 antibodies (0, 0.1, 0.3, 1, 3, 10 μg/mL) from the engineered host cells of Example 1 for 2 hours at room temperature. After a few cycles of cell washing, fluorophore-labeled secondary antibodies against human IgG were added to detect anti-PD-1 antibodies bound to cells by FACS. Jurkat/CTLA-4 cells were used as negative controls.

The binding affinity of secreted anti-CTLA-4 antibodies was determined by binding to CTLA-4 protein expressed on the stable cell line Jurkat/IL-2 promoter.Luc-CTLA-4 developed in-house. 5×10⁵ Jurkat/IL-2 promoter.Luc-CTLA-4 cells were incubated with serially diluted supernatants containing secreted anti-CTLA-4 antibodies from the engineered host cells of Example 1 for 2 hours at room temperature. After a few cycles of cell washing, fluorophore labeled secondary antibodies against human IgG were added to detect anti-CTLA-4 antibodies bound to cells by FACS. Jurkat/PD-1 cells were used as negative controls.

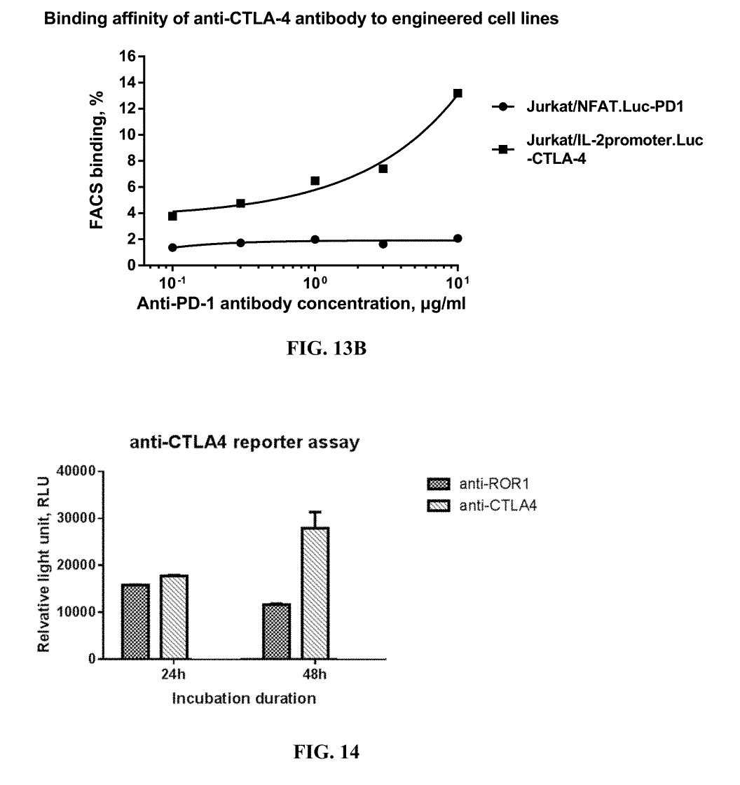

As shown in FIG. 13A, secreted anti-PD-1 antibody from the engineered host cells bound to the Jurkat/NFAT.Luc-PD-1 stable cells in a dose-dependent manner, and 99.5% binding was achieved at the concentration of 0.2 μg/mL. In contrast, anti-PD-1 antibody did not bind to Jurkat/IL-2 promoter.Luc-CTLA-4 cells. As shown in FIG. 13B, secreted anti-CTLA-4 antibody from the engineered host cells bound to the Jurkat/IL-2 promoter.Luc-CTLA-4 stable cells in a dose-dependent manner (3.77% at 0.1 μg/mL, 6.49% at 1 μg/mL, 13.20% at 10 μg/mL), but not to Jurkat/NFAT.Luc-PD-1 cells (<2%).

### Functional Activities of Antibodies Expressed by Transduced Stable Cell Lines

In order to assess the effect of anti-PD-1 antibody on restoring T cell activation, 1˜5×10⁵ Jurkat/NFAT.Luc-PD-1 reporter cells are incubated with CHO/PD-L1 cells at different E/T ratios (e.g. 1:1, 10:1, 20:1, 1:10, 1:20) in the presence of secreted anti-PD-1 antibody (such as pembrolizumab, for example having the sequence with Accession Number DB09037 on world wide web.Drugbank.ca) from the engineered host cells of Example 1 for a period of time (e.g. 4 h to 72 h). Lentiviral vectors carrying an irrelevant gene are transduced to the same cell lire side by side as a negative control.

1×10⁵˜5×10⁵ Jurkat/IL-2 promoter.Luc-CTLA-4 reporter cells are incubated with antigen presenting cells expressing CD80/CD86 (such as Raji or U87M G, etc.) at different E/T ratios (e.g., 1:1, 10:1, 20:1, 1:10, 1:20, etc.) in the presence of secreted anti-CTLA-4 antibody from the engineered host cells of Example 1 for a period of time (e.g., 4 h to 72 h). Lentiviral vectors carrying an irrelevant gene are transduced to the same cell line side by side as a negative control.

Non-antigen specific T cell activators, such as anti-CD3/CD-28 beads or PMA/PHA-P, are added to activate Jurkat/NFAT.Luc-PD-1 or Jurkat/IL-2 promoter.Luc-CTLA-4 reporter cells. After incubation, ONE-GLO™ luciferase assay reagents are added to the co-cultured cells. The luciferase activity from assay wells measured by relative light unit (RLU) is presented as the activation degree of each reporter cells.

Secreted anti-PD-1 antibodies from the engineered host cells may block PD-1/PD-L1 interactions, thus restoring T cell activation as suggested by the increase of RLU compared to the negative control expressing human IgG antibody. And such effect may be dose dependent on the concentration of anti-PD-1 antibodies.

Secreted anti-CTLA-4 antibodies from the engineered host cells may block CTLA-4/CD80-CD86 interaction, thus restoring T cell activation as suggested by the increase of RLU compared to the negative control expressing human IgG antibody. And such effect may be dose dependent on the concentration of anti-CTLA-4 antibodies.

### Reporter Assay of CTLA-4 Blockade by Transduced Stable Cell Lines

Human lymphoma cell line Raji (ATCC, #CCL-86) were cultured in RPMI1640 medium supplemented with 10% FBS according to the manufacturer's instructions. Raji cells have been shown to express CTLA-4 ligands CD80 and CD86 at high levels (International Immunology 10(4):499-506. May 1998). An anti-CTLA-4 antibody was obtained by transducing human HEK293T cells with lentiviral vectors carrying Ipilimumab full IgG coding sequence (world wide web.DrugBank.ca, Accession number: Ipilimumab DB06186). The transduced cells were cultured under suitable conditions for secretion of anti-CTLA4, and supernatants were harvested for cell based assays. 1×10⁵ Jurkat/IL-2 promoter.Luc.CTLA-4 cells were seeded into a 96-well assay plate (Corning#3610), followed by addition of anti-ROR1, or anti-CTLA-4 at a final concentration of 20 μg/mL, and incubated for 1 hour in a 37° C. cell culture incubator. Subsequently, 3.2×10⁵ Raji cells were added to each well and the co-culture assay was continued for another 24 or 48 hours. Upon completion of co-culture, luciferase activity in each well was determined using the ONE-GLO™ Luciferase activity assay kit (Promega #E6110) per the manufacturer's manual. The plates were read on a PHER STAR™ Plus microplate reader. As shown in FIG. 14, when the co-culture assays were carried out for 48 h, anti-CTLA-4 (Ipilimumab) showed around 2.4 higher RLU signal than unrelated antibody (anti-ROR1) (27914.00±3431.00 RLU versus 11585.00±303.00, mean±s.e.). This data suggested that CTLA-4 blockade could potent restore the IL-2 promoter driven luciferase gene expression in the CTLA-4 reporter assays.

### Functional Activities of Antibodies Expressed by Engineered Primary Human T Cells In Vitro

The PD-L1 gene and one target gene (EGFRvIII), as well as luciferase gene, were introduced into human glioblastoma tumor cell line (U87MG) using lentiviral vectors. Briefly, human PD-L1 gene (N CBI reference sequence ID: NM_014143.3) sequence was cloned into the pLLV-hEF1α-T2A-G418R vector via EcoRI/HpaI to provide the vector pLLV-hEF1α-PD-L1-T2A-G418R, which was further subject to the lentivirus packaging procedure as described above. Human epidermal growth factor receptor variant III (EGFR viii) nucleotide sequence was obtained by deleting exons 2-7 from the wild type EGFR (NM_005228), resulting in an in-frame deletion of 801 base pairs of the coding sequence and the generation of a novel glycine residue at the fusion junction (Endocrine-Related Cancer (2001)883-96). The EGFR viii nucleotide sequence was cloned into the pLVX-Puro vector with XhoI/XbaI restriction enzymes to provide the pLVX-EGFRvIII vector, which was further subjected to lentivirus packaging as described above. Positive clones were screened with G418 and Puromycin and single clones were obtained by limiting dilution.

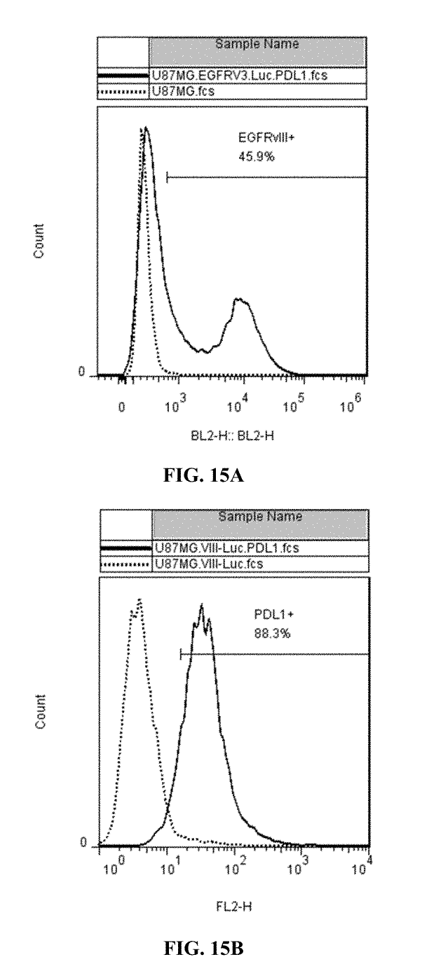

EGFRvIII expression was validated by binding the cells to Cetuximab, followed by 3×1 mL DBPS wash, staining with anti-human IgG detection antibody, and FACS analysis on an ATTUNENXT™ flow cytometer (Thermofisher). As shown in FIG. 15A, EGFRvIII transgene expression was detected on the cells (45.9% expression). PD-L1 expression was validated by staining cells with PE labeled anti-PD-L1 (Biolegend #329702) followed by FACS on FACSCALIBUR™ (BD Biosciences). As shown in FIG. 15B, PD-L1 expression was detected on 88.3% of the cells. Luciferase gene expression was validated by ONE-GLO™ luciferase assay kit (Promega) and read on PHERSTAR™ plus microplate reader (BMG Labtech). An exemplary optimal clone named U87MG/VIII-Luc-PD-L1 (also known as U87MG.EGFRV3.LucPDL1 or U87MG.VIII-Luc.PDL1) showed a 98.89 fold increase of luciferase activity as demonstrated by relative light unit signal compared to untransduced cells.

Similarly, the CTLA-4 ligand gene (CD80/CD86) and one target of our interest, as well as luciferase gene, are introduced into human tumor cell line (U87MG) using lentiviral vectors. One of the optimal clones is named as U87M G/VIII-Luc-CD80/CD86.

Primary T cells transduced with lentiviral vectors carrying anti-PD-1 gene (i.e., pLLV-hEF1α-anti-PD-1, pLLV-TetOn-anti-PD-1, pLLV-NFAT-anti-PD-1, or pLLV-HSP70p-anti-PD-1) are co-cultured with U87MG/VIII-Luc-PD-L1 cells over-expressing PD-L1 at different E/T ratios (e.g. 1:1, 10:1, 20:1, 1:10, 1:20) for a period of time (e.g. 4 h to 72 h) under suitable induction conditions. Primary human T cells transduced with lentiviral vectors carrying anti-CTLA-4 gene (i.e., pLLV-hEF1α-anti-CTLA-4, pLLV-TetOn-anti-CTLA-4, pLLV-NFAT-CTLA-4, or pLLV-HSP70p-CTLA-4) are co-cultured with U87MG/VIII-Luc-CD80/CD86 cells over-expressing CTLA-4 ligand under various induction conditions. Cytotoxic efficacy of antibody-secreting primary human T cells on tumor cells is monitored by remaining luciferase activity.

Primary human T cells transduced with an irrelevant gene are included in the assay side by side as a negative control. In the same assay format, IL-2 secretion in the co-culture assay is assayed using HTRF kit. In the same assay format, INF-gamma secretion in the co-culture assay is assayed using HTRF kit.

The secreted anti-PD-1 antibodies by the engineered primary T cells may block PD-1/PD-LI interaction, thus restoring T cell activation and cytotoxicity as illustrated by remaining luciferase activity in the well, and by increased secretion of IL-2 or IFN-gamma as compared to the negative control. Such effect may be dose dependent on the Effector/Target cell ratio.

The secreted anti-CTLA-4 antibodies by the engineered primary T cells may block CTLA-4/CD80-CD86 interaction, thus restoring T cell activation and cytotoxicity as illustrated by remaining luciferase activity in the well, and by increased secretion of IL-2 or IFN-gamma as compared to the negative control. Such effect may be dose dependent on the Effector/Target cell ratio.

### Example 3: Anti-PD-1 Antibody Expressed by Engineered Primary Human T Cells Augments CART Cytotoxicity Against Tumor Cells In Vitro

An anti-EGFRvIII-CAR construct (GSI026) was designed and introduced to lentiviral vectors. The anti-EGFRvIII-CAR gene comprises: a full-length anti-EGFRvIII CAR, including from the N-terminus to the C-terminus, CD8α signal peptide, humanized anti-EGFRvIII scFv, CD8α hinge and transmembrane (TM) region, CD137 cytoplasmic domain (CD137 cyto), and CD3ζ. See Chinese Patent Application No. CN 201611039855.0. The following experimental groups of T cells were prepared: (1) T/GSI026: engineered human primary T cells transduced with a lentiviral vector carrying an anti-EGFRvIII-CAR gene (GSI026); (2) T/anti-PD-1: engineered human primary T cells transduced with a lentiviral vector carrying an anti-PD-1 antibody (pembrolizumab) gene under the control of an NFAT promoter: (3) T/GSI026·anti-PD-1: engineered human primary T cells transduced with a lentiviral vector carrying both an anti-EGFRvIII-CAR gene (GSI026) and an anti-PD-1 antibody (pembrolizumab) gene under the control of an NFAT promoter; and (4) UnT: engineered human primary T cells transduced with an irrelevant gene as a negative control. Additionally, a mixture of engineered human primary T cells transduced with a lentiviral vector carrying an anti-EGFRvIII-CAR gene and engineered human primary T cells transduced with a lentiviral vector carrying an anti-PD-1 antibody gene under the control of an NFAT promoter can be prepared and assessed for cytotoxicity against tumor cells.

Each group of engineered primary human T cells (“effector cells”) was co-cultured with U87MG/VIII-Luc-PD-L1 cells (“target cells”) over-expressing PD-L1 at different E/T ratios (e.g. 5:1, 10:1, 20:1) for 5 days. Cytotoxicity of the antibody-secreting primary human T cells on tumor cells was monitored by determining the remaining luciferase activity using the ONE-GLO™ luminescent assay kit according to the manufacturer's protocol. A low RLU value in the assay indicates strong cytotoxic efficacy of engineered T cells against U87MG/VIII-Luc-PD-L1 cells. In the same assay format, anti-PD-1 antibody and INF-gamma secretions in the co-culture assay were determined using the corresponding HTRF kits. Additionally, IL-2 secretion can be monitored in the co-culture assay.

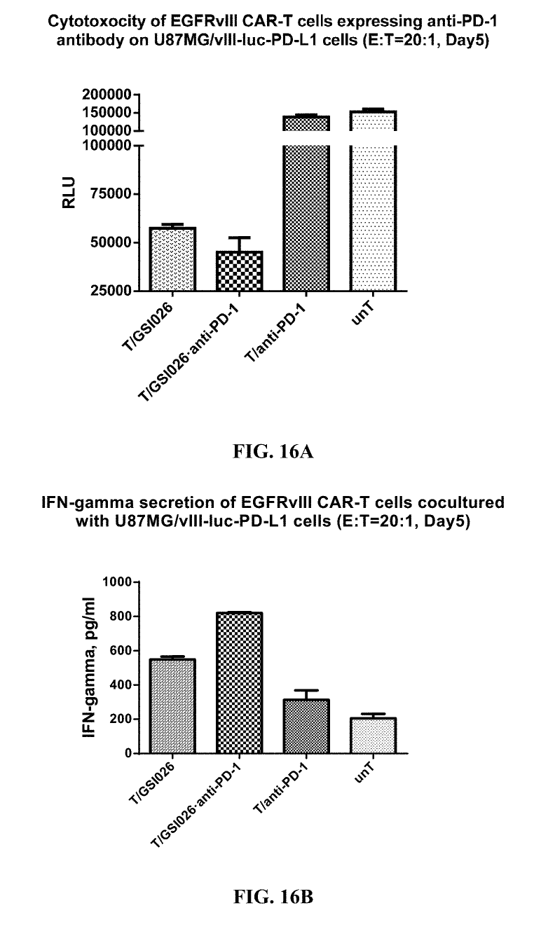

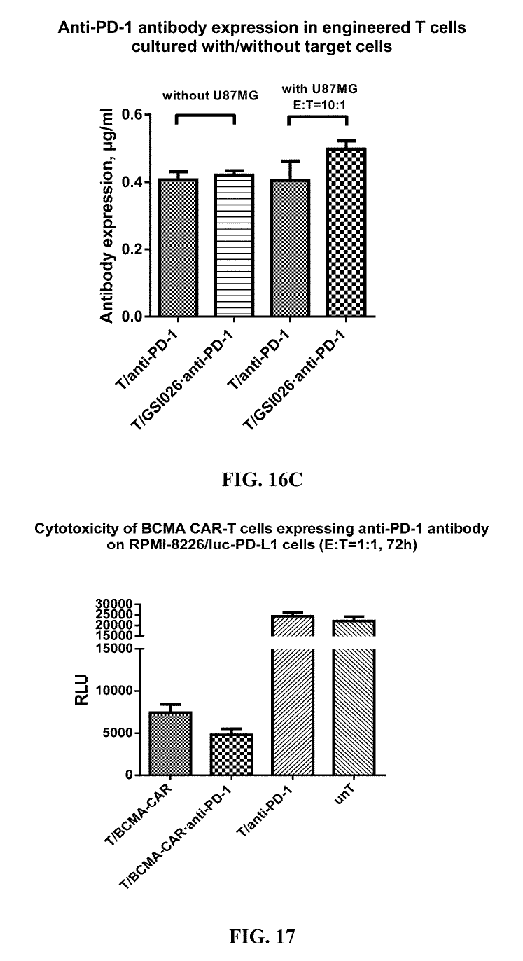

As shown in FIG. 16A, when co-cultured with target cells for 5 days, CAR-T cells expressing both anti-EGFRvIII-CAR and anti-PD-1 antibody (T/GSI026-anti-PD-1) showed more potent cytotoxicity (RLU=45151±7385) against U87MG/VIII-Luc-PD-L1 tumor cells than CAR-T cells expressing anti-EGFRvIII-CAR alone (T/GSI026, RLU=57474±1922) or T cells expressing anti-PD-1 antibody (T/anti-PD-1, RLU=138549±5625) alone. As shown in FIG. 16B, after 5 days of co-culture, CAR-T cells expressing both anti-EGFRvIII-CAR and anti-PD-1 antibody (T/GSI026·anti-PD-1) secreted higher levels of IFN-gamma (820.66±3.24 μg/mL) than CAR-T cells expressing anti-EGFRvIII-CAR alone (548.98±12.39 μg/mL) or T cells expressing anti-PD-1 antibody (314.06±38.64 μg/mL) alone.

Anti-PD-1 antibody secretion is shown in FIG. 16C. When co-cultured with U87MG/vIII-luc-PD-L1 cells at an E/T ratio of 10:1 for 3 day s, CAR-T cells expressing both anti-EGFRvIII-CAR and anti-PD-1 secreted more anti-PD-1 antibody (0.50±0.02 μg/mL) than T cells expressing anti-PD-1 alone (0.40±0.04 μg/mL) or CAR-T cells that were not co-cultured with target cells (0.42±0.01 μg/mL). This result suggests that co-culturing with the target cell induces the NFAT promoter in the CAR-T cells, which enhances the expression of the anti-PD-1 antibody gene driven by the NFAT promoter, and in turn enhances cytotoxicity of the engineered T cells against the target cells.

Such enhanced cytotoxicity of CAR-T expressing an anti-PD-1 antibody gene driven by an NFAT promoter was also observed in another in vitro CAR-T killing model, in which the target of the CAR is a different tumor antigen, BCMA. As shown in FIG. 17, human primary T cells transduced with lentiviral vectors carrying both an anti-BCMA-CAR gene and an anti-PD-1 antibody gene (T/BCMA-CAR·anti-PD-1) potently inhibited the growth of RPM1-8226/Luc-PD-L1 tumor cells, which highly expressed tumor-associated antigen BCMA and inhibitory checkpoint molecule ligand PD-L1. The CART cells expressing both anti-BCMA-CAR and the anti-PD-1 antibody had higher cytotoxicity (RLU: 4807±698) than CAR-T cells expressing anti-BCMA-CAR alone (RLU: 7429±971) or T cells expressing anti-PD-1 antibody alone (RLU: 24398±1875).

These data indicate that anti-PD-1 antibody expressed by engineered primary human T cells enhances CAR-T cytotoxicity against tumor cells in vitro.

Similarly, to assess the effects of other promotes for the anti-PD-1 antibody on anti-tumor efficacy of the engineered T cells, other promoters such as a hEF1α promoter, a doxycycline inducible promoter (e.g., TETON®), or a heat inducible promoter (e.g., HSP70p) can be used in place of the NFAT promoter in the above experiments. CAR-T cells expressing both anti-EGFRvIII-CAR and anti-PD-1 under such promoters may also show higher potency in killing U87M G/VIII-Luc-PD-L1 tumor cells than CA R-T cells expressing anti-EGFRvIII-CAR alone or primary T cells expressing anti-PD-1 antibodies alone. Such effect may be dose dependent on the inducer, such as doxycycline, if the transduced anti-PD-1 gene is under the control of Tet-On system. Such effect may depend on the heat-shock temperature or duration, if the transduced anti-PD-1 gene is under the control of HSP70p.

### Example 4: Anti-CTLA-4 Antibody Expressed by Engineered Primary Human T Cells Augments CART Cytotoxicity Against Human Tumor Cells In Vitro

The following experimental groups of T cells were prepared: (1) T/GSI026: engineered human primary T cells transduced with a lentiviral vector carrying an anti-EGFRvIII-CAR gene (GSI026); (2) T/anti-CTLA-4: engineered human primary T cells transduced with a lentiviral vector carrying an anti-CTLA-4 antibody (Ipilimumab) gene under the control of an NFAT promoter, (3) T/GSI026·anti-CTLA-4: engineered human primary T cells transduced with a lentiviral vector carrying both an anti-EGFRvIII-CAR gene (GSI026) and an anti-CTLA-4 antibody (Ipilimumab) gene under the control of an NFAT promoter; and (4) unT: engineered human primary T cells transduced with an irrelevant gene as a negative control. Additionally, a mixture of engineered human primary T cells transduced with a lentiviral vector carrying an anti-EGFRvIII-CAR gene and engineered human primary T cells transduced with a lentiviral vector carrying an anti-CTLA-4 antibody gene under the control of an NFAT promoter can be prepared and assessed for cytotoxicity against tumor cells.

Each group of engineered primary human T cells (“effector cells”) was co-cultured with U87MG/VIII-Luc-CD80/CD86 cells (“target cells”) over-expressing CD80/CD86 at different E/T ratios (e.g. 5:1, 10:1, 20:1) for 5 days. Cytotoxicity of the antibody-secreting primary human T cells on tumor cells was monitored by determining the remaining luciferase activity using the ONE-GLO™ luminescent assay kit according to the manufacturer's protocol. A low RLU value in the assay indicates strong cytotoxic efficacy of engineered T cells against U87M GA/Ill-Luc-CD80/CD86 cells. In the same assay format, anti-CTLA-4 antibody, INF-gamma, and IL-2 secretions in the co-culture assay can be determined using the corresponding HTRF kits.

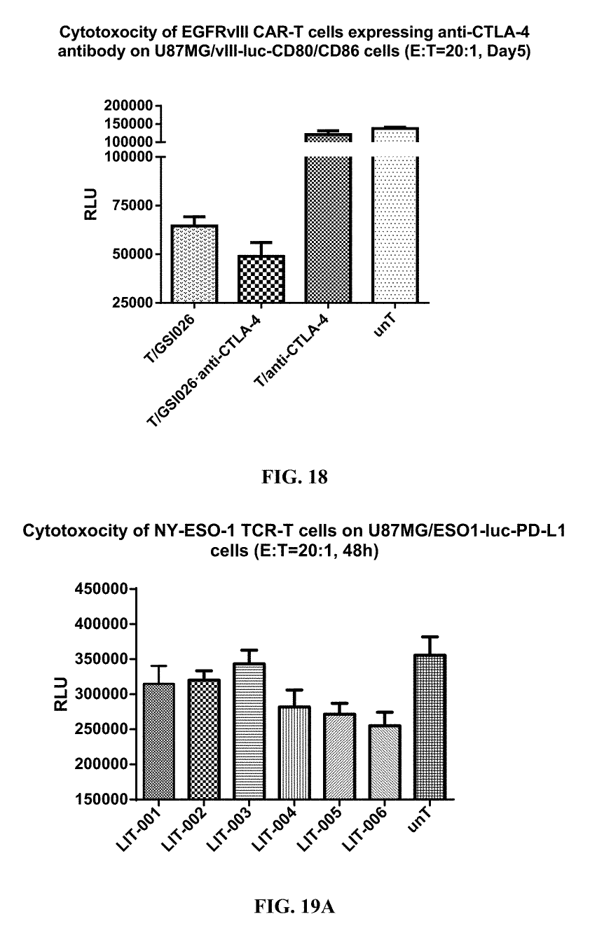

As shown in FIG. 18, when co-cultured with target cells for 5 days, CAR-T cells expressing both anti-EGFRvIII-CAR and anti-CTLA-4 antibody (T/GSI026·anti-CTLA-4) showed more potent cytotoxicity (RLU=48980±7063) against U87MG/VIII-Luc-CD80/CD86 tumor cells than CAR-T cells expressing anti-EGFRvIII-CAR alone (T/GSI026, RLU-64575±4706) or T cells expressing anti-CTLA-4 antibody alone (T/anti-CTLA-4, RLU: 120836±10424). These data indicate that anti-CTLA-4 antibody expressed by engineered primary human T cells enhances CAR-T cytotoxicity against tumor cells in vitro.

Similarly, to assess the effects of other promoters for the anti-CTLA-4 antibody on anti-tumor efficacy of the engineered T cells, other promoters such as a hEF1α promoter, a doxycycline inducible promoter (e.g., TETON®), or a heat inducible promoter (e.g., HSP70p) can be used in place of the NFAT promoter in the above experiments. Primary T cells expressing both anti-EGFRvIII-CAR and anti-CTLA-4 under such promoters may also show higher potency in killing U87MG/VIII-Luc-CD80/CD86 tumor cells than CAR-T cells only expressing anti-EGFRvIII-CAR alone or T cells expressing anti-CTLA-4 antibodies alone. Such effect may be dose dependent on the inducer, such as doxycycline, if the transduced anti-CTLA-4 gene is under the control of Tet-On system. Such effect may depend on the heat-shock temperature or duration, if the transduced anti-CTLA-4 gene is under the control of HSP70p.

### Example 5: Anti-PD-1 Antibody Expressed by Engineered Primary Human T Cells Augments CART Cytotoxicity Against Human Solid Tumor In Vivo

The in vivo efficacies of engineered human primary T cells expressing anti-PD-1 antibody alone or in combination with chimeric antigen receptors (CAR) can be evaluated in a mouse xenograft model, in which human tumor cells are implanted. For example, U87MG/VIII-Luc-PD-L1 tumor cells are implanted into a group of NSG mice to provide a mouse xenograft model of human glioblastoma.

Engineered human primary T cells are prepared with different transduction protocols as described in Example 1. The modelized mice are infused with each of the following groups of cells for treatment: (1) engineered human primary T cells transduced with a lentiviral vector carrying an anti-EGFRvIII-CAR gene; (2) engineered human primary T cells transduced with a lentiviral vector carrying an anti-PD-1 antibody gene; (3) a mixture of engineered human primary T cells transduced with a lentiviral vector carrying an anti-EGFRvIII-CAR gene and engineered human primary T cells transduced with a lentiviral vector carry Mg an anti-PD-1 antibody gene; (4) engineered human primary T cells transduced with a lentiviral vector carrying both an anti-EGFRvIII-CAR gene and an anti-PD-1 antibody gene; and (5) engineered human primary T cells transduced with an irrelevant gene as a negative control. The anti-PD-1 antibody gene is under the transcriptional control of a doxycycline inducible promoter (e.g., TETON®), or an NFAT promoter. Secretion of anti-PD-1 antibody in each treatment condition is induced either prior to administration to the mice, or after administration to the mice under suitable conditions.

Efficacy of each treatment condition is assessed by several parameters including remission of tumor cells. Tumor size may be monitored by in vivo bioluminescence imaging before and after the treatment.

Primary T cells expressing both anti-EGFRvIII-CAR and anti-PD-1 antibody may be more potent in killing U87MG/VIII-Luc-PD-L1 tumor cells than primary T cells only expressing anti-EGFRvIII-CAR or primary T cells only expressing anti-PD-1 antibody. Such effect may be dose dependent on doxycycline if the transduced anti-PD-1 gene is under the control of Tet-on system. Such effect may depend on the existence of EGFRvIII antigen-specific CAR-T, if the expression of transduced anti-PD-1 gene is under the control of an NFAT-dependent inducible promoter. Such effect may depend on the heat-shock temperature or duration, if the transduced anti-PD-1 gene is under the control of HSP70p.

### Example 6: Anti-CTLA-4 Antibody Expressed by Engineered Primary Human T Cells Augments CART Cytotoxicity Against Human Solid Tumor In Vivo

The in vivo efficacies of engineered human primary T cells expressing anti-CTLA-4 antibody alone or in combination with chimeric antigen receptors (CAR) can be evaluated in a mouse xenograft model, in which human tumor cells are implanted. For example, U87MG/VIII-Luc-CD80/CD86 tumor cells are implanted into a group of NSG mice to provide a mouse xenograft model of human glioblastoma.

Engineered human primary T cells are prepared with different transduction protocols as described in Example 1. The modelized mice are infused with each of the following groups of cells for treatment: (1) engineered human primary T cells transduced with a lentiviral vector carrying an anti-EGFRvIII-CAR gene; (2) engineered human primary T cells transduced with a lentiviral vector carrying an anti-CTLA-4 antibody gene; (3) a mixture of engineered human primary T cells transduced with a lentiviral vector carrying an anti-EGFRvIII-CAR gene and engineered human primary T cells transduced with a lentiviral vector carrying an anti-CTLA-4 antibody gene; (4) engineered human primary T cells transduced with a lentiviral vector carrying both an anti-EGFRvIII-CAR gene and an anti-CTLA-4 antibody gene; and (5) engineered human primary T cells transduced with an irrelevant gene as a negative control. The anti-CTLA-4 antibody gene is under the transcriptional control of a doxycycline inducible promoter (e.g., TETON®), or an NFAT promoter. Secretion of anti-CTLA-4 antibody in each treatment condition is induced either prior to administration to the mice, or after administration to the mice, under suitable conditions.

Efficacy of each treatment condition is assessed by several parameters including remission of tumor cells. Tumor size may be monitored by in vivo bioluminescence imaging before and after the treatment.

Primary T cells expressing both anti-EGFRvIII-CAR and anti-CTLA-4 antibody may be more potent in killing U87MG/VIII-Luc-CD80/CD86 tumor cells than primary T cells only expressing anti-EGFRvIII-CAR or primary T cells only expressing anti-CTLA-4 antibody. Such effect may be dose dependent on doxycycline if the transduced anti-CTLA-4 gene is under the control of Tet-on system. Such effect may depend on the existence of EGFRvIII antigen-specific CAR-T, if the expression of transduced anti-CTLA-4 gene is under the control of an NFAT-dependent inducible promoter. Such effect may depend on the heat-shock temperature or duration, if the transduced anti-CTLA-4 gene is under the control of HSP70p.

### Example 7: Anti-PD-1 Antibody Expressed by Engineered Primary Human T Cells Augments CART Cytotoxicity Against Human Liquid Tumor In Vivo

The in vivo efficacies of engineered human primary T cells expressing anti-PD-1 antibody alone or in combination with a CAR can be evaluated in a mouse xenograft model, in which human tumor cells are implanted. For example, human multiple myeloma cell RPMI-8226 cells engineered to express luciferase transgene are implanted into a group of NSG mice to provide a mouse xenograft model of human multiple myeloma.

Engineered human primary T cells are prepared with different transduction protocols as described in Example 1. The modelized mice are infused with each of the following groups of cells for treatment: (1) engineered human primary T cells transduced with a lentiviral vector carrying an anti-BCMA-CAR gene; (2) engineered human primary T cells transduced with a lentiviral vector carrying an anti-PD-1 antibody gene; (3) a mixture of engineered human primary T cells transduced with a lentiviral vector carrying an anti-BCMA-CAR gene and engineered human primary T cells transduced with a lentiviral vector carrying an anti-PD-1 antibody gene; (4) engineered human primary T cells transduced with a lentiviral vector carrying both an anti-BCMA-CAR gene and an anti-PD-1 antibody gene; and (5) engineered human primary T cells transduced with an irrelevant gene as a negative control. The anti-PD-1 antibody gene is under the transcriptional control of a doxycycline inducible promoter (e.g., TETON®), or an NFAT promoter. Secretion of anti-PD-1 antibody in each treatment condition is induced either prior to administration to the mice, or after administration to the mice, under suitable conditions.

Efficacy of each treatment condition is assessed by several parameters including remission of tumor cells. Tumor size may be monitored by in vivo bioluminescence imaging before and after the treatment.

Primary T cells expressing both anti-BCMA-CAR and anti-PD-1 antibody may be more potent in killing RPMI-8226-Luc tumor cells than primary T cells only expressing anti-BCMA-CAR or primary T cells only expressing anti-PD-1 antibody. Such effect may be dose dependent on doxycycline if the transduced anti-PD-1 gene is under the control of Tet-on system. Such effect may depend on the existence of BCMA antigen-specific CAR-T, if the expression of transduced anti-PD-1 gene is under the control of an NFAT-dependent inducible promoter. Such effect may depend on the heat-shock temperature or duration, if the transduced anti-PD-1 gene is under the control of HSP70p.

### Example 8: Anti-CTLA-4 Antibody Expressed by Engineered Primary Human T Cells Augments CART Cytotoxicity Against Human Liquid Tumor In Vivo

The in vivo efficacies of engineered human primary T cells expressing anti-CTLA-4 antibody alone or in combination with a CAR can be evaluated in a mouse xenograft model, in which human tumor cells are implanted. For example, human multiple myeloma cell RPMI-8226 cells engineered to express luciferase transgene are implanted into a group of NSG mice to provide a mouse xenograft model of human multiple myeloma.

Engineered human primary T cells are prepared with different transduction protocols as described in Example 1. The modelized mice are infused with each of the following groups of cells for treatment: (1) engineered human primary T cells transduced with a lentiviral vector carrying an anti-BCMA-CAR gene; (2) engineered human primary T cells transduced with a lentiviral vector carrying an anti-CTLA-4 antibody gene; (3) a mixture of engineered human primary T cells transduced with a lentiviral vector carrying an anti-BCMA-CAR gene and engineered human primary T cells transduced with a lentiviral vector carrying an anti-CTLA-4 antibody gene; (4) engineered human primary T cells transduced with a lentiviral vector carry in g both an anti-BCMA-CAR gene and an anti-CTLA-4 antibody gene; and (5) engineered human primary T cells transduced with an irrelevant gene as a negative control. The anti-CTLA-4 antibody gene is under the transcriptional control of a doxycycline inducible promoter (e.g., TETON®) or an NFAT promoter. Secretion of anti-CTLA-4 antibody in each treatment condition is induced either prior to administration to the mice, or after administration to the mice, under suitable conditions.

Efficacy of each treatment condition is assessed by several parameters including remission of tumor cells. Tumor size may be monitored by in vivo bioluminescence imaging before and after the treatment.

Primary T cells expressing both anti-BCMA-CAR and anti-CTLA-4 antibody may be more potent in killing RPM1-8226-Luc tumor cells than primary T cells only expressing anti-BCMA-CAR or primary T cells only expressing anti-CTLA-4 antibody. Such Effect may be dose dependent on doxycycline if the transduced anti-CTLA-4 gene is under the control of Tet-on system. Such effect may depend on the existence of BCMA antigen-specific CAR-T, if the expression of transduced anti-CTLA-4 gene is under the control of an NFAT-dependent inducible promoter. Such effect may depend on the heat-shock temperature or duration, if the transduced anti-CTLA-4 gene is under the control of HSP70p.

### Example 9: Anti-PD-1 Antibody Expressed by Engineered Primary Human T Cells Augments TCR-T Cytotoxicity Against Tumor Cells In Vitro

NY-ESO-1 is highly expressed in multiple myeloma with poor prognosis (see, for example, Blood 105:3939-3944 (2005)). Cell therapies using TCR against NY-ESO-1 have been described, for example, in WO/2005/113595. Adoptive transfer of autologous PBMC transduced with a high affinity TCR directed against an HLA-A*0201-restricted NY-ESO-1 has been tested in clinical setting among patients with metastatic synovial cell sarcoma and metastatic melanoma (see, for example, J. Clin. Oncol. 29:917-24 (2011), and Clinical Cancer Research 21:5 (2014)). Rapoport A P et al. (Nat. Med. 21 (8): 914-21 (2015)) also reported encouraging clinical results in a phase I/II clinical trial on multiple myeloma utilizing autologous T cells engineered to express an affinity-enhanced T cell receptor (TCR) recognizing a naturally processed peptide shared by the cancer-testis antigens NY-ESO-1 and LAGE-1. Other undergoing TCR-T immunotherapy clinical trials include, for example, TCR immunotherapy targeting MAGE-A3 for patients with metastatic cancer who are HLA-DP0401 positive (NCT02111850).

Six lentiviral vectors encoding TCRs (LIT-001 to LIT-006, shown in Table 2 below) that recognize the peptide SLLMWITQC, corresponding to residues 157-165 of NY-ESO-1(NY-ESO-1:157-165), in the context of the HLA-A*0201 class 1 restriction element, were design al. V alpha and V beta sequences (wildtype variant 1G4 and affinity matured variant 113-1G4) were designed according to Li Y, Nature biotechnology. 2005; 23:349-354 and Robbins P. F. et al, J Immunol. 2008 May 1; 180(9): 6116-6131. Sequences of the constant region of both chains were designed according to sequences from UniProt: TCA: accession number P01848; TRBC1: accession number: P01850; and TRBC2: accession number A0A5B9. TCR alpha chain and TCR beta chain sequences were linked by a T2A peptide (PLoS ONE 6(4): e18556). Both chains were leaded by a 1G4 native signal peptide or a CD8a signal peptide sequence. The designed TCR sequences were further codon optimized to allow optimal expression on human cells. The nucleotide sequences were synthesized and cloned into a lentiviral vector, which was modified from PLVX-Puro (Clontech#632164) by replacing the original promoter with human elongation factor 1α promoter (hEF1α) and deleting puromycin resistance gene. The TCR-encoding vectors (pLLV-LIT001 to pLLV-LIT006) were produced with lentiviral packaging systems in 293T cells as described in above examples.

TABLE 2
Constructed vectors

| | Signal | | alpha | | Signal | | |
|---|---|---|---|---|---|---|---|
| TCR | peptide | V alpha | Constant | 2A | peptide | V beta | beta Constant |
| LIT-001 | CD8α | 113-1G4 | TCA | P2A | CD8α SP | 113- | TRBC1 |
| | SP-6His | | (P01848) | | | 1G4 | (P01850) |
| LIT-002 | 1G4 | 113-1G4 | TCA | P2A | 1G4 | 113- | TRBC1 |
| | native | | (P01848) | | native SP | 1G4 | (P01850) |
| | SP-6His | | | | | | |
| LIT-003 | 1G4 | Wt 1G4 | TCA | P2A | 1G4 | Wt 1G4 | TRBC1 |
| | native | | (P01848) | | native SP | | (P01850) |
| | SP-6His | | | | | | |
| LIT-004 | CD8α | 113-1G4 | TCA | P2A | CD8α SP | 113- | TRBC2 |
| | SP-6His | | (P01848) | | | 1G4 | (A0A5B9) |
| LIT-005 | 1G4 | 113-1G4 | TCA | P2A | 1G4 | 113- | TRBC2 |
| | native | | (P01848) | | native SP | 1G4 | (A0A5B9) |
| | SP-6His | | | | | | |
| LIT-006 | 1G4 | Wt 1G4 | TCA | P2A | 1G4 | Wt 1G4 | TRBC2 |
| | native | | (P01848) | | native SP | | (A0A5B9) |
| | SP-6His | | | | | | |

Primary human T cells were isolated by magnetic bead isolation from PBMC from apheresis blood samples of donor, following manufacturer's instructions (Human PanT isolation kit, Miltenyi #130-096-535). Primary T cells were pre-activated with human T Cell Activation/Expansion Kit (Miltenyi #130-091-441) for 2 days. The pre-activated T cells were then transduced with the above TCR-encoding lentiviral vectors to prepare TCR-T cells expressing TCRs LIT-001 to LIT-006.

Human malignant glioma cell line U87MG cells expressing NY-ESO-1, HLA-A*0201 and PD-L1 as well as the reporter luciferase were prepared, and referred herein as U87MG/ESO1-luc-PD-L1 tumor cells. Additional tumor cell lines expressing NY-ESO-1 have been described. See, for example, J. Immunother. 37:135-16 (2014). Such cell lines can be used to assess the engineered primary human T cells in similar cytotoxicity assays. For example, human multiple myeloma cell line RPMI8226 cells expressing NY-ESO-1 and HLA-A*0201 as well as the reporter luciferase can be prepared to provide reporter tumor cell line RPMI8226-Luc/NY-ESO-1-A2 for assessment of TCR-T cell cytotoxicity.

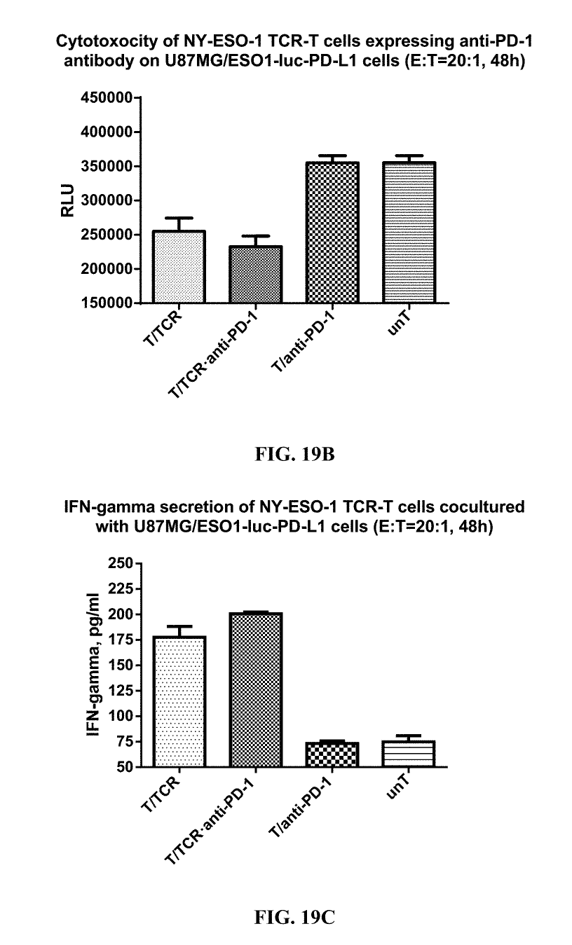

To assess cytotoxicity of the TCR-T cells, the TCR-T cells were each co-cultured with the U87MG/ESO1-luc-PD-L1 tumor cells at an E/T ratio of 20:1 for 48 hours. Cytotoxicity of the TCR-T cells against tumor cells was determined using the ONE-GLO™ luminescent assay kit as described above. As shown in FIG. 19A, TCR-T cells expressing LIT-001˜LIT-006 showed varying potency of cytotoxicity against U87MG/ESO1-luc-PD-L1 tumor cells. Among the six TCR constructs, LIT-006 T cells had the highest potency (RLU=255171±19251) corresponding to 28.25% cytotoxicity. Therefore, TCR-T cells expressing LIT-006 TCR were chosen as an example to assess effects of anti-PD-1 expression on the TCR-T efficacy against tumor cells.

Lentiviral vectors carrying an anti-PD-1 gene under the transcriptional control of a hEF1α promoter (i.e., pLLV-hEF1α-anti-PD-1) were also produced with lentiviral packaging systems as described in Example 1.

In order to determine whether anti-PD-1 antibody expressed by engineered primary human T cells could augment the cytotoxicity of TCR-T, each of the following group of engineered primary human T cells was co-cultured with the U87MG/ESO1-luc-PD-L1 tumor cells at an FIT ratio of 20:1 for 48 h: (1) T/TCR: engineered human primary T cells transduced with the lentiviral vector pLLV-LIT-006 carrying an anti-NY-ESO-1-TCR gene; (2) T/anti-PD-1: engineered human primary T cells transduced with a lentiviral vector carrying an anti-PD-1 antibody gene under the transcriptional control of a hEF1α promoter; (3) T/TCR·anti-PD-1: engineered human primary T cells transduced with a lentiviral vector carrying both an anti-NY-ESO-1-TCR gene (pLLV-LIT-006) and an anti-PD-1 antibody gene under the transcriptional control of a hEF1α promoter; and (4) unT: engineered human primary T cells transduced with an irrelevant gene as a negative control. Additionally, a mixture of engineered human primary T cells transduced with a lentiviral vector carrying an anti-NY-ESO-1-TCR gene and engineered human primary T cells transduced with a lentiviral vector carrying an anti-PD-1 antibody gene can be prepared and assessed for cytotoxicity against the tumor cells.

As shown in FIG. 19B, TCR-T cells expressing bot h anti-NY-ESO-1-TCR and anti-PD-1 (T/TCR⋅anti-PD-1) were more potent (RLU=232567±15464) in killing U87MG/ESO1-luc-PD-L1 tumor cells than TCR-T cells expressing anti-NY-ESO-1-TCR alone (RLU=255171±19251) or T cells expressing anti-PD-1 antibody alone (RLU=355109±10510). Correspondingly, as shown in FIG. 19C, after 48 hours of co-culture with U87MG/ESO1-luc-PD-L1 tumor cells, TCR-T cells expressing both anti-NY-ESO-1-TCR and anti-PD-1 (T/TCR⋅anti-PD-1) secreted a higher level of IFN-gamma (200.77±1.13 μg/mL) than TCR-T cells expressing anti-NY-ESO-1-TCR alone (177.52±7.68 μg/mL) or T cells expressing anti-PD-1 antibody alone (73.19±1.88 pg/mL).

These data indicate that anti-PD-1 antibody expressed by engineered primary human T cells enhances TCR-T cytotoxicity against tumor cells in vitro.

Similarly, to assess the effects of other promoters for the anti-PD-1 antibody on anti-tumor efficacy of the engineered T cells, other promoters such as a doxycycline inducible promoter (e.g., TETON®), a heat inducible promoter (e.g., HSP70p) or an NEAT promoter can be used in place of the hEF1α promoter in the above experiments. TCR-T cells expressing both anti-NY-ESO-1-TCR and anti-PD-1 under such promoters may also show higher potency in killing tumor cells than TCR-T cells expressing anti-NY-ESO-1-TCR alone or primary T cells expressing anti-PD-1 antibodies alone. Such effect may be dose dependent on the inducer, such as doxycycline, if the transduced anti-PD-1 gene is under the control of Tet-On system. Such effect may depend on the heat-shock temperature or duration, if the transduced anti-PD-1 gene is under the control of HSP70p.

### Example 10: Anti-CTLA-4 Antibody Expressed by Engineered Primary Human T Cells Augments TCR-T Cytotoxicity Against Tumor Cells In Vitro

A lentiviral vector encoding a TCR (LIT-006) that recognizes the peptide SLLMWITQC, corresponding to residues 157-165 of NY-ESO-1(NY-ESO-1:157-165), in the context of the HLA-A*0201 class 1 restriction element, was produced with lentiviral packaging systems in 293-6E cells. A lentiviral vector carrying an anti-CTLA-4 gene under the transcriptional control of a hEF1α promoter (i.e., pLLV-hEF1α-anti-CTLA-4) was also produced with lentiviral packaging systems as described in Example 1.

Primary human peripheral blood mononuclear cells (PBMC) were prepared by density gradient centrifugation of peripheral blood from healthy donors. Human primary T cells were purified from PBMCs using magnetic bead isolation.

Human malignant glioma cell line U87MG cells expressing NY-ESO-1, HLA-A*0201 and CD80/CD86 as well as the reporter luciferase were prepared, and referred herein as U87MG/ESO1-luc-CD80/CD86 tumor cells. Other tumor cell lines expressing NY-ESO-1, such as RPMI8226-Luc/NY-ESO-1-A2, can be used for assessment of TCR-T cell cytotoxicity.

Each of the following group of engineered primary human T cells was co-cultured with the U87MG/E901-luc-CD80/CD86 tumor cells for 48 hours: (1) T/TCR: engineered human primary T cells transduced with the lentiviral vector pLLV-LIT-006 carrying an anti-NY-ESO-1-TCR gene; (2) T/anti-CTLA-4: engineered human primary T cells transduced with a lentiviral vector carrying an anti-CTLA-4 antibody gene under the transcriptional control of a hEF1α promoter; (3) T/TCR⋅anti-CTLA-4: engineered human primary T cells transduced with a lentiviral vector carrying both an anti-NY-ESO-1-TCR gene (pLLV-LIT-006) and an anti-CTLA-4 antibody gene under the transcriptional control of a hEF1α promoter; and (4) unT: engineered human primary T cells transduced with an irrelevant gene as a negative control. Additionally, a mixture of engineered human primary T cells transduced with a lentiviral vector carrying an anti-NY-ESO-1-TCR gene and engineered human primary T cells transduced with a lentiviral vector carrying an anti-CTLA-4 antibody gene can be prepared and assessed for cytotoxicity against the tumor cells.

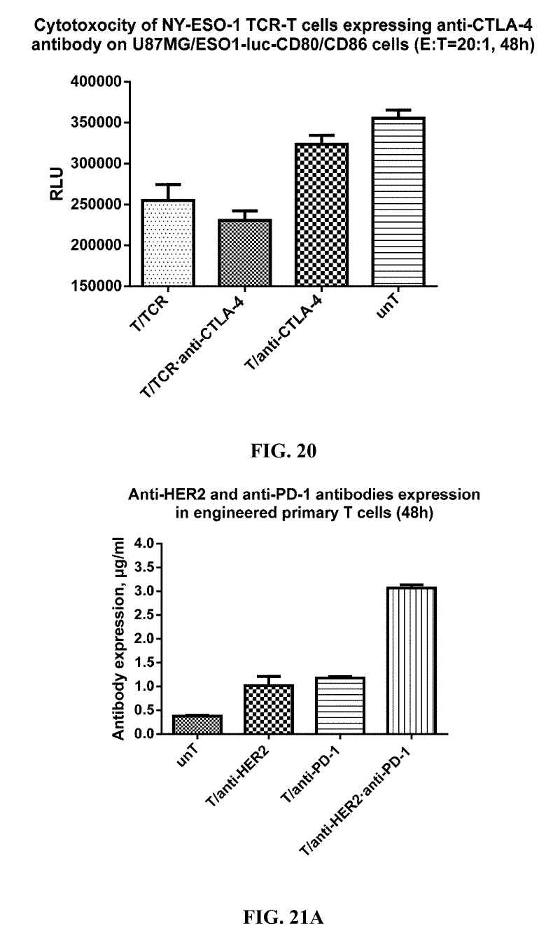

As shown in FIG. 20, TCR-T cells expressing both anti-NY-ESO-1-TCR and anti-CTLA-4 (T/TCR anti-CTLA-4) were more potent (RLU=232567±15464) in killing U87MG/ESO1-luc-CD80/CD86 tumor cells than TCR-T cells expressing anti-NY-ESO-1-TCR alone (RLU=255171±19251) or T cells expressing anti-CTLA-4 antibody alone (RLU=355109±10510). These data indicate that anti-CTLA-4 antibody expressed by engineered primary human T cells enhances TCR-T cytotoxicity against tumor cells in vitro.

Similarly, to assess the effects of other promoters for the anti-CTLA-4 antibody on anti-tumor efficacy of the engineered T cells, other promoters such as a doxycycline inducible promoter (e.g., TETON®), a heat inducible promoter (e.g., HSP70p) or an NFAT promoter can be used in platy of the hEF1α promoter in the above experiments. TCR-T cells expressing both anti-NY-ESO-1-TCR and anti-CTLA-4 antibody under such promoters may also show higher potency in killing tumor cells than TCR-T cells expressing anti-NY-ESO-1-TCR alone or primary T cells expressing anti-CTLA-4 antibody alone. Such effect may be dose dependent on the inducer, such as doxycycline, if the transduced anti-CTLA-4 gene is under the control of Tet-On system Such effect may depend on the heat-shock temperature or duration, if the transduced anti-CTLA-4 gene is under the control of HSP70p.

### Example 11: Co-Expression of Anti-HER2 Antibody and an Immune Checkpoint Inhibitor by Engineered Primary Human T Cells Shows Potent Cytotoxicity Against Tumor Cells In Vitro

Primary human peripheral blood mononuclear cells (PBMC) were prepared by density gradient centrifugation of peripheral blood from healthy donors. Human primary T cells were purified from PBMCs using magnetic bead isolation. The sequences encoding light chain and heavy chain of anti-HER2 antibody were designed and cloned into two pcDNA3.1 plasmid vectors, respectively. 4 μg of each plasmid was transfected into 1×10⁷ human primary T cells by electroporation at 665V and with 43 ms pulse time. The transfected T cells were expanded ex vivo for 3 days. Then part of transfected T cells were transduced with a lentiviral vector carrying an anti-PD-1 antibody (or anti-CTLA-4 antibody) gene to express both anti-HER2 and anti-PD-1 (or anti-CTLA-4) antibodies. Antibody secretion can be detected by HTRF techniques using LANPOWER™ Human Fc Detection kit (GenScript # L00656-1000).

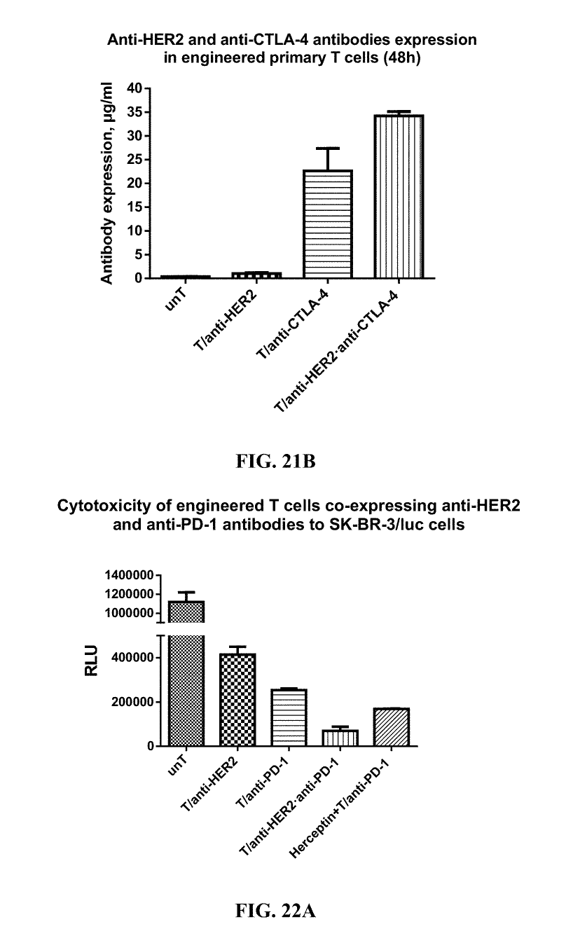

As shown in FIGS. 21A-21B, engineered human primary T cells transfected with plasmids encoding the anti-HER2 antibody gene expressed and secreted 1.02±0.136 μg/mL anti-HER2 antibody when cultured for 48 h. T cells engineered to co-express anti-HER2 and anti-PD-1 (or anti-CTLA-4) antibodies secreted both antibodies with total expression of 3.07±0.046 μg/mL for anti-HER2+ anti-PD-1 (FIG. 21A) and 34.24±0.64 μg/mL for anti-HER2+ anti-CTLA-4 (FIG. 21B).

Bioactivity of the secreted antibodies was assessed in an in vitro tumor cell growth inhibition assay using human breast cancer cell line SK-BR-3, which overexpresses HER2. Here, SK-BR-3 cells are engineered to express a luciferase reporter gene (hereafter referred to as “SK-BR-3/Luc cells”) to allow quantification of live tumor cells by assaying luciferase activity. Anti-HER2 antibody known as Herceptin (Trastuzumab) has potent cytotoxicity against SK-BR-3/Luc cells.

Four groups of engineered human primary T cells were prepared by transducing vectors encoding anti-PD-1 antibody (or anti-CTLA-4 antibody) and anti-HER2 antibody: (1) T/anti-HER2: engineered human primary T cells transfected with plasmids encoding the anti-HER2 antibody gene; (2) T/anti-PD-1 (or T/anti-CTLA-4): engineered human primary T cells transduced with a lentiviral vector carrying an anti-PD-1 antibody (or anti-CTLA-4 antibody) gene; (3) T/anti-HER2·anti-PD-1 (or T/anti-HER2·anti-CTLA-4): engineered human primary T cells transfected with plasmids encoding the anti-HER2 antibody gene and then transduced with lentiviral vectors carrying an anti-PD-1 (or anti-CTLA4) gene; (4) unT: untransduced primary human T cells as negative control. In this experiment, anti-PD-1 and anti-CTLA-4 antibody genes were driven under a hEF1α promoter, while anti-HER2 antibody gene was driven under a hCMVie promoter.

3 days post transduction, each group of engineered T cells was added to a 96-well plate pre-seeded with SK-BR-3/Luc cells at an E:T ratio of 20:1, and co-cultured for 7 days. The remaining luciferase activities in the wells were determined using the ONE-GLO™ luciferase assay kit to assess the cytotoxicity of the engineered T cells against the tumor cells. Low value of relative light unit (RLU) indicates strong cytotoxicity of the engineered T cells against SK-BR-3/Luc cells.

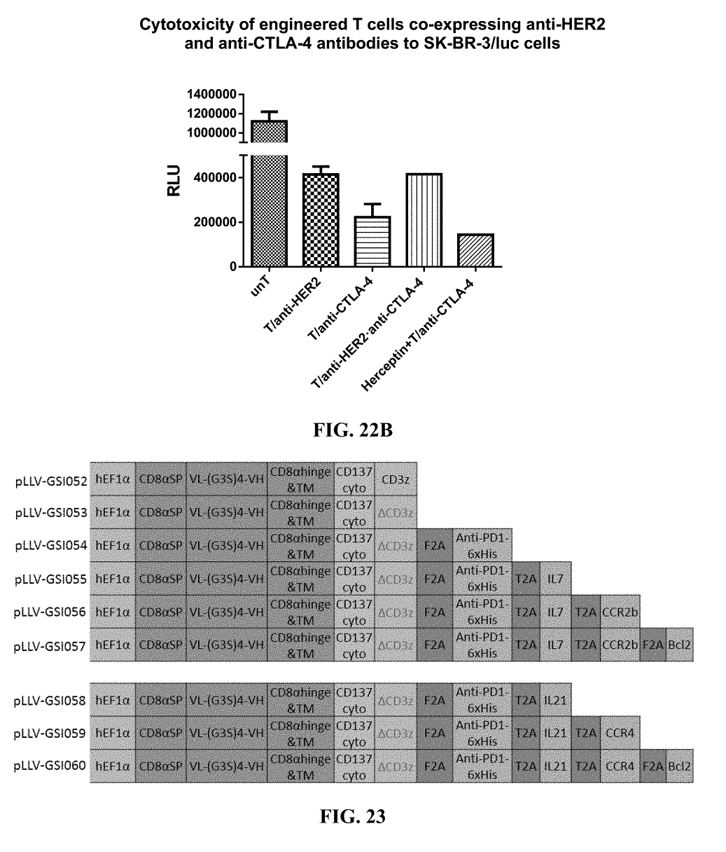

As shown in FIG. 22A, engineered T cells expressing one or both antibodies (anti- and/or anti-PD-1) had higher cytotoxicity against SK-BR-3/Luc cells as than untransduced T cells (UnT). Engineered T cells expressing both anti-HER2 and anti-PD-1 antibodies showed enhanced anti-tumor effects (RLU-69719±13382, 93.78% inhibition compared with UnT) against SK-BR-3/Luc cells compared to T cells expressing anti-HER2 alone (RLU=414317±24894, 63.01% inhibition) or anti-PD-1 alone (RLU=253616±5392, 77.36% inhibition). T cells expressing both anti-HER2 and anti-PD-1 antibodies also had superior anti-tumor effects than the combination of T cells expressing anti-PD-1 alone (T/anti-PD-1) with 20 μg/mL Herceptin. These data indicate that co-expression of anti-HER2 antibody and an immune checkpoint inhibitor (such as anti-PD-1 antibody) by engineered primary T cells has potent cytotoxicity against tumor cells in vitro.

As shown in FIG. 22B, engineered T cells expressing one or both antibodies (anti-HER2 and/or anti-CTLA-4) had higher cytotoxicity against SK-BR-3/Luc cells than untransduced T cells (UnT). However, engineered T cells expressing both anti-HER2 and anti-CTLA-4 antibodies did not show enhanced anti-tumor effects against SK-BR-3/Luc cells as compared to T cells expressing anti-HER2 alone or anti-CTLA-4 alone, or a combination of T cells expressing anti-CTLA-4 with 20 μg/mL Herceptin.

Similarly, to assess the effects of other promoters for the anti-HER2 and anti-PD-1 (or anti-CTLA-4) antibodies on anti-tumor efficacy of the engineered T cells, other promoters such as a doxycycline inducible promoter (e.g., TETON®), a heat inducible promoter (e.g., HSP70p) or an NFAT promoter can be used in place of the hEF1α promoter in the above experiments.

### Example 12: Design of an Anti-EGFR CAR-T Expressing an Immune Checkpoint Inhibitor for Treating Lung Cancer

### Introduction

Lung cancer is the most common cause of cancer mortality globally, responsible for nearly 1 in 5 cancer-related deaths, or an estimated 1.6 million people. Both in the U.S. and China, lung cancer is by far the leading cause of cancer-related death among both men and women. According to the American Cancer Society, it is estimated that more than 221,000 Americans were diagnosed with lung cancer in 2015, and NSCLC accounts for 85% of all lung cancers. Overall, 17.4% of people in the United States diagnosed with lung cancer survive five yews after the diagnosis, while clinical outcomes on average are worse in the developing world.

The majority of lung cancer patients are diagnosed with advanced disease (stage IIIb/IV). For these patients, conventional treatment options including surgery, chemotherapy, and radiation are unlikely to result in cure, although they may significantly improve survival and provide symptom relief. Patients with specific genetic mutations may benefit from targeted therapies such as the epidermal growth factor receptor (EGFR) blockers erlotinib (TARCEVA®), afatinib (GILOTRIF®), and gefitinib (IRESSA®). These drugs block the pro-growth signals from EGFR. These drugs can be used to treat patients with certain mutations in the EGFR gene, which are more common in women and people who have never smoked.

Immunotherapies may offer significant benefit to lung cancer patients, including those for whom other treatments are ineffective. Bevacizumab (AVASTIN®) is a monoclonal antibody that targets vascular endothelial growth factor (VEGF), a protein that helps new blood vessels grow. By preventing tumors from growing new blood vessels, a process called angiogenesis, AVASTIN® leads to nutrient starvation in the tumor cells. Ramucirumab (CYRAMZA®) is another angiogenesis inhibitor that can be used to treat NSCLC. In 2015, two new immunotherapy drugs, nivolumab (OPDIVO®) and pembrolizumab (KEYTRUDA) were approved by the FDA for the treatment of lung cancer. Both nivolumab and pembrolizumab target human PD-1 molecule More recently in 2016, another new immunotherapy drug atezolizumab (TECENTRIQ™) was approved for the treatment of bladder cancer. Atezolizumab targets one of the major PD-1 ligand named PD-L1 molecule. Atezolizumab is now in several Phase III lung cancer clinical trials. Due to its effectiveness and safety in clinical trials, it is believed that atezolizumab would be soon approved for the treatment of lung cancer.

Adoptive cell therapy, or more specifically chimeric antigen receptor modified T cell therapy, has recently been tested in a number of clinical trials for treating lung cancer. Several targets are being explored including NY-ESO-1 (NCT01697527, NCT01967823), VEGFR2 (NCT01218867), MAGE-A3 (NCT02111850), mesothelin (NCT01583686, NCT02414269), and WT1 (NCT02408016). There are also phase 1 studies of T cells genetically engineered to target NY-ESO-1 in combination with the checkpoint inhibitor ipilimumab (NCT02070406). So far, there is little progress of CAR-T targeting such antigens.

EGFR, also known as ErbB-1 or HER1, is one of the receptor in the epidermal growth factor receptor, a subfamily of 4 closely related receptor tyrosine kinases (RTKs): EGFR (ErbB-1), HER2/c-neu (ErbB-2), Her 3 (ErbB-3) and Her 4 (ErbB-4). One of the key natural ligands of EGFR is EGF.

EGFR is over-expressed in about 40%-80% NSCLC (Cancer (2002) 94: 1593-1611; Lancet Oncol. (2003) 4:397-406). Among the various histologic types of lung cancer, EGFR overexpression is most frequent in squamous (84%) and large-cell carcinomas (68%) and least frequent in small-cell lung cancer (The Oncologist(2006) 11:358-373).

Many therapeutic app roaches target EGFR. Cetuximab and panitumumab are examples of monoclonalantibody inhibitors against EGFR. Other anti-EGFR monoclonal antibodies in clinical development are zalutumumab, nimotuzumab, and matuzumab. The monoclonal antibodies block the extracellular ligand binding domain of EGFR

However, EGFR is also widely expressed in a variety of normal tissues at low expression levels. Therefore, cautious design is required for cell therapy strategies targeting EGFR. For example, in other cases, at the early stage of CART development, infusion of ERBB2-specific CART cells constructed using the scFv from the humanized mAb trastuzumab resulted in lethal inflammatory cytokine release in the lung (Morgan R A et al, Mol. Ther. (2010) 18: 843-851). The toxicity was attributed to on-target off-tumor recognition of low levels of ERBB2 expression on lung epithelial cells.

There remains an unmet need for a safe and effective cell therapy against tumors that overexpress EGFR, such as lung cancer.

### Experimental Design

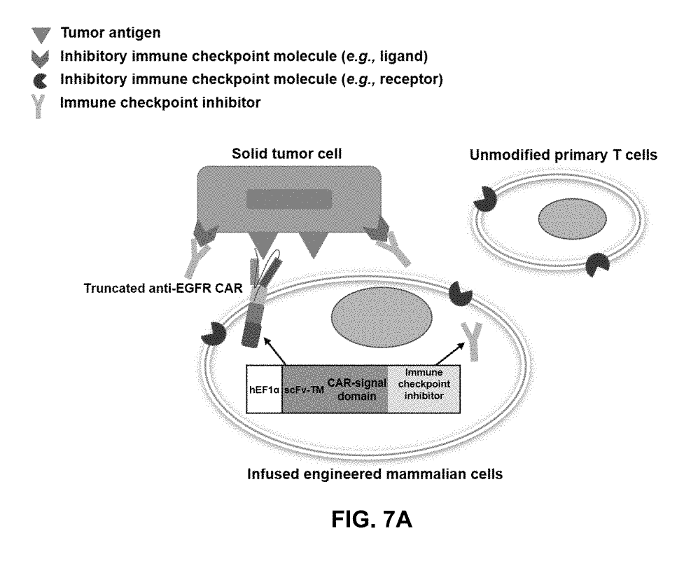

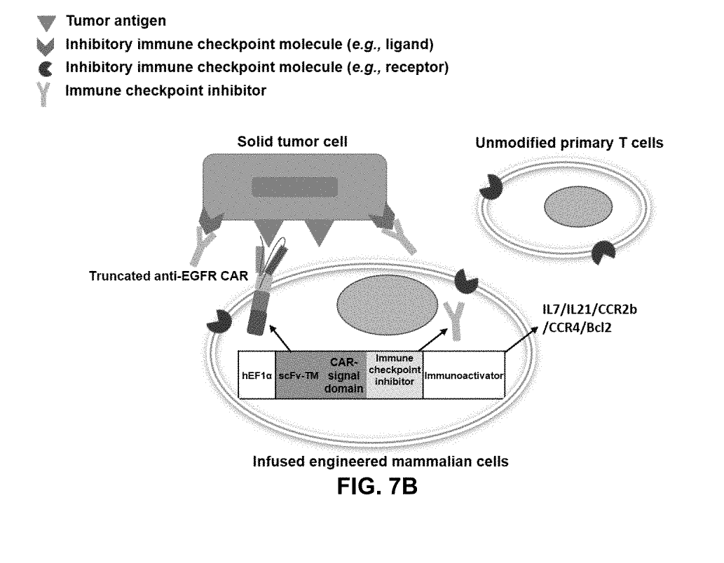

In this example, primary T cells were engineered to co-express a CAR targeting EGFR and an immune checkpoint inhibitor, which are encoded by a single vector (FIG. 7A). The anti-EGFR CAR guides T cells to lung cancer sites that overexpress EGFR, resulting in site-specific expression of the immune checkpoint inhibitor at the lung cancer site. Binding of the anti-EGFR CAR to EGFR on tumor cells activates the truncated or mutated intracellular signaling domain of the CAR, which triggers an attenuated downstream immune response by the engineered T cells, and recruits unmodified immune cells in the host to kill tumor cells, but not normal cells that express low levels of EGFR. The immune response may further be enhanced by engineering the CAR-T to overexpress one or more immunoactivators (FIG. 7B), which, for example, enhance T cell memory, tissue homing, and promotes T cell proliferation and survival.

A panel of vectors encoding the anti-EGFR CARs was designed for preparation of CAR-Ts with improved/vivo safety, persistency, and tissue homing capabilities (FIG. 23). The EGFR binding domain of the anti-EGF R CAR was based on the humanized monoclonal antibody against EGFR clone 425 (mAb425) to guide site-specific expression of immune checkpoint inhibitor antibodies at lung cancer cells expressing EGFR. Construct GS1052 encodes a full-length anti-EGFR CAR, including from the N-terminus to the C-terminus, CD8α signal peptide, mAb425 scFv, CD8α hinge and transmembrane (TM) region, CD137 cytoplasmic domain (CD137 cyto), and CD3.

mAb425 was developed by immunization of BALB/c mice with human A431 cells, a cell line known to highly overexpress EGFR. mAb425 was further humanized to provide matuzumab (see, U.S. Pat. No. 5,558,864). mAb425 binds to EGFR with high affinity. Pre-clinical studies have demonstrated that mAb425 inhibits growth of EGFR dependent tumors, inhibits VEGF expression, and induces ADCC. Matuzumab has undergone phase 11 clinical trials for the treatment of colorectal, lung esophageal and stomach cancer in the early 2000s. However, no further clinical trials have been conducted since the phase 1 trial in 2007. On Feb. 18, 2008, Takeda and Merck announced that they would no longer pursue the development of matuzumab.

As EGFR is widely expressed in normal tissues, to improve in vivo safety of the CAR-T cells, a truncated form of anti-EGFR CAR having a deleted or mutated CD3ζ domain (i.e., CD3z) was designed. For example, construct GS1053 was designed based on GS1052, but GS1053 does not have a CD3ζ domain. GSI053 includes, from the N-terminus to the C-terminus, CD8α signal peptide, mAb425 scFv, CD8α hinge and TM, and CD137 cytoplasmic domain.

Next, a sequence encoding an immune checkpoint inhibitor antibody was cloned next to the sequence encoding the truncated anti-EGFR CAR. For example, in construct GSI054, an anti-PD-1 coding sequence was cloned next to the CD137 cytoplasmic domain. Thus, GSI054 includes, from the N-terminus to the C-terminus, CD8α signal peptide, mAb425 scFv, CD8α hinge and TM, CD137 cytoplasmic domain, and anti-PD-1.

The truncated anti-EGFR CAR can direct expression of immune checkpoint inhibitor antibody to the EGFR-expressing tissues, especially the EGFR overexpressing tumor site, while avoiding excessive killing of target cells. Although the truncated anti-EGFR CAR alone is unable to elicit significant cytotoxicity on EGFR-expressing cells, the truncated anti-EGFR CAR has the potential of inhibiting proliferation of EGFR-overexpressing tumor cells.

To enhance in vivo persistency of infused modified CAR-T cells, one or more immunoactivators, such as IL-7, IL-21 and Bcl2, were engineered in the vectors. For example, constructs GSI055-GSI060 were designed based on GSI054, but GSI055-GSI057 included an additional sequence encoding IL-7, and GS1058-GSI069 included an additional sequence encoding IL-21. IL-7 can mediate homeostasis of nave and memory CD4⁺, CD8⁺ T cells, and IL-7 can also promote hematological malignancies (such as acute lymphoblastic leukemia, T cell lymphoma). IL-21 can promote the maintenance of T (effs). Constructs GSI057 and GSI060 further included an additional sequence encoding Bcl-2. T cells overexpressing Bcl-2 can be more resistant to activation-induced cell death (AICD).

To enhance tissue horning, a sequence encoding CCR2b was included in constructs GSI056 and GSI057, and a sequence encoding CCR4 was included in the construct GS1059 and GSI060. CCR2b-expressing, activated T cells (ATCs) have improved homing (>10-fold) to CCL2-secreting neuroblastoma compared to CCR2 negative ATCs. T lymphocytes co-expressing CCR4 and a chimeric antigen receptor targeting CD30 have improved homing and antitumor activity in a Hodgkin tumor model.

In the multicistronic constructs, the CAR, anti-PD-1 antibody, and optionally one or more immunoactivators were encoded on the same lentiviral vector and driven by the same constitutive promoter hEF1α. Self-cleavable linkers such as F2A and T2A were displaced between different protein-coding sequences to allow efficient multi-gene co-expression.

Human primary T cells were transduced with lentiviral vectors comprising each of the constructs GSI052-GSI060 to provide the CAR-T cells. In vitro efficacies of mAb425-based full-length CAR-T (GSI052) or truncated CAR-T (GS1053) were studied using EGFR overexpressing lung cancer cell line A549-Luc cells. In addition, for CAR-T cells with constructs (GSI054-GSI060), in vitro expression of anti-PD-1, IL-7/IL-21, CCR2b/CCR4, and Bcl2 were determined. The in vivo anti-tumor efficacies are evaluated using an A549-Luc engrafted NSG mouse model.

Non-human primate EGFRs share over 99% protein sequence identity to human EGFR (e.g., NP_005219.2). Carolina Berger et al studied safety of targeting ROR1 in primates with CAR modified T cells (Cancer Immunol. Res. (2015) 3(2): 206). We also designed an in vivo safety study of CAR-T cells transduced with selected anti-EGFR truncated CAR guided anti-PD-1 expression vectors in a non-human primate model.

### In Vitro Cytotoxicity of Anti-EGFR CAR-T on EGFR Overexpressing Lung Cancer Cell Line A549

A549 (ATCC# CCL-185) is a well-known human lung cancer cell line which overexpresses EGFR. In order to facilitate in vitro and in vivo assays, a firefly luciferase gene was introduced to the parental A549 cells, and the derived cell line was named as A549-Luc.

Firstly, an in vitro cytotoxicity assay was performed in order to assess the specificity and bioactivity of the constructed mAb425-based CARs (full-length and truncated). Lentivirus vectors (pLLV-GSI052, pLLV-GS1053, pLLV-GSI057 and pLLV-GSI060 respectively) were packaged as described in the above examples. Lentivirus stocks were prepared after concentrating the supernatant using ultracentrifugation. Human CD3′ T cells were prepared from PBMC using Pan T cell isolation kit (Miltenyi, Cat#130-096-535). The isolated T cells were pre-activated with T cell activation and expansion kit for 3 days. Pre-activated T cells were then transduced with GSI052 or GSI053, GSI057 or GSI060 lentivirus stock, followed by further cell expansion for 3 days. A549-Luc cells were conventionally cultured in F-12K medium supplemented with 10% FBS and 2 μg/ml puromycin.

On day 3 post transduction, transduced T cells were harvested and co-incubated with A549-Luc cells at an effector (CAR-T) to target cells (A549-Luc) of 5:1 for 7 days. ONE-GLO™ luminescent luciferase assay reagents were added to the co-cultured cells to detect the remaining luciferase activity in the wells. Since luciferase is expressed only in A549-Luc cells, the remaining luciferase activity in the well is directly correlated to the number of viable cells in the well. Thus, a low value of relative light units (RLU) in the assay indicates strong cytotoxicity of the CAR-T cells against A549-Luc.

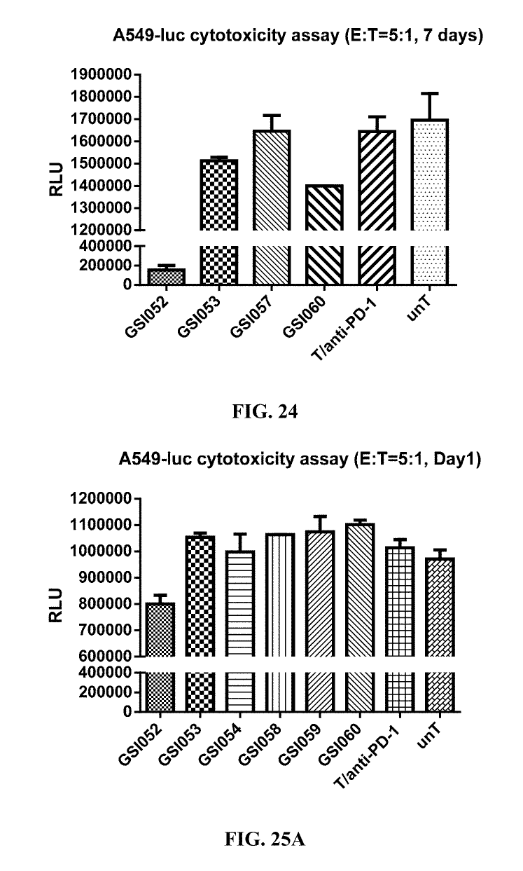

As shown in FIG. 24, CAR-T cells expressing h anti-EGFR-CAR (GSI052) was able to elicit significant cytotoxicity against A549-Luc, while such cytotoxicity was greatly attenuated in CAR-T cells expressing the truncated form of anti-EGFR-CAR (GSI053), in which the CD3ζ domain was absent. Since T cells expressing the truncated CAR (GSI053) could not elicit significant cytotoxicity against EGFR overexpressing cells, normal cells that express low levels of EGFR would not be attacked by the CAR-T cells which might provide improved in vivo safety. CAR-T cells transduced with GSI057 (truncated CAR+ anti-PD-1+IL-7+CCR2b+Bcl2) showed no significant cytotoxicity when co-cultured with A549-luc cells for 7 days, which was unexpected. As IL-7 can promote A549 cell proliferation (Int J Clin Exp Pathol. 2014 Feb. 15; 7(3):870), GS1055-G51057 (expressing IL-7) transduced T cells were not included in the subsequent experiments. T cells transduced with GSI050 (truncated CAR+ anti-PD-1+IL-21+CCR4+Bcl2) showed an enhanced cytotoxicity compared with either GSI053 transduced T cells or T cells only expressing anti-PD-1 antibody (T/anti-PD-1).

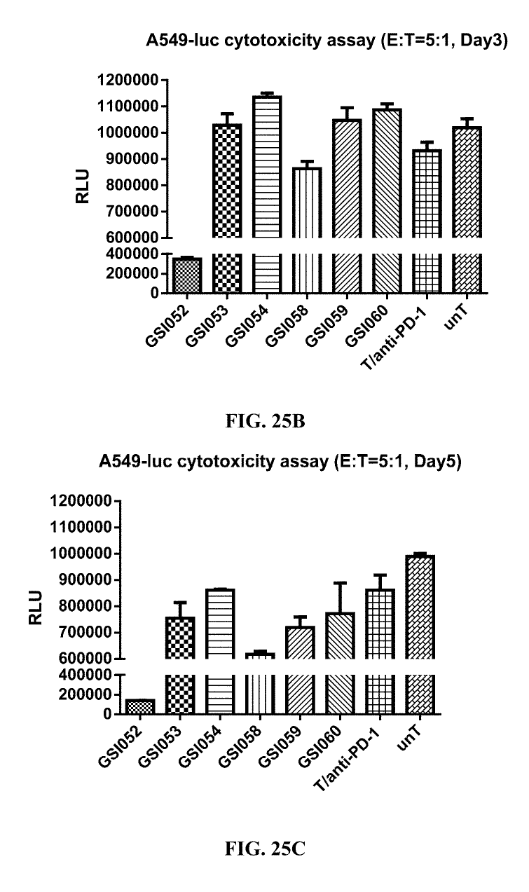

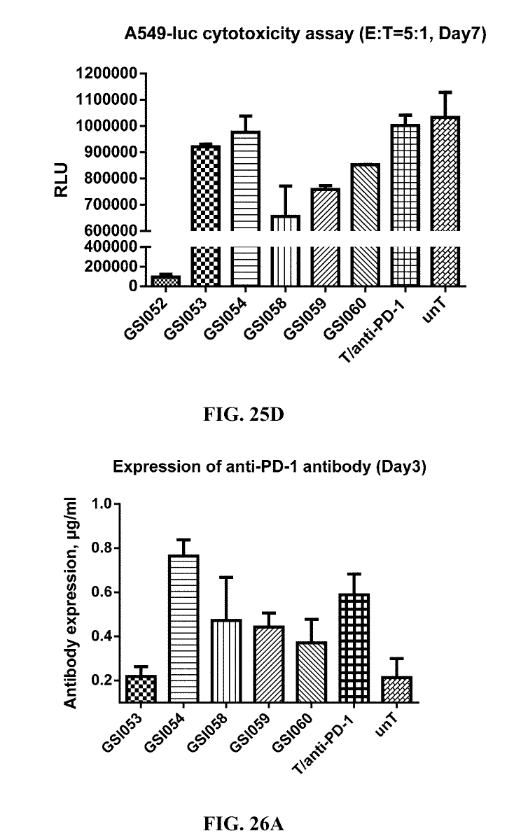

Next, GS1052-G51054 and GS1058-GSI050 transduced CAR-T cells were assessed in similar cytotoxicity assays. On day 3 post transduction, transduced T cells were harvested and co-incubated with A549-Luc cells at an E:T ratio of 5:1. ONE-GLO™ luminescent luciferase assay reagents were added to the co-cultured cells on day 1, 3, 5, or 7 to detect the remaining luciferase activity. As expected, T cells expressing the truncated CAR (GSI053) elicited mild cytotoxicity against A549-Luc lung cancer cells when co-cultured for 5 days or more (FIGS. 25A-25D). GSI058-GSI060 transduced CAR-T cells showed enhanced cytotoxicity compared to untransduced T cells probably due to the expression of anti-PD-1 antibody and IL-21. These results demonstrated that T cells expressing the truncated anti-EGFR CAR alone did not have significant cytotoxic effect against target cells. The introduction of immune checkpoint inhibitor (anti-PD-1 antibody) and immunoactivators such as IL-21 would help to enhance truncated CAR-T functions.

### In Vitro Expression of Anti-PD-1, IL-7/IL-21, CCR2b/CCR4. Bcl2 in Primary Human T Cells

Human primary T cells were prepared, pre-activated, and transduced with each of constructs GSI053, GSI054, GS1058-GSI060. After co-cultured with A549-luc cells for 3 days, the supernatant of transduced primary T cells in each group was collected, and the expression of anti-PD-1 antibody was detected with LANPOWER™ Human Fc Detection kit (GenScript). Briefly, 5 μL of human IgG-GS665, 5 μL of anti-human Fc antibody-Eu, and 10 μL of antibody sample or controls were mixed in an assay plate and incubated at room temperature for 1.5 hours. The plates were read on HTRF compatible instruments (PHERSTAR™ plus microplate reader, Ex 320-340 nm, Em:620 nm and 665 nm).

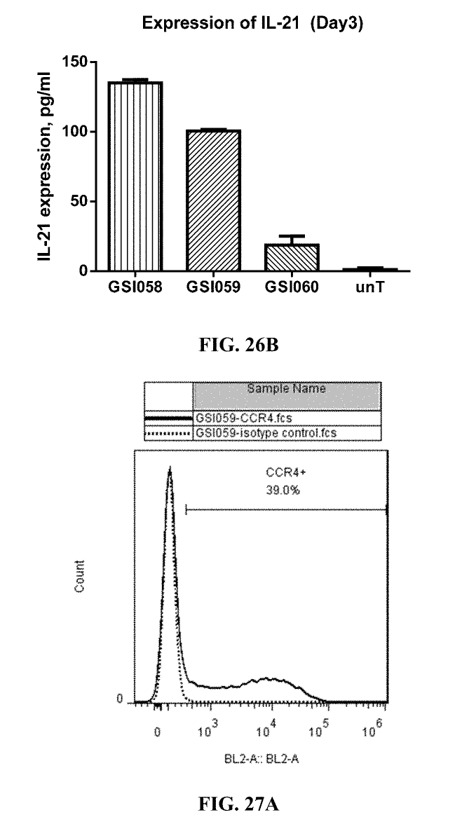

As shown in FIG. 26A, compared with untransduced T cells (UnT) and truncated CAR-T cells (GSI053), T cells transduced with anti-PD-1 antibody gene as well as truncated anti-EGFR-CAR (i.e. GSI054, GSI058, GSI059, GSI060) secreted an increased amount of anti-PD-1 antibody. The expression of anti-PD-1 antibody in GSI054, GS1058, GSI059, GSI060 CAR-T cells were 0.77±0.06 μg/mL, 0.48±0.14 μg/mL, 0.37±0.08 μg/mL, respectively. A lower expression level in GSI060 CAR-T cells could be due to its longer construct (anti-PD-1, IL-21, CCR4, and Bcl2).

IL-21 expression of GS1058-GSI060 engineered primary T cells was detected using Human IL-21 ELISA MAX™ Deluxe kit (Biolegnd#433804) according to the manufacturer's manual. Untransduced T cells (UnT) were used as a negative control. As shown in FIG. 26B, GSI058 CAR-T cells secreted 135.0511.68 pg/mL of IL-21 when co-cultured with A549-luc cells for 3 days, while GSI059 and GSI060 CAR-T cells secreted 100.58±0.80 pg/mL and 18.69±14.58 pg/mL of IL-21, respectively.

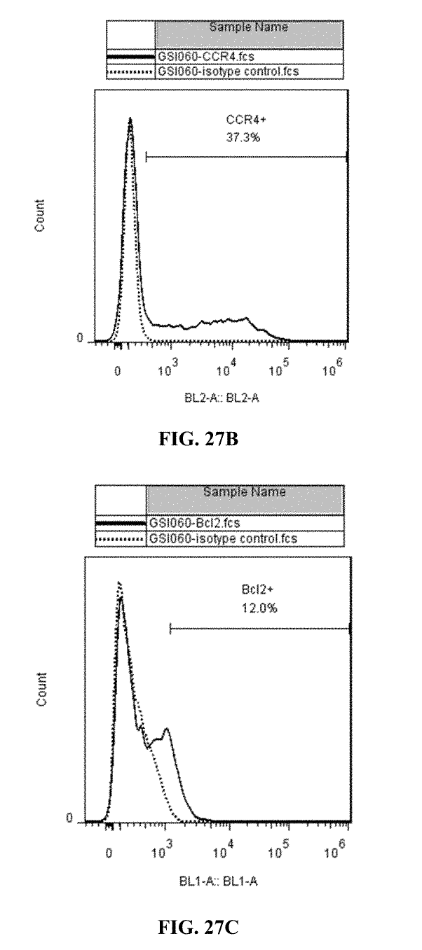

Expression of CCR4 on the cell surface of GSI059 and GSI060 transduced T cells was assessed by flow cytometry using PE labeled anti-human CCR4 (Biolegend#359411) according to instructions by the manufacturer. Briefly, after washing, stained cells were re-suspended in 200 μl DPBS and kept in the dark before applying to flow cytometry analysis on FACSCALIBUR™ (BD Biosciences) or ATTUNENXT™ flow cytometer (Thermo Fisher). CCR4 expression was detected on 39.0% of GSI059 CAR-T cells and 37.3% of GSI060 CAR-T cells (FIGS. 27A-27B).

Expression of Bcl2 protein in G51060 transduced T cells is determined by intracellular staining with ALEXA FLUOR® 488-labeled anti-Bcl-2 (Biolegend#658703) according to instructions by the manufacturer. Briefly, cells were fixed in 0.5 ml/tube of fixation buffer (Biolegend#422601) in the dark for 20 minutes at room temperature. After fixation, the cells were centrifuged at 350 g for 5 min at room temperature and the supernatants were discarded. The cell pellets were re-suspended in intracellular staining permeabilization buffer (Biolegend#422601) and centrifuged at 350 g for 5 min. The cells were rewashed for 5 times. The fixed/permeabilized cells were re-suspended in 200 μL Intracellular Staining Perm Wash Buffer, and ALEXA FLUOR′ 488-labeled anti-Bcl-2 antibodies were added to the cells and incubated for 20 min in the dark at room temperature. After staining cells were washed 2 times with 2 ml of intracellular staining permeabilization buffer and centrifuged at 350 g for 5 mm at room temperature. After washing stained cells were re-suspended in 200 μL DPBS and kept in the dark before applying to flow cytometry analysis on FACSCALIBUR® (BD Biosciences) or ATTUNENXT™ (Thermo Fisher). As shown in FIG. 27C, Bcl2 expression was detected on 12.0% of GSI060 CART cells.

Additional biomarkers on the cell surface of transduced T cells or the tumor cells can be assessed. For example, expression of CCR2b on the cell surface of transduced T cells is assessed by flow cytometry using PE labeled anti-human CCR2 (Biolegend#357205) according to instructions by the manufacturer. Expression of PD-L1 on A549-Luc cells is determined using anti-PD-L1 antibody by flow cytometry.

### In Vivo Anti-Tumor Efficacy of CAR-T Cells Expressing an Anti-PD-1 Antibody in a Human Lung Cancer Model

The in vivo efficacies of engineered human primary T cells expressing anti-PD-1 guided by a truncated anti-EGFR CAR (e.g., GS1053-GSI060) can be evaluated in a mouse xenograft model, in which human tumor cells are implanted. For example, lung cancer cells A549-Luc are implanted in a group of NSG mice to provide a mouse xenograft model of human lung cancer. In the treatment groups, the modelized mice are infused with each group of human primary T cells transduced with lentiviral constructs GS1052-GSI060. In the control group, the modelized mice are infused with untransduced T cells.

Efficacy of each treatment condition is assessed by several parameters including remission of tumor cells. Tumor size may be monitored by in vivo bioluminescence imaging before and after the treatment.

In Vivo Safety of Truncated Anti-EGFR CAR-Guided Anti-PD-1 Treatment in Cynomolgus Monkeys

The in vivo safety of the CAR-T cells expressing truncated anti-EGFR CAR and anti-PD-1 were evaluated in a cynomolgus monkey model.

PBMC was obtained from peripheral blood of two monkeys (NHP#1 and NHP#2, both male, around 4 kg) and prepared by density gradient centrifugation as described above. Cynomolgus monkey T cells were isolated from PBMC using non-human primate Pan T Cell Isolation Kit (Miltenyi #130-091-993) according to the instruction manual. The prepared Monkey T cells were pre-activated with non-human primate T Cell Activation/Expansion Kit (Miltenyi #130-092-919) and human IL-2 with autologous monkey serum for 3 days. Afterwards, the pre-activated T cells were transduced with GSI060 lentivirus, followed by expansion for an extra of 10 days.

The prepared monkey GSI060 transduced T cells were tested for their CAR transgene integration copy number by a real-time PCR with a pair of CAR specific primers. As shown in Table 3, the GSI060 CAR sequence was integrated into the genome of T cells from NHP#2 with 39008.9 copies/ng genomic DNA. The transduction efficiencies can also be analyzed by protein L binding or by detecting transgene expression (such as FACS after staining with anti-CCR2 or anti-CCR4 antibody). Expression of anti-PD-1 can be analyzed by HTRF as described above.

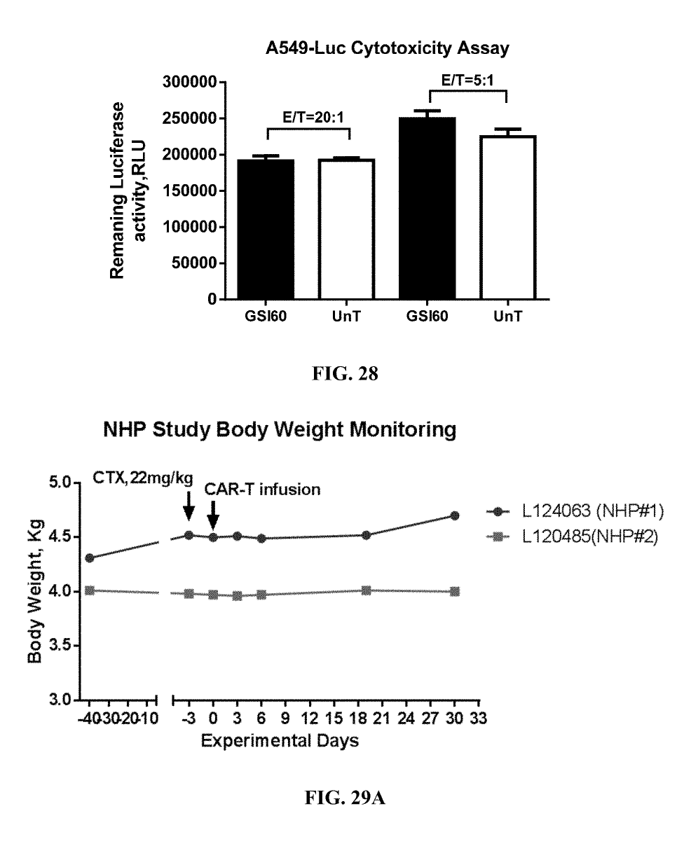

The transduced T cells were also co-cultured with A549-Luc cells overnight. As shown in FIG. 28, there were no significant cytotoxicity of the prepared GSI060 transduced T cells on A549.Luc cells in the co-culture assay.

TABLE 3
Real-time PCR detection of integrated copy numbers of
CAR transgene in NHP CAR-T (Copies/ng genomic DNA)

| | NHP#1 | NHP#2 |
|---|---|---|
| CAR-T | 2151549 | 39008.9 |
| unT | 73.2 | 55.5 |

3 days prior to the infusion of autologous GSI060 modified T cells, the monkeys were pretreated with Cyclophosphamide at a dose of 22 mg/k g body weight by in vein injection. On the day of autologous infusion of GSI060 modified T cells, cells were thawed in a 37° C. water bath by gentle swirling and immediately infused to the animal by in vein infusion within 5 minutes. NHP#2 monkey was infused with 3.2×10⁶/kg GSI060 modified T cells. NHP#1 monkey was infused with a non-related CAR modified T cells.

The monkeys were monitored after the T-cell administrations for fever, respiratory distress, appetite, diarrhea, and weight loss. Pre- and post-administration blood samples were obtained and examined for CBC, serum chemistry, and cytokine levels. For example, plasma levels of IFNγ and IL-6 can be detected on day 1, day 2, day 3 until 4 weeks after the administration.

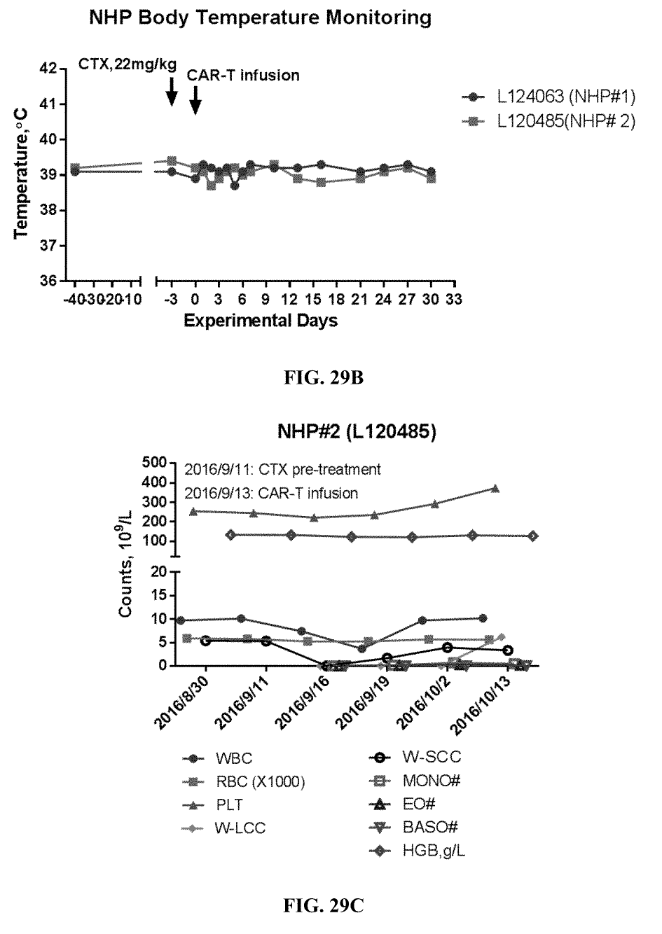

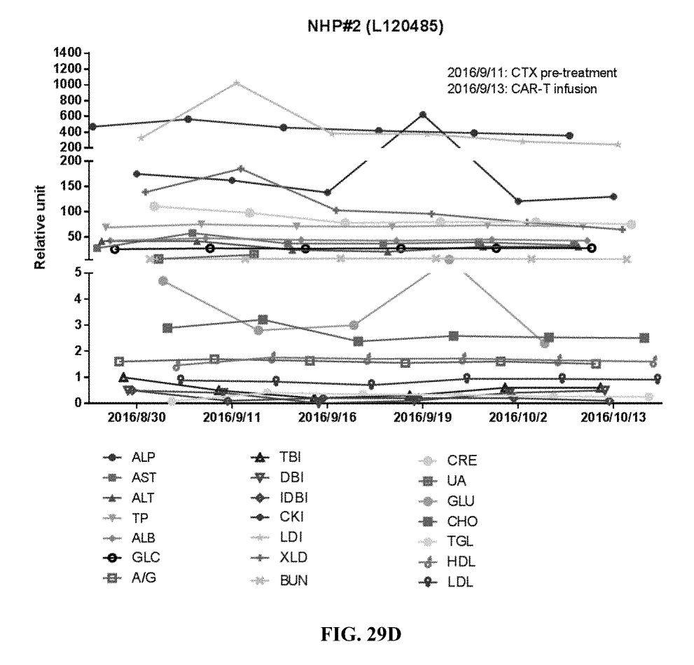

As shown in FIGS. 29A-29D, there was no significant change of bodyweight, body temperature, complete blood counts, or cytokine levels serum chemistry prior to and post autologous infusion of GSI060 modified T cells, indicating good tolerance of GSI060 in the Cynomolgus monkey model.

Other CAR-T cells, such as those transduced with GS1052-GSI059 can be evaluated with the same cynomolgus monkey model.

### Example 13. Single-Domain Anti-PD-1 or Anti-CTLA-4 Antibody Expressed by Engineered Primary Human T Cells Augments CART Cytotoxicity Against Tumor Cells In Vitro

A lentiviral vector (“pLIC-1042”) encoding both an anti-PD-1 single-domain antibody (sdAb) named LPD-1-16 and the anti-EGFRvIII CAR (GSI026, described in Example 3) was designed. pLIC-1042 was produced using a lentiviral packaging system in 293T cells as described in the examples above. Human primary T cells were prepared from PBMC using a Pan T cell isolation kit (Miltenyi, Cat#130-096-535). The isolated T cells were pre-activated with a T cell activation and expansion kit for 3 days. The following experimental groups of T cells were prepared: (1) T/pLIC-1042: engineered human primary T cells transduced with the lentiviral vector carrying both an anti-EGFRvIII-CAR gene (GSI026) and an anti-PD-1 sdAb (LPD-1-16) gene under the control of an NFAT promoter; (2) T/GSI026·anti-PD-1: engineered human primary T cells transduced with a lentiviral vector carrying both an anti-EGFRvIII-CAR gene (GSI026) and an IgG anti-PD-1 antibody (pembrolizumab) gene under the control of an NFAT promoter (as described in Example 3); (3) T/GSI026: engineered human primary T cells transduced with a lentiviral vector carrying an anti-EGFRvIII-CAR gene (GSI026); and (4) UnT: engineered human primary T cells transduced with an irrelevant gene as a negative control.

Each group of engineered primary human T cells (“effector cells”) was co-cultured with U87MG/VIII-Luc-PD-L1 cells (“target cells”) at an E/T ratio of 20:1 for 3 days. Cytotoxicity of the antibody-secreting primary human T cells on tumor cells was monitored by determining the remaining luciferase activity using the ONE-GLO™ luminescent assay kit according to the manufacturer's protocol. A low RLU value in the assay indicates strong cytotoxic efficacy of engineered T cells against target cells.

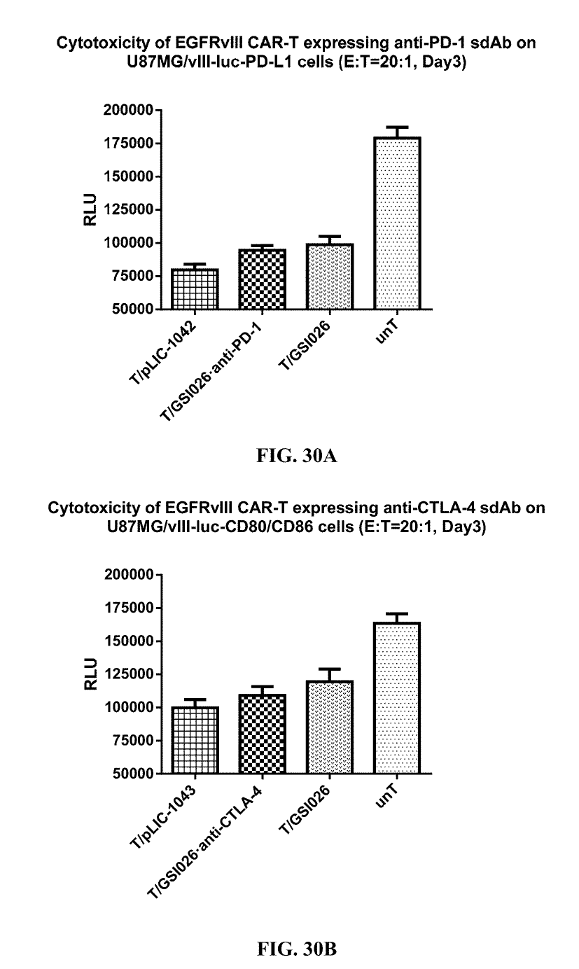

As shown in FIG. 30A, when co-cultured with target cells for 3 days, cells co-expressing both anti-EGFRvIII-CAR and anti-PD-1 sdAb (T/pLIC-1042) showed more potent cytotoxicity (RLU=79841±3646) against U87MG/VIII-Luc-PD-L1 tumor cells than cells expressing anti-EGFRvIII-CAR alone (T/GSI026, RLU=98874±6193) or T cells expressing IgG anti-PD-1 antibody and anti-EGFRvIII-CAR (T/GSI026 anti-PD-1, RLU=94599±3507).

Similarly, a lentiviral vector (“pLIC-1043”) encoding both an anti-CTLA-4 single-domain antibody (sdAb) named LCA-16 and the anti-EGFRvIII CAR (GSI026, described in Example 3) was designed. pLIC-1043 was produced using a lentiviral packaging system in 293T cells as described in the examples above. Human primary T cells were prepared from PBMC using a Pan T cell isolation kit (Miltenyi, Cat#130-096-535). The isolated T cells were pre-activated with a T cell activation and expansion kit for 3 days. The following experimental groups of T cells were prepared: (1) T/pLIC-1043: engineered human primary T cells transduced with the lentiviral vector carrying both an anti-EGFRvIII-CAR gene (GSI026) and an anti-CTLA-4 sdAb (LCA-16) gene under the control of an NFAT promoter; (2) T/GSI026·CTLA-4: engineered human primary T cells transduced with a lentiviral vector carrying both an anti-EGFRvIII-CAR gene (GSI026) and an IgG anti-CTLA-4 antibody (Ipilimumab) gene under the control of an NFAT promoter (as described in Example 4); (3) T/GSI026: engineered human primary T cells transduced with a lentiviral vector carrying an anti-EGFRvIII-CAR gene (GSI026); and (4) UnT: engineered human primary T cells transduced with an irrelevant gene as a negative control.

Each group of engineered primary human T cells (“effector cells”) was co-cultured with U87MG/VIII-Luc-CD80/CD86 cells (“target cells”) at an E/T ratio of 20:1 for 3 days. Cytotoxicity of the antibody-secreting primary human T cells on tumor cells was monitored by determining the remaining luciferase activity using the ONE-GLO™ luminescent assay kit according to the manufacturer's protocol. A low RLU value in the assay indicates strong cytotoxic efficacy of engineered T cells against target cells.

As shown in FIG. 30B, when co-cultured with U87MG/VIII-Luc-CD80/CD86 cells for 3 days, CART cells transduced with h pLIC-1043 vector expressing both anti-EGFRvIII-CAR and anti-CTLA-4 sdAb (T/pLIC-1043) showed more potent cytotoxicity (RLU=99816±6295) against tumor cells than CAR-T cells expressing anti-EGFRvIII-CAR alone (T/GSI026, RLU=119378±9670) or T cells expressing IgG anti-CTLA-4 antibody and anti-EGFRvIII-CAR (T/GSI026-anti-CTLA-4, RLU=109135±6695).

These data indicate that compared to full IgG anti-PD-1 (or anti-CTLA-4) antibody, single-domain anti-PD-1 (or anti-CTLA-4) antibody expressed by engineered CAR-T cells had more pronounced enhancement effect on the cytotoxicity of the CAR-T cells against tumor cells in vitro.

1. A pharmaceutical composition comprising: a) an engineered mammalian cell comprising a heterologous nucleic acid encoding an immunomodulator, wherein the heterologous nucleic acid is operably linked to a promoter; and b) a pharmaceutically acceptable excipient.

2. The pharmaceutical composition of claim 1, wherein the heterologous nucleic acid is present in the genome of the engineered mammalian cell.

3-4. (canceled)

5. The pharmaceutical composition of claim 1, wherein the engineered mammalian cell is an immune cell, a stem cell, or a primary cell.

6. The pharmaceutical composition of claim 5, wherein the immune cell is a peripheral blood monocyte cell (PBMC), T cell, B cell, or NK cell.

7. (canceled)

8. The pharmaceutical composition of claim 5, wherein the engineered mammalian cell further expresses a chimeric antigen receptor (CAR) or a recombinant T cell receptor (TCR).

9. The pharmaceutical composition of claim 8, wherein the engineered mammalian cell comprises a vector comprising the heterologous nucleic acid encoding the immunomodulator and a second heterologous nucleic acid encoding the CAR or the TCR, wherein the second heterologous nucleic acid encoding the CAR or the TCR is operably linked to the promoter.

10. (canceled)

11. The pharmaceutical composition of claim 9, wherein the promoter is inducible by an intracellular signaling domain of the CAR or the TCR.

12. The pharmaceutical composition of claim 8, wherein the CAR or TCR comprises an intracellular signaling domain with an abolished or attenuated immune effector function.

13. The pharmaceutical composition of claim 1, wherein the pharmaceutical composition further comprises a second cell, wherein the second cell is a mammalian immune cell expressing a chimeric antigen receptor (CAR) or a recombinant T cell receptor (TCR).

14-15. (canceled)

16. The pharmaceutical composition of claim 1, wherein the promoter is selected from an endogenous promoter, a heterologous promoter, or a promoter inducible by an inducing condition.

17. The pharmaceutical composition of claim 16, wherein the inducing condition is selected from the group consisting of: inducer, irradiation, temperature, redox state, tumor environment, and the activation state of the engineered mammalian cell.

18. (canceled)

19. The pharmaceutical composition of claim 16, wherein the promoter is a T cell activation-dependent promoter.

20-23. (canceled)

24. The pharmaceutical composition of claim 1, wherein the engineered mammalian cell further expresses on its surface a targeting molecule recognizing a tumor antigen.

25. The pharmaceutical composition of claim 1, wherein the immunomodulator is selected from an immune checkpoint inhibitor, an immunoactivator, or an antibody.

26. The pharmaceutical composition of claim 25, wherein the immune checkpoint inhibitor is an inhibitor of PD-1, PD-L1, PD-L2, CTLA-4, BLTA, TIM-3, or LAG-3.

27. (canceled)

28. The method of claim 25, wherein the immunoactivator is selected from the group consisting of IL-2, IL-7, IL-15, IL-21, IL-12, CCR4, CCR2b, Heparanase, CD137L, LEM, and Bcl-2.

29. (canceled)

30. The pharmaceutical composition of claim 25, wherein the antibody is a single chain antibody or a single-domain antibody.

31. (canceled)

32. The pharmaceutical composition of claim 25, wherein the antibody comprises a heavy chain and a light chain.

33. The pharmaceutical composition of claim 32, wherein the nucleic acid encoding the heavy chain and the nucleic acid encoding the light chain are operably linked to the same promoter or different promoters.

34. (canceled)

35. The pharmaceutical composition of claim 33, wherein the promoter for the nucleic acid encoding the heavy chain and the promoter for the nucleic acid encoding the light chain can be simultaneously or sequentially induced.

36. (canceled)

37. The pharmaceutical composition of claim 33, wherein the promoter for the nucleic acid encoding the heavy chain and the promoter for the nucleic acid encoding the light chain have a strength ratio of about 10:1 to about 1:10.

38. The pharmaceutical composition of claim 1, wherein the engineered mammalian cell further comprises a second heterologous nucleic acid encoding at least one therapeutic protein.

39. The pharmaceutical composition of claim 38, wherein the heterologous nucleic acid encoding the immunomodulator and the second heterologous nucleic acid encoding the therapeutic protein are operably linked to the same promoter or to different promoters.

40-43. (canceled)

44. A method of treating a cancer in an individual, comprising administering to the individual an effective amount of the pharmaceutical composition of claim 1.

45. The method of claim 44, wherein the pharmaceutical composition is administered systemically or locally to a site of a tumor.

46. The method of claim 45, wherein the systemically administered pharmaceutical composition is administered by infusion and the locally administered pharmaceutical composition is administered by injection.

47-48. (canceled)

49. The method of claim 44, further comprising inducing the expression of the immunomodulator in the engineered mammalian cell.

50. The method of claim 44, wherein the cancer is a solid tumor or a liquid tumor.

51. (canceled)

52. The method of claim 44, wherein the engineered mammalian cell is obtained from the individual.

53-54. (canceled)

55. A method of preparing the pharmaceutical composition of claim 1, comprising introducing into a mammalian cell a vector comprising the heterologous nucleic acid encoding the immunomodulator.

56. The method of claim 55, wherein the vector is a viral vector.

57-60. (canceled)
